SEQUENCE LISTING (139 sequences)

SEQ ID NO: 1 - protein, 211 aa, Homo sapiens
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLP

SEQ ID NO: 2 - protein, 236 aa, Homo sapiens
SEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLP

SEQ ID NO: 3 - protein, 20 aa, Homo sapiens
MEPWPLLLLFSLCSAGLVLG

SEQ ID NO: 4 - protein, 222 aa, Homo sapiens
SEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWEIIHKPSRLIQPPGDPRGGREGQRQEVIFYLIIRRKP

SEQ ID NO: 5 - protein, 23 aa, Homo sapiens
MTPGALLMLLGALGAPLAPGVRG

SEQ ID NO: 6 - protein, 219 aa, Homo sapiens
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKP

SEQ ID NO: 7 - protein, 22 aa, Homo sapiens
MHGGQGPLLLLLLLAVCLGAQG

SEQ ID NO: 8 - protein, 225 aa, Homo sapiens
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSCNVLVYHYGFVYWLPPAIFRSSCPISVTYFPFDWQNCSLKFSSLKYTAKEITLSLKQDAKENRTYPVEWIIIDPEGFTENGEWEIVHRPARVNVDPRAPLDSPSRQDITFYLIIRRKP

SEQ ID NO: 9 - protein, 21 aa, Homo sapiens
MEGPVLTLGLLAALAVCGSWG

SEQ ID NO: 10 - protein, 220 aa, Homo sapiens
KNEELRLYHHLFNNYDPGSRPVREPEDTVTISLKVTLTNLISLNEKEETLTTSVWIGIDWQDYRLNYSKDDFGGIETLRVPSELVWLPEIVLENNIDGQFGVAYDANVLVYEGGSVTWLPPAIYRSVCAVEVTYFPFDWQNCSLIFRSQTYNAEEVEFTFAVDNDGKTINKIDIDTEAYTENGEWAIDFCPGVIRRHHGGATDGPGETDVIYSLIIRRKP

SEQ ID NO: 11 - protein, 20 aa, Homo sapiens
MARAPLGVLLLLGLLGRGVG

SEQ ID NO: 12 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 120 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 14 - protein, 19 aa, Homo sapiens
MEFGLSWLFLVAILKGVQC

SEQ ID NO: 15 - protein, 19 aa, Mus musculus
MGWSCIILFLVATATGVHS

SEQ ID NO: 16 - protein, 22 aa, Homo sapiens
EPKSCDKTHTCPPCPAPELLGG

SEQ ID NO: 17 - protein, 18 aa, Homo sapiens
EPKCCVECPPCPAPPAAA

SEQ ID NO: 18 - protein, 19 aa, Homo sapiens
ESKYGPPCPPCPAPEFLGG

SEQ ID NO: 19 - protein, 70 aa, Homo sapiens
ELKTPLGDTTHTCPRCPEPKSCDTPPPCPRCPEPKSCDTPPPCPRCPEPKSCDTPPPCPRCPAPELLGGP

SEQ ID NO: 20 - protein, 5 aa, synthetic construct
  REGION 1..5: note = Synthetic
  VAR_SEQ 3: note = X - X is selected from P and R
CPXCP

SEQ ID NO: 21 - protein, 5 aa, Homo sapiens
CPPCP

SEQ ID NO: 22 - protein, 5 aa, Homo sapiens
CPRCP

SEQ ID NO: 23 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 24 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 25 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 26 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 27 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 29 - protein, 102 aa, Homo sapiens
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 30 - protein, 102 aa, Homo sapiens
SVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 31 - protein, 102 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 32 - protein, 102 aa, Homo sapiens
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 33 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 35 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 37 - protein, 15 aa, Homo sapiens
EPKSCDKTHTCPPCP

SEQ ID NO: 38 - protein, 5 aa, Homo sapiens
GGGGS

SEQ ID NO: 39 - protein, 15 aa, Homo sapiens
CEIIVTHFPFDEQNC

SEQ ID NO: 40 - protein, 15 aa, Homo sapiens
CSIQVTYFPFDWQNC

SEQ ID NO: 41 - protein, 15 aa, Homo sapiens
CSISVTYFPFDWQNC

SEQ ID NO: 42 - protein, 15 aa, Homo sapiens
CPISVTYFPFDWQNC

SEQ ID NO: 43 - protein, 15 aa, Homo sapiens
CAVEVTYFPFDWQNC

SEQ ID NO: 44 - protein, 15 aa, synthetic construct
  REGION 1..15: note = Synthetic
CDVSGVDTESGATNC

SEQ ID NO: 45 - protein, 12 aa, Homo sapiens
GSAGSAAGSGEF

SEQ ID NO: 46 - protein, 107 aa, Homo sapiens
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 107 aa, Homo sapiens
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 107 aa, Homo sapiens
GQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 107 aa, Homo sapiens
GQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 50 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 55 - protein, 331 aa, synthetic construct
  REGION 1..331: note = Synthetic
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 333 aa, synthetic construct
  REGION 1..333: note = Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 331 aa, synthetic construct
  REGION 1..331: note = Synthetic
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 331 aa, synthetic construct
  REGION 1..331: note = Synthetic
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 333 aa, synthetic construct
  REGION 1..333: note = Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 333 aa, synthetic construct
  REGION 1..333: note = Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 27 aa, Homo sapiens
MFMCLEGGEKNLTVLVSSAVSAGLVLG

SEQ ID NO: 62 - protein, 107 aa, Homo sapiens; identical to SEQ ID NO: 33

SEQ ID NO: 63 - protein, 15 aa, Homo sapiens
EPKSSDKTHTCPPCP

SEQ ID NO: 64 - protein, 330 aa, synthetic construct
  REGION 1..330: note = Synthetic
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 567 aa, synthetic construct
  REGION 1..567: note = Synthetic
SEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCDVSGVDTESGATNCTMVFSSYSYDSSEVSLQTGLGPDGQGHQEIHIHEGTFIENGQWEIIHKPSRLIQPPGDPRGGREGQRQEVIFYLIIRRKPGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 567 aa, synthetic construct
  REGION 1..567: note = Synthetic
SEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCDVSGVDTESGATNCTMVFSSYSYDSSEVSLQTGLGPDGQGHQEIHIHEGTFIENGQWEIIHKPSRLIQPPGDPRGGREGQRQEVIFYLIIRRKPGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 564 aa, synthetic construct
  REGION 1..564: note = Synthetic
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - protein, 565 aa, synthetic construct
  REGION 1..565: note = Synthetic
KNEELRLYHHLFNNYDPGSRPVREPEDTVTISLKVTLTNLISLNEKEETLTTSVWIGIDWQDYRLNYSKDDFGGIETLRVPSELVWLPEIVLENNIDGQFGVAYDANVLVYEGGSVTWLPPAIYRSVCDVSGVDTESGATNCSLIFRSQTYNAEEVEFTFAVDNDGKTINKIDIDTEAYTENGEWAIDFCPGVIRRHHGGATDGPGETDVIYSLIIRRKPGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 564 aa, synthetic construct
  REGION 1..564: note = Synthetic
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 19 aa, Homo sapiens
MEWSWVFLFFLSVTTGVHS

SEQ ID NO: 71 - protein, 20 aa, Homo sapiens
MSVPTQVLGLLLLWLTDARC

SEQ ID NO: 72 - protein, 210 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATCSMKLGTWTYDGSVVAINPESDQPDLSNFMESGEWVIKESRGWKHSVTYSCCPDTPYLDITYHFVMQRLP

SEQ ID NO: 73 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDGAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLP

SEQ ID NO: 74 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLP

SEQ ID NO: 75 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSAVAINPESDQPDLSNFMESGEWVIKESRGWKHSVTYSCCPDTPYLDITYHFVMQRLP

SEQ ID NO: 76 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLFNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLP

SEQ ID NO: 77 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLHNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLP

SEQ ID NO: 78 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLRNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLP

SEQ ID NO: 79 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALYCNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 80 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 81 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 82 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 83 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIEWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 84 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 85 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIEWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 86 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 87 - protein, 225 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKP

SEQ ID NO: 88 - protein, 225 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSINVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKP

SEQ ID NO: 89 - protein, 225 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSCNVLVYHYGFVRWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKP

SEQ ID NO: 90 - protein, 224 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVRWLPPAIFRSSCDVSGVDTESGATCSLKFSSLKYTAKEITLSLKQDAKENRTYPVEWIIIDPEGFTENGEWEIVHRPARVNVDPRAPLDSPSRQDITFYLIIRRKP

SEQ ID NO: 91 - protein, 225 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVRWLPPAIFRSSCDVSGVDTESGATNCSLKFSSEKYTAKEITLSLKQDAKENRTYPVEWIIIDPEGFTENGEWEIVHRPARVNVDPRAPLDSPSRQDITFYLIIRRKP

SEQ ID NO: 92 - protein, 458 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 466 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 713 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 706 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 471 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 711 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 466 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 458 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 472 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 472 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 106 - protein, 713 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVRWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 713 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSEKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 962 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSGGGGSGGGGSGGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 956 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSGGGGSGGGGSGGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLHNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLHNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 704 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSESKYGPPCPPCPAPEFLGGPCVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 956 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSGGGGSGGGGSGGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 956 aa, synthetic construct
RNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSGGGGSGGGGSGGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 692 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 701 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGSGGGSGGGSGGGSGGGSGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 703 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGSGGGSGGGSGGGSGGGSGGGSGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 699 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGSGGGSGGGSGGGSGGGSGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 690 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPEAAAKEAAAKGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 687 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPEAAAKEAAAKGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGSGGGSGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 687 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPEAAAKEAAAKGGSRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGSGGGSGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 699 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSGGGGSGGGGSGGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLP

SEQ ID NO: 125 - protein, 699 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 126 - protein, 689 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGSGGGSGGGSGGGSGGGSSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTRTYDGSVVAINPESDQPDLSNFMESGEWVIKESRGWKHSVTYSCCPDTPYLDITYHFVMQRLP

SEQ ID NO: 127 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 707 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSRNQEERLLADLMQNYDPNLRPAERDSDVVNVSLKLTLTNLISLNEREEALTTNVWIEMQWCDYRLRWDPRDYEGLWVLRVPSTSVWRPDIVLENNVDGVFEVALECNVLVSPDGCIRWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKPGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 713 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLPGGGGSGGGGSGGGGSGGGGSGGGGSGGGGSLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSANVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKPGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 211 aa, synthetic construct
SEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCDVSGVDTESGATNCSMKLGTWTYDGSVVAINPESDQPDLSNFMESGEWVIKESRGWKHSVTYSCCPDTPYLDITYHFVMQRLP

SEQ ID NO: 132 - protein, 222 aa, synthetic construct
SEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCDVSGVDTESGATNCTMVFSSYSYDSSEVSLQTGLGPDGQGHQEIHIHEGTFIENGQWEIIHKPSRLIQPPGDPRGGREGQRQEVIFYLIIRRKP

SEQ ID NO: 133 - protein, 219 aa, synthetic construct
RNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACDVSGVDTESGATNCSLIFQSQTYSTNEIDLQLSQEDGQTIEWIFIDPEAFTENGEWAIQHRPAKMLLDPAAPAQEAGHQKVVFYLLIQRKP

SEQ ID NO: 134 - protein, 225 aa, synthetic construct
LNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSCNVLVYHYGFVYWLPPAIFRSSCDVSGVDTESGATNCSLKFSSLKYTAKEITLSLKQDAKENRTYPVEWIIIDPEGFTENGEWEIVHRPARVNVDPRAPLDSPSRQDITFYLIIRRKP

SEQ ID NO: 135 - protein, 220 aa, synthetic construct
KNEELRLYHHLFNNYDPGSRPVREPEDTVTISLKVTLTNLISLNEKEETLTTSVWIGIDWQDYRLNYSKDDFGGIETLRVPSELVWLPEIVLENNIDGQFGVAYDANVLVYEGGSVTWLPPAIYRSVCDVSGVDTESGATNCSLIFRSQTYNAEEVEFTFAVDNDGKTINKIDIDTEAYTENGEWAIDFCPGVIRRHHGGATDGPGETDVIYSLIIRRKP

SEQ ID NO: 136 - protein, 5 aa, synthetic construct
GGGGS

SEQ ID NO: 137 - protein, 5 aa, synthetic construct
EAAAK

SEQ ID NO: 138 - protein, 26 aa, synthetic construct
HHHHHHHHPGSGLNDIFEAQKIEWHE

SEQ ID NO: 139 - protein, 5 aa, synthetic construct
LPETG

(19) United States
(12) Patent Application Publication
(10) Pub. No.: US 2025/0059255 A1
(43) Pub. Date: Feb. 20, 2025

(54) IG-LIKE FUSION PROTEINS AND USE THEREOF

(71) Applicant: CANOPY IMMUNO-THERAPEUTICS LTD., Megadim (IL)

(72) Inventors: Kfir OVED, Megadim (IL); Galit DENKBERG, Nofit (IL); Sharon REEF, Tzukei Yam (IL); Yelena PINZUR, Haifa (IL)

(21) Appl. No.: 18/719,727

(22) PCT Filed: Dec. 13, 2022
(86) PCT No.: PCT/IL2022/051321

§ 371 Date: Jun. 13, 2024

Related U.S. Application Data
(60) Provisional application No. 63/288,782, filed on Dec. 13, 2021.

(51) Int. Cl. C07K 14/705; A61K 38/00; A61P 21/04; A61P 37/06

(52) U.S. Cl. CPC C07K 14/70571; A61P 21/04; A61P 37/06; A61K 38/00; C07K 2319/30

(57) ABSTRACT

Compositions comprising a fragment of a first human receptor target of myasthenia gravis autoantibodies or an analog or derivative thereof and a fragment of a second human protein receptor of myasthenia gravis autoantibodies or an analog or derivative thereof are provided. Pharmaceutical compositions comprising the composition, nucleic acid systems encoding the polypeptides of the composition and methods of treatment and determining suitability for treatment using the composition; as well as methods of producing the composition are also provided.

### CROSS REFERENCE TO RELATED APPLICATIONS

This application claims the benefit of priority of U.S. Provisional Patent Application No. 63/288,782, filed Dec. 13, 2021, the contents of which are incorporated herein by reference in its entirety.

### REFERENCE TO AN ELECTRONIC SEQUENCE LISTING

The contents of the electronic sequence listing (CNPY-P-002-PCT.xml; Size: 170,370 bytes; and Date of Creation: Dec. 12, 2022) is herein incorporated by reference in its entirety.

### FIELD OF INVENTION

The present invention is in the field of fusion protein generation and myasthenia gravis treatment.

### BACKGROUND OF THE INVENTION

Myasthenia gravis (MG) is the most common neuromuscular transmission disorder. The age of onset is bimodal with a first peak in the second and third decades (female predominance) and a second peak in the sixth to eighth decade (male predominance). MG is an autoimmune disease characterized by weakness of skeletal muscles due to disruption of neuromuscular junction function. ˜85% of MG patients have acetylcholine receptor (AChR) specific antibodies in their serum, which act as AChR antagonists, cause receptor clustering and internalization, and recruit complement which subsequently cause tissue damage. Individual patients have a mix of different antibodies to the AChR. Some patients with AChR antibody-positive MG also have thymic abnormalities with roughly two thirds with hyperplasia, and 10% with thymoma. The predominant clinical feature of MG is fluctuating skeletal muscle weakness, often with true muscle fatigue.

There are two clinical forms of myasthenia gravis: ocular and generalized. In ocular myasthenia, the weakness is limited to the eyelids and extraocular muscles. In generalized disease, the weakness may also affect ocular muscles, but it also involves a variable combination of bulbar, limb, and respiratory muscles. Transient worsening of symptoms can be triggered by infection, surgery, pregnancy, childbirth, medications, tapering of immunosuppressive medications, or as part of the natural progression of the disease. Respiratory muscles involvement is the most serious symptom of myasthenia gravis, that might lead to a respiratory insufficiency and pending respiratory failure, called “myasthenic crisis”. There is also a long list of drugs that must be avoided in MG patients including fluoroquinolone, aminoglycoside, magnesium sulfate, hydroxychloroquine, penicillamine, and botulinum toxin. Beta blockers, procainamide, quinidine, and quinine should also be avoided when possible. The therapies for MG include acetylcholinesterase inhibitor (pyridostigmine), chronic immunosuppressive therapies, rapid and transient immunomodulatory therapies (e.g. plasma exchange and intravenous immune globulin-IVIG), and thymectomy. The treatment goal is to allow the patient minimal symptoms with minimal drug related side effects; however, no cure is available. Initial symptomatic therapy in patients with MG is based on acetylcholinesterase inhibitor (e.g., pyridostigmine). Cholinergic adverse effects of pyridostigmine can be dose-limiting in many patients and comprise abdominal cramping and diarrhea. Most patients with generalized MG require additional therapy with glucocorticoids and/or other immunosuppressive drugs, though this is a second line therapy. Therapeutic plasma exchange (plasmapheresis) and IVIG have a prompt effect but a short duration. New methods of treating MG, especially ones that have a long-lasting effect, are greatly needed. Specifically, therapies that target the autoantibodies that cause MG are needed, and beyond this, drugs that can directly target the autoreactive B cells/plasma cells that are the source of these autoantibodies and potentially cure the conditions are greatly needed.

### SUMMARY OF THE INVENTION

The present invention provides compositions comprising a fragment of a first human acetylcholine receptor subunit and a fragment of a second human acetylcholine receptor subunit.

According to a first aspect, there is provided a composition, comprising a fragment of a first human acetylcholine receptor subunit or an analog or derivative thereof, and a fragment of a second human acetylcholine receptor subunit or an analog or derivative thereof, and wherein the first and second subunits are different subunits.

According to another aspect, there is provided a composition, comprising a fragment of a first human acetylcholine receptor subunit or an analog or derivative thereof, and a fragment of a second human acetylcholine receptor subunit or an analog or derivative thereof, and wherein the first and second subunits are different subunits.

According to another aspect, there is provided a polypeptide comprising a fragment of a first human acetylcholine receptor subunit comprising at least one mutation that decreases aggregation of the fragment, wherein said fragment is selected from:
a. a fragment of ACHRA and comprises a mutation selected from: deletion of N141, F100G, W149R, V155A, Y93F, Y93H, Y93R and a combination thereof within a wild-type AChRa comprising SEQ ID NO: 1 or a AChRa with increased solubility comprising SEQ ID NO: 131;
b. a fragment of ACHRG and comprises a mutation selected from: M84S, Y105E, Y117E, Y117R, and a combination thereof within a wild-type AChRa comprising SEQ ID NO: 6 or a AChRa with increased solubility comprising SEQ ID NO: 133; and
c. a fragment of ACHRD and comprises a mutation selected from: C108A, C108I, Y119R, deletion of N141, L151E and a combination thereof within a wild-type AChRa comprising SEQ ID NO: 8 or a AChRa with increased solubility comprising SEQ ID NO: 134.

According to some embodiments, the first human protein, the second human protein or both is a cell surface receptor, and the fragment is a fragment of an extracellular domain of the receptor.

According to some embodiments, the fragment is a fragment of an extracellular domain of the acetylcholine receptor subunit.

According to some embodiments, the first and second acetylcholine receptor subunits are selected from acetylcholine receptor subunit alpha (ACHRA), acetylcholine receptor subunit beta (ACHRB), acetylcholine receptor subunit gamma (ACHRG), acetylcholine receptor subunit delta (ACHRD) and acetylcholine receptor subunit epsilon (ACHRE).

According to some embodiments, the fragment of a first human protein and the fragment of a second human protein comprise different sequences.

According to some embodiments, the composition of the invention comprises a protein complex comprising:
a. a first polypeptide chain comprising the fragment of a first human acetylcholine receptor subunit or an analog or derivative thereof and a first dimerization domain; and
b. a second polypeptide chain comprising the fragment of a second human acetylcholine receptor subunit or an analog or derivative thereof and second dimerization domain;
wherein the first and second dimerization domains are configured to dimerize with each other.

According to some embodiments, the composition of the invention comprises a protein complex comprising:
a. a first polypeptide chain comprising the fragment of a first human acetylcholine receptor subunit or an analog or derivative thereof and a first dimerization domain; and
b. a second polypeptide chain comprising the fragment of a second human acetylcholine receptor subunit or an analog or derivative thereof and second dimerization domain;
wherein the first and second dimerization domains are configured to dimerize with each other.

According to some embodiments, the dimerizing comprises forming a covalent bond between the first dimerization domain and the second dimerization domain.

According to some embodiments, the protein complex comprises an immunoglobulin scaffold.

According to some embodiments,
a. the first dimerization domain comprises a first hinge domain of a heavy chain of an immunoglobulin and the second dimerization domain comprises a second hinge domain of a heavy chain and the first and the second dimerization domains dimerizes by a disulfide bond; or
b. the first and second dimerization domains each comprise a domain selected from a CH1 domain of a heavy chain of an immunoglobulin and a CL domain of a light chain of an immunoglobulin and dimerize by a disulfide bond and wherein the first and second dimerization domains do not both comprise the CH1 domain or the CL domain.

According to some embodiments, the fragment and the dimerization domain of the first, second or both polypeptide chains are separated by a linker.

According to some embodiments, the first polypeptide chain, the second polypeptide chain or both further comprise an Fc region of a human antibody heavy chain.

According to some embodiments, the Fc region is capable of inducing cytotoxicity against a cell binding the protein complex.

According to some embodiments, the first polypeptide chain comprises a first CH3 domain of a heavy chain of an immunoglobulin, a first CH2 domain of a heavy chain of an immunoglobulin or both and the second polypeptide chain comprises a second CH3 domain of a heavy chain of an immunoglobulin, a second CH2 domain of a heavy chain of an immunoglobulin or both.

According to some embodiments, the first CH3 domain comprises at least a first mutation and the second CH3 domain comprises at least a second mutation, and wherein the mutations permit heterodimerization of the first and second polypeptide chains and inhibit homodimerization of the first polypeptide chain and homodimerization of the second polypeptide chain.

According to some embodiments, the first CH2 domain comprises at least a first mutation and the second CH2 domain comprises at least a second mutation, and wherein the mutations permit heterodimerization of the first and second polypeptide chains and inhibit homodimerization of the first polypeptide chain and homodimerization of the second polypeptide chain.

According to some embodiments, the first mutation is selected from a mutation provided in Table 1 and the second mutation is provided in Table 1 and is a corresponding mutation to the first mutation.

According to some embodiments, the Fc is from an IgG2 or IgG4 or comprises at least one mutation that reduces effector function.

According to some embodiments, the Fc region of the first, second or both polypeptide chains is separated from the fragment or the dimerization domain by a linker.

According to some embodiments, the dimerization domain of the first, second or both polypeptide chains is C-terminal to the fragment and N-terminal to the Fc region. According to some embodiments, the dimerization domain of the first, second or both polypeptide chains is C-terminal to the fragment. According to some embodiments, the dimerization domain of the first, second or both polypeptide chains is N-terminal to the fragment.

According to some embodiments, the composition of the invention is devoid of an antibody variable domain.

According to some embodiments, the composition of the invention further comprises a third polypeptide comprising a fragment of a third human acetylcholine receptor subunit, or an analog or derivative thereof and a third dimerization domain, wherein the first polypeptide further comprises a fourth dimerization domain and the third and fourth dimerization domains are capable of dimerizing to each other.

According to some embodiments, the composition of the invention further comprises a third polypeptide comprising a fragment of a third human acetylcholine receptor subunit, or an analog or derivative thereof and a third dimerization domain, wherein the first polypeptide further comprises a fourth dimerization domain and the third and fourth dimerization domains are capable of dimerizing to each other.

According to some embodiments,
a. the third dimerization domain comprises a first hinge domain of a heavy chain of an immunoglobulin and the fourth dimerization domain comprises a second hinge domain of a heavy chain and the first and the second dimerization domains dimerizes by a disulfide bond; or
b. the third and fourth dimerization domains each comprise a domain selected from a CH1 domain of a heavy chain of an immunoglobulin and a CL domain of a light chain of an immunoglobulin and dimerize by a disulfide bond and wherein the first and third polypeptides do not both comprise the CH1 domain or the CL domain.

According to some embodiments, the composition of the invention further comprises a fourth polypeptide comprising a fragment of a fourth human acetylcholine receptor subunit, or an analog or derivative thereof and a fifth dimerization domain, wherein the second polypeptide further comprises a sixth dimerization domain and the fifth and six dimerization domains are capable of dimerizing to each other.

According to some embodiments, the composition of the invention further comprises a fourth polypeptide comprising a fragment of a fourth human acetylcholine receptor subunit, or an analog or derivative thereof and a fifth dimerization domain, wherein the second polypeptide further comprises a sixth dimerization domain and the fifth and six dimerization domains are capable of dimerizing to each other.

According to some embodiments,
a. the fifth dimerization domain comprises a first hinge domain of a heavy chain of an immunoglobulin and the sixth dimerization domain comprises a second hinge domain of a heavy chain and the first and the second dimerization domains dimerizes by a disulfide bond; or
b. the fifth and six dimerization domains each comprise a domain selected from a CH1 domain of a heavy chain of an immunoglobulin and a CL domain of a light chain of an immunoglobulin and dimerize by a disulfide bond and wherein the first and third polypeptides do not both comprise the CH1 domain or the CL domain.

According to some embodiments, the first polypeptide and the second polypeptide do not both comprise a CH1 domain or both comprise a CL domain.

According to some embodiments, the third and fourth dimerization domains or the fifth and sixth dimerization domains comprise mutations that permits dimerization of the third and fourth dimerization domains and the fifth and sixth dimerization domains and inhibit dimerization of the third dimerization domain to the fifth or sixth dimerization domain and the sixth dimerization domain to the third or fourth dimerization domain.

According to some embodiments, the composition of the invention comprises a single polypeptide chain comprising the fragment of a first human acetylcholine receptor subunit or an analog or derivative thereof and the fragment of a second human acetylcholine receptor subunit or an analog or derivative thereof.

According to some embodiments, the composition of the invention comprises a single polypeptide chain comprising the fragment of a first human acetylcholine receptor subunit or an analog or derivative thereof and the fragment of a second human acetylcholine receptor subunit or an analog or derivative thereof.

According to some embodiments, the single polypeptide chain further comprises a fragment of a third acetylcholine receptor subunit or an analog or derivative thereof and optionally a fragment of a fourth acetylcholine receptor subunit or an analog or derivative thereof.

According to some embodiments, the single polypeptide chain further comprises a fragment of a third human acetylcholine receptor subunit or an analog or derivative thereof and optionally a fragment of a fourth human acetylcholine receptor subunit or an analog or derivative thereof.

According to some embodiments, the fragments are separate by an amino acid linker.

According to some embodiments, the linker is a flexible liner. According to some embodiments, the flexible linker is a GS linker. According to some embodiments, the linker is a rigid linker.

According to some embodiments, the polypeptide chain further comprises an Fc region of a human antibody heavy chain.

According to some embodiments, a second polypeptide chain comprises a third human acetylcholine receptor subunit or an analog or derivative thereof and an Fc region of a human antibody heavy chain.

According to some embodiments, the second polypeptide chain further comprises a fourth human acetylcholine receptor subunit.

According to some embodiments, the composition of the invention further comprises a second polypeptide chain comprises a third human acetylcholine receptor subunit or an analog or derivative thereof, optionally wherein said second polypeptide chain further comprises a fourth human acetylcholine receptor subunit.

According to some embodiments, the autoantibodies are against an epitope that spans at least two of the fragments.

According to some embodiments, the fragments of the first, second, and optionally the third and fourth human proteins are selected from fragments of extracellular domains of human receptors selected from the group consisting of: acetylcholine receptor (AChR) alpha, AChR beta, AChR gamma, AChR delta, and AChR epsilon.

According to some embodiments, the complex comprises at least one amino acid sequence selected from SEQ ID NO: 64 to 69 or a derivative thereof comprising at least 80% identity thereto. In some embodiments, at least 80% is at least 85%.

According to some embodiments, the fragments of the first, second, third and fourth human proteins comprise different sequences.

According to some embodiments, the first, second, and optionally the third and fourth proteins are part of a single protein complex in humans.

According to some embodiments, at least one of the fragments comprise a mutation in a ligand binding domain and wherein the mutation reduces binding to the ligand.

According to some embodiments, at least one of the fragments comprise a mutation that increases stability or solubility of the fragment.

According to some embodiments, the mutation comprises replacement of a cys loop within an acetylcholine receptor subunit with CDVSGVDTESGATNC (SEQ ID NO: 44).

According to some embodiments, the acetylcholine receptor subunit is selected from: an alpha subunit comprising the amino acid sequence provided in SEQ ID NO: 131, a beta subunit comprising the amino acid sequence provided in SEQ ID NO: 132, a gamma subunit comprising the amino acid sequence provided in SEQ ID NO: 133, a delta subunit comprising the amino acid sequence provided in SEQ ID NO: 134, and an epsilon subunit comprising the amino acid sequence provided in SEQ ID NO: 135.

According to some embodiments, an analog or derivative thereof comprises at least 80% identity to the human protein. In some embodiments, at least 80% is at least 85%.

According to some embodiments, the fragment comprises at least 20 sequential amino acids from the protein.

According to some embodiments, the fragment comprises at least one B cell receptor (BCR)-specific epitope target of the autoantibodies.

According to some embodiments, the fragment comprises at least one mutation that decreases aggregation of the fragment.

According to some embodiments, the fragment is selected from:
a. a fragment of ACHRA and comprises a mutation selected from: deletion of N141, F100G, W149R, V155A, Y93F, Y93H, Y93R and a combination thereof within a wild-type AChRa comprising SEQ ID NO: 1 or a AChRa with increased solubility comprising SEQ ID NO: 131;
b. a fragment of ACHRG and comprises a mutation selected from: M84S, Y105E, Y117E, Y117R, and a combination thereof within a wild-type AChRa comprising SEQ ID NO: 6 or a AChRa with increased solubility comprising SEQ ID NO: 133; and
c. a fragment of ACHRD and comprises a mutation selected from: C108A, C108I, Y119R, deletion of N141, L151E and a combination thereof within a wild-type AChRa comprising SEQ ID NO: 8 or a AChRa with increased solubility comprising SEQ ID NO: 134.

According to some embodiments, the first polypeptide chain and the second polypeptide chain are selected from: SEQ ID NO: 92 and SEQ ID NO: 93; SEQ ID NO: 95 and SEQ ID NO: 96; SEQ ID NO: 97 and SEQ ID NO: 98, SEQ ID NO: 99 and SEQ ID NO: 100, SEQ ID NO: 92 and SEQ ID NO: 102; SEQ ID NO 103 and SEQ ID NO: 100; SEQ ID NO: 105 and SEQ ID NO: 130; SEQ ID NO: 105 and SEQ ID NO: 106; and SEQ ID NO: 105 and SEQ ID NO: 107.

According to some embodiments, the single polypeptide chain is selected from: SEQ ID NO: 94, SEQ ID NO: 104, and SEQ ID NO: 108-129.

According to some embodiments, the polypeptide further comprises replacement of a cys loop within an acetylcholine receptor subunit with CDVSGVDTESGATNC (SEQ ID NO: 44) and wherein the subunit is ACHRA and the cys loop consists of CEIIVTHFPFDEQNC (SEQ ID NO: 39), the subunit is ACHRG and the cys loop consists of CSISVTYFPFDWQNC (SEQ ID NO: 41), or the subunit is ACHRD and the cys loop consists of CPISVTYFPFDWQNC (SEQ ID NO: 42).

According to some embodiments, the polypeptide further comprises a second fragment of a second acetylcholine receptor subunit linked to the first fragment by an amino acid linker. According to some embodiments, the polypeptide further comprises a fragment from a third, fourth, or fifth acetylcholine receptor subunit.

According to some embodiments, the polypeptide further comprises an Fc region of a human antibody heavy chain. According to some embodiments, the Fc region is separated from said fragment by an amino acid linker.

According to some embodiments, the polypeptide comprises a sequence selected from SEQ ID NO: 72-91.

According to another aspect, there is provided a pharmaceutical composition comprising a composition of the invention and a pharmaceutically acceptable carrier, excipient or adjuvant.

According to another aspect, there is provided a pharmaceutical composition comprising a polypeptide of the invention and a pharmaceutically acceptable carrier, excipient or adjuvant.

According to another aspect, there is provided a method of treating myasthenia gravis in a subject in need thereof, the method comprising administering to the subject a composition of the invention or a pharmaceutical composition of the invention, thereby treating myasthenia gravis.

According to some embodiments, the method of the invention, further comprises reducing in the subject the levels of circulating antibodies against at least the first human acetylcholine receptor subunit prior to the administering.

According to some embodiments, the method of the invention further comprises reducing in the subject the levels of circulating antibodies against a human acetylcholine receptor subunit within a protein complex comprising the first human acetylcholine receptor subunit or the second human acetylcholine receptor subunit.

According to some embodiments, the treating comprises decreasing the concentration of circulating autoantibodies against the human acetylcholine receptor subunits.

According to some embodiments, the treating comprises killing B cells producing the autoantibodies.

According to some embodiments, the composition comprises the Fc region and the treating comprises killing B cells producing the autoantibodies.

According to some embodiments, the B cells are autoreactive B cells producing autoantibodies against a fragment of the composition or polypeptide.

According to another aspect, there is provided a nucleic acid system comprising a nucleic acid molecule, wherein a first nucleic acid molecule encodes the first polypeptide chain of a composition of the invention and a second nucleic acid molecule encodes the second polypeptide chain of a composition of the invention.

According to another aspect, there is provided a nucleic acid system comprising a nucleic acid molecule that encodes a polypeptide of the invention.

According to some embodiments, the nucleic acid system of the invention, further comprising, a third nucleic acid molecule encoding the third polypeptide chain of a composition of the invention, a fourth nucleic acid molecule encoding the fourth polypeptide chain of a composition of the invention, or both.

According to another aspect, there is provided a nucleic acid molecule encoding a single polypeptide chain of a composition of the invention comprising a fragment of a first human acetylcholine receptor subunit or an analog or derivative thereof and a fragment of a second human acetylcholine receptor subunit or an analog or derivative thereof.

According to another aspect, there is provided method of producing a composition of the invention, the method comprising expressing the nucleic acid system of the invention or the nucleic acid molecule of the invention in a cell, wherein the nucleic acid system is configured to produce the encoded polypeptide in the cell, thereby producing a composition of the invention.

According to another aspect, there is provided method of producing a polypeptide of the invention, the method comprising expressing the nucleic acid system of the invention in a cell, wherein the nucleic acid system is configured to produce the encoded polypeptide in the cell, thereby producing a composition of the invention.

According to another aspect, there is provided a method for producing a protein the method comprising:
obtaining a first fragment of an extracellular domain of a first human acetylcholine receptor subunit or an analog or derivative thereof, and a second fragment of an extracellular domain of a second human acetylcholine receptor subunit or analog or derivative thereof, wherein the first and second human acetylcholine receptor subunit are different proteins, and linking the first fragment to the second fragment to produce a single polypeptide chain; or culturing a host cell comprising one or more vectors comprising a nucleic acid sequence encoding a single polypeptide chain, wherein the single polypeptide chain is produced by:
   i. obtaining a first fragment of an extracellular domain of a first human acetylcholine receptor subunit or an analog or derivative thereof and a second fragment of an extracellular domain of a second human acetylcholine receptor subunit or analog or derivative thereof, wherein the first and second human acetylcholine receptor subunit are different subunits; and
   ii. linking the first fragment to the second fragment to produce a single polypeptide chain;
thereby producing a protein.

According to another aspect, there is provided a method for producing a protein complex the method comprising:
obtaining a first fragment of an extracellular domain of a first human acetylcholine receptor subunit or an analog or derivative thereof, and a second fragment of an extracellular domain of a second human acetylcholine receptor subunit or analog or derivative thereof, wherein the first and second human acetylcholine receptor subunits are different proteins, linking the first fragment to a first dimerization domain to produce a first polypeptide chain and linking the second fragment to a second dimerization domain to produce a second polypeptide chain wherein the first and second dimerization domains are capable of dimerizing with each other and contacting the first polypeptide and the second polypeptide under conditions sufficient to induce the dimerization; or
culturing a host cell comprising one or more vectors comprising a nucleic acid sequence encoding at least two polypeptide chains, wherein the two polypeptide chains are produced by:
   i. obtaining a first fragment of an extracellular domain of a first human acetylcholine receptor subunit or an analog or derivative thereof and a second fragment of an extracellular domain of a second human acetylcholine receptor subunit or analog or derivative thereof, wherein the first and second human acetylcholine receptor subunit are different subunits; and
   ii. linking the first fragment to a first dimerization domain to produce a first polypeptide chain and linking the second fragment to a second dimerization domain to produce a second polypeptide chain wherein the first and second dimerization domains are capable of dimerizing with each other;
thereby producing a protein complex.

According to some embodiments, the protein is a single polypeptide chain of a composition of the invention.

According to some embodiments, the protein complex is a protein complex of a composition of the invention.

According to some embodiments, the method further comprises
a. linking a third dimerization domain to the first dimerization domain or first fragment within the first polypeptide chain; obtaining a third fragment of an extracellular domain of a third human acetylcholine receptor subunit or an analog or derivative thereof, and linking the third fragment to a fourth dimerization domain to produce a third polypeptide chain wherein the third dimerization domain and the fourth dimerization domain are capable of dimerizing to each other; and contacting the first, second, and third polypeptides under conditions sufficient to induce the dimerization; or
b. expressing in the host cell a nucleic acid sequence encoding a third polypeptide chain produced by:
   i. obtaining a third fragment of an extracellular domain of a third human acetylcholine receptor subunit or an analog or derivative thereof; and
   ii. linking the third fragment to a fourth dimerization domain to produce a third polypeptide chain;
wherein the first polypeptide chain further comprises a third dimerization domain and wherein the third dimerization domain and the fourth dimerization domain or capable of dimerizing to each other.

According to some embodiments, the method further comprises
a. linking a sixth dimerization domain to the second dimerization domain or second fragment within the second polypeptide chain; obtaining a fourth fragment of an extracellular domain of a fourth human acetylcholine receptor subunit or an analog or derivative thereof, and linking the fourth fragment to a fifth dimerization domain to produce a fourth polypeptide chain wherein the fifth dimerization domain and the sixth dimerization domain are capable of dimerizing to each other; and contacting the first, second, third and fourth polypeptides under conditions sufficient to induce the dimerization; or
b. expressing in the host cell a nucleic acid sequence encoding a fourth polypeptide chain produced by:
   i. obtaining a fourth fragment of an extracellular domain of a fourth human acetylcholine receptor subunit or an analog or derivative thereof; and
   ii. linking the fourth fragment to a fifth dimerization domain to produce a fourth polypeptide chain;
wherein the second polypeptide chain further comprises a sixth dimerization domain and wherein the fifth dimerization domain and the sixth dimerization domain or capable of dimerizing to each other.

According to another aspect, there is provided a method of producing a polypeptide, the method comprising:
a. obtaining a first fragment of an extracellular domain of a first human acetylcholine receptor subunit or an analog or derivative thereof; and
b. generating in the first fragment at least one mutation that decreases aggregation of the first fragment to produce a mutated first fragment;
thereby producing a polypeptide.

According to some embodiments, the method further comprises confirming decreased aggregation of the mutated first fragment.

According to some embodiments, the method further comprises confirming the at least one mutation does not substantially decrease binding of the first fragment to autoantibodies against the first fragment.

According to some embodiments, the method further comprises linking the mutated first fragment to an Fc region of a human antibody heavy chain, to a second fragment of an extracellular domain of a second human acetylcholine receptor subunit or an analog or derivative thereof, or both. According to some embodiments, the second fragment comprises at least one mutation that decreases aggregation of the second fragment.

According to some embodiments, an analog or derivative thereof comprises at least 80% identity to the human protein.

According to another aspect, there is provided a protein complex or protein produced by a method of the invention.

According to another aspect, there is provided a method of determining suitability of a subject in need thereof to be treated by a method of the invention, the method comprising receiving a sample from the subject, contacting the sample with a composition of the invention and determining binding of autoantibodies against AChR within the sample to the composition, wherein binding of autoantibodies against AChR to the composition indicates the subject is suitable to be treated by a method of the invention, thereby determining suitability of the subject to be treated.

According to another aspect, there is provided a method of determining suitability of a subject in need thereof to be treated by a method of the invention, the method comprising receiving a sample from the subject, contacting the sample with a polypeptide of the invention and determining binding of autoantibodies against AChR within the sample to the polypeptide, wherein binding of autoantibodies against AChR to the polypeptide indicates the subject is suitable to be treated by a method of the invention, thereby determining suitability of the subject to be treated.

According to some embodiments, binding of at least 20% of autoantibodies against AChR in the sample to a composition of the invention or a polypeptide of the invention indicates the subject is suitable to be treated by a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-G: (1A) A bar graph of the % depletion results as measured in an anti-AChR serology ELISA assay with and without serum depletion with Alpha1 ECD. The graph shows the results of 335 randomly selected MG patients where the y-axis shows the % depletion (defined as [100%−{anti-AChR cone (with prior alpha-ECD depletion)/anti-AChR cone (untreated)}] and the x-axis represent the different patient samples. (1B-D) Line graphs showing dose-dependent depletion in (1B) an exemplary highly depleted sample, (1C) an exemplary moderately depleted sample and (1D) and exemplary non-depleted sample. The y-axis represents the concentration of the free anti-AChR antibody in the sample in nM, and the x-axis shows the concentration of Alpha-ECD used for depleting the sample in nM. (1E) A bar graph of total antibody concentration in the serum samples used in 1A. Grey line indicates the clinical cutoff for disease. (1F) Diagram of the method of determining the percentage of AChR fragment-specific antibodies present in a sample by using a depletion assay. (1G) Dot plot showing the correlation between % Alpha binding and Anti-AChR titer.

FIGS. 2A-B: (2A) A contour plot showing for each serum sample of 41 MG patients the percentage of the relative anti-AChR antibodies that was detected against each AChR subunit (ε/δ/γ/β/α). (2B) A bar graph of the percent blocking for various subunits or combinations of subunits in the serum samples from 80 randomly tested MG patients. In addition to the average % depletion, the graph also shows the percent of patients that would have at least 50% or at least 75% depletion in each subunit or combination thereof.

FIGS. 3A-J: Diagrams of five possible embodiments of the four-chain therapeutic agent of the invention: (3A) shows a general embodiment of a molecule for treating AChR positive MG, (3B) shows an embodiment in which the four chains each comprise a different protein fragment, (3C) shows an embodiment in which the four protein fragments are all the same, (3D) shows an embodiment in which the two heavy chains are identical and the two light chains are identical, (3E) shows an embodiment in which the two heavy chains are different and the two light chains are identical, and (3F) shows an embodiment in which the two heavy chains contain the same protein fragment and the two light chains contain different protein fragments. (3G) Line graph of clinical score in rats inoculated with anti-AChR antibodies and then treated with various doses of the molecule of the invention. (3H) Kaplan Meier survival curve of rats inoculated with anti-AChR antibodies and then treated with various doses of the molecule of the invention. (3I) Line graph of clinical score in rats inoculated with anti-AChR antibodies and then treated with the molecule of the invention or two non-specific (NS) Fcs. (3J) Kaplan Meier survival curve of rats inoculated with anti-AChR antibodies and then treated with the molecule of the invention or the NS-Fcs.

FIGS. 4A-S: Diagrams of possible embodiments of the two-chain therapeutic agent of the invention: (4A) shows a general embodiment of a molecule with two heavy chains for treating AChR positive MG, (4B) shows embodiments in which at least one of the CH1, CH2 or CH3 domains has been excluded, (4C) shows an embodiment in which the two protein fragments are the same, (4D) shows an embodiment in which the two protein fragments are different, (4E) shows a general embodiment in which two tandem fragments are included in each heavy chain, (4F) shows the tandem fragment configuration in which all the subunits are the same, (4G) shows the tandem fragment configuration in which each heavy chain contains the same two different fragments, (4H) shows the tandem fragment configuration in which the two have chains contain different fragments that are not the same, (4I) shows a general embodiment in which two tandem fragments are included in one heavy chain while the other includes only one fragment, (4J) shows a general embodiment in which two tandem fragments are included in one heavy chain while the other is devoid of a fragment, (4K) shows the tandem configuration in which all three subunits are the same, (4L) shows the tandem configuration in which two of the three subunits are the same, (4M) shows the tandem configuration in which all three of the subunits are different, (4N) shows a general embodiment in which three tandem fragments are included in each heavy chain, (4O) shows the tandem configuration in which all six subunits are the same, (4P) shows the tandem configuration in which all three subunits on a chain are different but the two chains are the same, (4Q) shows the tandem configuration in which all three subunits on a chain are different and the two chains are different, (4R) shows a general embodiments in which three tandem fragments are included in one heavy chain and the other heavy chain includes 2, 1 or 0 fragments, and (4S) shows a general embodiment of a molecule with one heavy chain and one light chain.

FIGS. 5A-D: Diagrams of four possible embodiments of the three-chain therapeutic agent of the invention: (5A) shows a general embodiment of a molecule with two heavy chains and one light chain, (5B) shows an embodiment in which the three protein fragments are the same, (5C) shows an embodiment in which each of the protein fragments are different, (5D) shows an embodiment in which two of the protein fragments are the same and the third is different.

FIG. 6: Diagram of an embodiment of a four-chain therapeutic agent for treating AChR positive MG of the invention similar to those shown in FIG. 3 but in which the four chains each comprise a different protein fragment and a different immunoglobulin scaffold which promotes formation of the four-chain molecule.

FIGS. 7A-B: (7A-B) Diagrams of generic embodiments of the four-chain therapeutic agent of the invention: (7A) shows a generic embodiment of four chains in which two contain chains contain two dimerization domain and two chains contain a single dimerization domain, and (7B) shows an embodiment with optional linkers separating the various domains and fragments.

FIGS. 8A-E: Diagrams of single chain therapeutic agents of the invention: (8A) shows an embodiment of a single chain molecule containing fragments from two different AChR subunits, (8B) shows an embodiments of a single chain molecule containing fragments from three different AChR subunits, (8C) shows an embodiments of a single chain molecule containing fragments from four different AChR subunits, (8D) shows an embodiments of a single chain molecule containing fragments from two different AChR subunits and a heavy chain constant region, and (8E) shows the single chain molecules of 8A-D with amino acid (AA) linkers separating various domains.

FIG. 9: Bar graph of average percent depletion of AChR specific autoantibodies from MG sera contacted with the various molecules of the invention. To irrelevant extracellular domain constructs (CRD-239 and CRD-241) were used as negative controls. Each molecule was contacted with at least 17 different patient sera samples.

FIGS. 10A-D: Dot plots comparing the depletion rates produced by pairs of molecules: (10A) CRD-101 and CRD-269, (10B) CRD-101 and CRD-642, (10C) CRD-104 and CRD-391, and (10D) CRD-103 and CRD-382.

FIG. 11: Bar graph of fluorescent increase indicating binding of ECD tetramers to hybridoma cells. MFI fold change from background values were calculated by dividing ECD tetramer binding MFI of any hybridoma by the negative control background MFI. Hybridoma 204-4 was used as a negative control and tetramers CRD-233 and CRD-242 were also used as negative controls.

FIG. 12: Histogram of binding of ECD tetramers to a negative control B cell hybridoma line.

FIG. 13A-C: Bar graphs of binding of an alpha-gamma combination molecule tetramers to various B cell hybridoma cell lines: (13A) binding of CRD-506 to anti-alpha hybridoma and anti-gamma hybridoma, (13B) binding of CRD-509 and CRD-600 to anti-alpha hybridoma and (13C) binding of CRD-509 and CRD-600 to anti-gamma hybridoma and to negative control hybridomas.

FIG. 14: Bar graph of binding of various alpha subunit containing molecules to an anti-alpha hybridoma.

FIGS. 15A-C: Bar graphs of binding of (15A) an alpha-gamma molecule, (15B) a gamma-delta molecule, and (15C) an alpha-gamma molecule with an IgG4 Fc to various hybridomas.

### DETAILED DESCRIPTION OF THE INVENTION

The present invention, in some embodiments, provides compositions comprising a fragment of a first human receptor target of myasthenia gravis autoantibodies or an analog or derivative thereof and a fragment of a second human protein receptor of myasthenia gravis autoantibodies or an analog or derivative thereof. Protein complexes comprising at least two polypeptide chains wherein a first chain comprises a fragment of a first human protein target of myasthenia gravis autoantibodies or an analog or derivative thereof and a first dimerization domain and a second chain comprises a fragment of a second human protein target of myasthenia gravis autoantibodies or an analog or derivative thereof and a second dimerization domain capable of dimerizing with the first dimerization domain are also provided. The present invention further concerns pharmaceutical composition comprising the compositions and/or protein complexes, nucleic acids encoding the polypeptides of the compositions and/or protein complexes and methods of treatment and determining suitability for treatment using the compositions and/or protein complexes; as well as methods of producing the compositions and/or protein complexes.

By a first aspect, there is provided a protein comprising a first protein target of myasthenia gravis autoantibodies or an analog or derivative thereof.

By another aspect, there is provided a composition comprising a fragment of a first protein target of myasthenia gravis autoantibodies or an analog or derivative thereof and a fragment of a second protein target of myasthenia gravis autoantibodies or an analog or derivative thereof.

By another aspect, there is provided a protein comprising a fragment of a first protein target of myasthenia gravis autoantibodies or an analog or derivative thereof and a fragment of a second protein target of myasthenia gravis autoantibodies or an analog or derivative thereof.

By another aspect, there is provided a protein complex comprising at least two polypeptide chains, wherein a first polypeptide chain comprises a fragment of a first protein target of myasthenia gravis autoantibodies or an analog or derivative thereof and a first dimerization domain and a second polypeptide chain comprising a fragment of a second protein target of myasthenia gravis autoantibodies or an analog or derivative thereof and second dimerization domain.

In some embodiments, the protein is for use in treating myasthenia gravis. In some embodiments, the polypeptide is for use in treating myasthenia gravis. In some embodiments, the polypeptide chain is for use in treating myasthenia gravis. In some embodiments, the protein complex is for use in treating myasthenia gravis. In some embodiments, the composition is for use in treating myasthenia gravis. In some embodiments, the protein is a therapeutic agent. In some embodiments, the polypeptide is a therapeutic agent. In some embodiments, the polypeptide chain is a therapeutic agent. In some embodiments, the protein complex is a therapeutic agent.

In some embodiments, the composition comprises a protein complex comprising at least two polypeptide chains, wherein a first polypeptide chain comprises a fragment of a first protein target of myasthenia gravis autoantibodies or an analog or derivative thereof and a first dimerization domain and a second polypeptide chain comprising a fragment of a second protein target of myasthenia gravis autoantibodies or an analog or derivative thereof and second dimerization domain. In some embodiments, the composition comprises a protein complex of the invention. In some embodiments, the composition comprises a protein of the invention. In some embodiments, the protein is a recombinant protein. In some embodiments, the protein is a fusion protein.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the protein complex is an immunoglobulin (Ig)-like complex. In some embodiments, the protein complex comprises an Ig-like scaffold. In some embodiments, the protein complex comprises an Ig-like backbone. In some embodiments, the protein complex is an Ig Fc-fusion complex. In some embodiments, the composition is devoid of an antibody variable domain. In some embodiments, the protein complex is devoid of an antibody variable domain. In some embodiments, the composition is devoid of a variable domain. In some embodiments, the protein complex is devoid of a variable domain. In some embodiments, the first chain is devoid of a variable domain. In some embodiments, the second chain is devoid of a variable domain. In some embodiments, the protein complex is a multi-chain complex. In some embodiments, the composition is a therapeutic composition. In some embodiments, the protein complex is a therapeutic complex. In some embodiments, the composition is for use in a therapeutic method. In some embodiments, the protein complex is for use in a therapeutic method. In some embodiments, the composition is for use in production of a medicament. In some embodiments, the protein complex is for use in the production of a medicament. In some embodiments, the composition is for use in treating myasthenia gravis. In some embodiments, the protein complex is for use in treating myasthenia gravis. In some embodiments, the protein complex is for use in diagnosing myasthenia gravis. In some embodiments, the protein complex is for use in determining appropriate treatment in myasthenia gravis. In some embodiments, the protein complex is for use in characterizing the serological response in myasthenia gravis. In some embodiments, the protein complex is for use in determining the AChR antibody titer in myasthenia gravis.

As used herein, the term “polypeptide chain” refers to a polymer of amino acids linked by peptide bonds from an amino terminus (N-terminus) to a carboxyl terminus (C-terminus). In some embodiments, the polypeptide chain is a recombinant polypeptide. In some embodiments, a polypeptide chain comprises at least 5, 10, 15, 20, 25, 30, 35, 40, 45, 50, 55, 60, 65, 70, 75, 80, 85, 90, 100, 150, 200, 250, 300, 350, 400, 450 or 500 amino acids. Each possibility represents a separate embodiment of the invention. In some embodiments, a polypeptide chain comprises at most 300, 350, 400, 450, 500, 550, 600, 650, 700, 750, 800, 850, 900, 950, 1000, 1250, 1500, 1750, 2000, 2250, 2500, 2750, 3000, 3250, 3500, 3750, 4000, 4250, 4500, 4750, or 5000 amino acids. Each possibility represents a separate embodiment of the invention.

As used herein, the term “recombinant polypeptide” refers to a protein which is coded for by a recombinant DNA and is thus not naturally occurring. In some embodiments, the protein complex is not naturally occurring. In some embodiments, the polypeptide chain is not naturally occurring. In some embodiments, the recombinant polypeptide is a synthetic polypeptide. The term “recombinant DNA” refers to DNA molecules formed by laboratory methods. Generally, this recombinant DNA is in the form of a vector, plasmid or virus used to express the recombinant protein in a cell. Production of recombinant proteins by cellular expression is well known in the art and any method of recombinant protein expression may be used to produce the polypeptide of the invention. Cell free expression systems for recombinant protein production may also be employed.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, a nucleic acid molecule of the invention is expressed in a cell to produce a polypeptide of the invention. In some embodiments, a nucleic acid complex of the invention is expressed in a cell to produce a protein complex of the invention. In some embodiments, the RNA is a vector.

Expressing of a DNA sequence or an RNA within a cell is well known to one skilled in the art. It can be carried out by, among many methods, transfection, viral infection, or direct alteration of the cell's genome. In some embodiments, the DNA sequence is in an expression vector such as plasmid or viral vector. In some embodiments, a Kozak sequence is inserted upper stream of the transcription initiating codon. In some embodiments, the Kozak sequence enhances the amount of protein expresses.

In some embodiments, the protein complex comprises at least two polypeptide chains. In some embodiments, the protein complex comprises at least three polypeptide chains. In some embodiments, the protein complex comprises at least four polypeptide chains. In some embodiments, the protein complex comprises or consists of two polypeptide chains. In some embodiments, the protein complex comprises or consists of three polypeptide chains. In some embodiments, the protein complex comprises or consists of four chains. In some embodiments, the polypeptide chains are the same. In some embodiments, the polypeptide chains are different. In some embodiments, at least two of the polypeptide chains are the same. In some embodiments, at least two of the polypeptide chains are different.

It will be understood by a skilled artisan that the ECDs of the various subunits interact to form the full receptor and thus even without a dimerization domain can form a protein complex. In some embodiments, the protein complex comprises at least two proteins. In some embodiments, the protein complex comprises at least three proteins. In some embodiments, the protein complex comprises at least four proteins.

### Proteins

In some embodiments, the protein is a mammalian protein. In some embodiments, the mammal is a human. In some embodiments, the protein is a transmembrane protein. In some embodiments, the protein is a cell surface protein. In some embodiments, the protein is a receptor. In some embodiments, the protein is a subunit in a receptor. In some embodiments, the protein is a cell surface protein. In some embodiments, the cell surface protein is an integral membrane protein. In some embodiments, the cell surface protein is a plasma membrane embedded protein. In some embodiments, the cell surface protein is a membrane anchored protein. In some embodiments, the protein is a myasthenia gravis-associated protein. In some embodiments, the protein is a synthetic protein. In some embodiments, the protein is a naturally occurring protein. In some embodiments, the protein is a target of myasthenia gravis autoantibodies. In some embodiments, the protein is selected from AChRa, AChRb, AChRg, AChRd, and AChRe.

As used herein, the term “receptor” refers to a protein expressed on the surface of a cell that is capable of binding a ligand. In some embodiments, a receptor is a protein capable of transducing a signal to the cytoplasm of the cell. In some embodiments, a receptor comprises a ligand binding domain. In some embodiments, a receptor comprises a transmembrane domain. In some embodiments, a receptor comprises an intracellular domain.

In some embodiments, the fragment comprises an extracellular domain (ECD) of the protein. In some embodiments, the fragment comprises the entire extracellular domain or a variant thereof. In some embodiments, the fragment consists of the entire extracellular domain or a variant thereof. In some embodiments, a variant is a mutant. In some embodiments, a variant comprises a replacement of a portion of the extracellular domain. In some embodiments, the fragment comprises a fragment of an extracellular domain of the protein. In some embodiments, the fragment consists of an extracellular domain of the protein. In some embodiments, the fragment consists of a fragment of an extracellular domain of the protein. In some embodiments, the fragment comprises a transmembrane domain of the protein. In some embodiments, the fragment is devoid of a transmembrane domain of the protein. In some embodiments, the fragment is devoid of an intracellular domain of the protein. In some embodiments, the chain is devoid of a transmembrane domain. In some embodiments, the chain is devoid of an intracellular domain. In some embodiments, the fragment includes a sequence from a homologous human protein. In some embodiments, the fragment includes a sequence from a homologous non-human protein. In some embodiments, the fragment includes mutations in the human protein.

In some embodiments, the fragment comprises at least 5 amino acids of the protein. In some embodiments, the fragment comprises at least 10 amino acids of the protein. In some embodiments, the fragment comprises at least 5, 10 15, 20, 25, 30, 35, 40, 45, 50, 55, 60, 65, 70, 75, 80, 85, 90, 95 or 100 amino acids. Each possibility represents a separate embodiment of the invention. In some embodiments, amino acids of the protein are consecutive amino acids of the protein. In some embodiments, the fragment comprises less than 100% of the protein. In some embodiments, the fragment comprises less than 100% of an extracellular domain of the protein. In some embodiments, the fragment comprises less than 100, 99, 97, 95, 90, 85, 80, 75, 70, 65, 60, 55 or 50% of the protein. Each possibility represents a separate embodiment of the invention. In some embodiments, the fragment comprises less than 100, 99, 97, 95, 90, 85, 80, 75, 70, 65, 60, 55 or 50% of an extracellular domain of the protein. Each possibility represents a separate embodiment of the invention. In some embodiments, the fragment comprises between 5-500, 5-250, 5-100, 5-50, 10-500, 10-250, 10-100, 10-50, 20-500, 20-250, 20-200, 20-50, 25-500, 25-250, 25-100, 25-50, 50-500, 50-250, 50-100, 100-500, or 100-250 amino acids. Each possibility represents a separate embodiment of the invention. In some embodiments, a fragment comprises at most 20, 30, 40, 50, 60, 70, 75, 80, 90, 100, 110, 120, 125, 130,140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 550, 600, 650, 700, 750, 800, 850, 900, 950 or 1000 amino acids. Each possibility represents a separate embodiment of the invention.

In some embodiments, a variant comprises at least 70, 75, 80, 85, 90, 92, 95, 97, or 99% homology or identity. Each possibility represents a separate embodiment of the invention. In some embodiments, a variant comprises at least 85% homology or identity. In some embodiments, a variant comprises at least 90% homology or identity. In some embodiments, a variant comprises at least 92% homology or identity. In some embodiments, a variant comprises at least 95% homology or identity. In some embodiments, a variant comprises at least 97% homology or identity. In some embodiments, a variant comprises at least 99% homology or identity. In some embodiments, a variant is a mutant.

In some embodiments, the chain comprises at least one fragment. In some embodiments, the chain comprises at least two fragments. In some embodiments, the fragments are separated by a linker. In some embodiments, the linker is an amino acid linker. In some embodiments, the linker comprises at least one amino acid. In some embodiments, the linker is a flexible linker. In some embodiments, the linker comprises increased solubility as compared to a region of the protein excluded from the chain. In some embodiments, a region of the protein is replaced by a region of protein that is not the protein. In some embodiments, the replacement region comprises increased solubility as compared to the region of the protein that has been replaced. In some embodiments, the replacement region comprises increased protein stability as compared to the region of the protein that has been replaced.

In some embodiments, the protein is a target of antibodies. As used herein, the term “antibody” includes all classes of IgA, IgD, IgE, IgG and IgM and also includes all subclasses thereof. In some embodiments, the antibody is a circulating antibody. In some embodiments, the antibody is a naturally occurring antibody. In some embodiments, the antibodies are autoantibodies.

As used herein, the term “autoantibodies” refers to antibodies generated by a subject's own immune system against at least one of the subject's own proteins. In some embodiments, an autoantibody is an autoreactive antibody. In some embodiments, autoantibodies target self-antigens. Self-antigens are also known as autoantigens. In some embodiments, the autoantibodies are associated with myasthenia gravis. In some embodiments, the autoantibodies characterize myasthenia gravis. In some embodiments, the autoantibodies are autoantibodies of myasthenia gravis. In some embodiments, autoantibodies are generated by auto-reactive B cells. In some embodiments, the protein is an antigen of the antibodies. In some embodiments, the fragment comprises an antigen of the antibodies. In some embodiments, the fragment comprises at least one antigen of the antibodies. In some embodiments, the fragment comprises at least two antigens of the antibodies. In some embodiments, the fragment comprises at least 1, 2, 3, 4, 5, 6, 7, 8, 9 or 10 antigens of the antibodies. Each possibility represents a separate embodiment of the invention. In some embodiments, an antigen of the antibodies is an autoantigen. In some embodiments, the antigen is an epitope. In some embodiments, the antigen includes at least one epitope. In some embodiments, an epitope comprises at least 5 amino acids. In some embodiments, an epitope comprises 5-6 amino acids. In some embodiments, an epitope comprises 5-10 amino acids. In some embodiments, an epitope is a simple epitope. In some embodiments, a simple epitope is a linear epitope. In some embodiments, an epitope is a complex epitope. In some embodiments, a complex epitope is a 3D epitope. In some embodiments, a complex epitope is a discontinuous epitope. In some embodiments, a discontinuous epitope comprises at least two discontinuous sections of amino acids that combine to form an epitope. In some embodiments, a linker sequence is between the two sections of the epitope.

As used herein, the term “analog” includes any peptide having an amino acid sequence substantially identical to the sequence of the protein but in which one or more residues have been conservatively substituted with a functionally similar residue. In some embodiments, an analog displays similar functionality to the original protein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention. In some embodiments, the substitution is outside of an antigenic region of the protein. In some embodiments, the substitution is outside an epitope of the antibodies. In some embodiments, the analog is still a target of the antibodies. In some embodiments, the analog retains binding of autoantibodies. An analog may have deletions or mutations that result in an amino acids sequence that is different than the canonical amino acid sequence of protein. Further, an analog may be analogous to a fragment of the protein, however, in such a case the fragment must comprise at least 50 consecutive amino acids of protein or at least one epitope of the antibodies. In some embodiments, an analog is an analog to the canonical sequence of the protein.

In some embodiments, an analog to the protein comprises an amino acid sequence with at least 60%, at least 65%, at least 70%, at least 75%, at least 80%, at least 85%, at least 90%, at least 95%, at least 99% homology to the canonical amino acid sequence of the protein. Each possibility represents a separate embodiment of the invention. In some embodiments, an analog to the protein comprises an amino acid sequence with at least 60%, at least 65%, at least 70%, at least 75%, at least 80%, at least 85%, at least 90%, at least 95%, at least 99% identity to the canonical amino acid sequence of the protein. Each possibility represents a separate embodiment of the invention. In some embodiments, the analog comprises at least one substitution. In some embodiments, an analog comprises at least 1, 2, 3, 4, 5, 6, 7, 8, 9, or 10 substitutions. Each possibility represents a separate embodiment of the invention. In some embodiments, substitution is a mutation of the canonical sequence.

The term “derivative” as used herein, refers to any polypeptide that is based off the protein and still comprises retains binding of the antibodies. A derivative is not merely a fragment of the protein, nor does it have amino acids replaced or removed (an analog), rather it may have additional modification made to the protein, such as post-translational modification. Further, a derivative may be a derivative of a fragment of the protein, however, in such a case the fragment must comprise at least 50 consecutive amino acids of the protein or at least one epitope of the antibodies. In some embodiments, the derivative is a derivative of a canonical sequence of the protein.

In some embodiments, a derivative to the protein comprises an amino acid sequence with at least 60%, at least 65%, at least 70%, at least 75%, at least 80%, at least 85%, at least 90%, at least 95%, at least 99% homology to the canonical amino acid sequence of the protein. Each possibility represents a separate embodiment of the invention. In some embodiments, a derivative to the protein comprises an amino acid sequence with at least 60%, at least 65%, at least 70%, at least 75%, at least 80%, at least 85%, at least 90%, at least 95%, at least 99% identity to the canonical amino acid sequence of the protein. Each possibility represents a separate embodiment of the invention.

Canonical amino acid sequences of known proteins are well known in the art. They can be found in a variety of databases, including UniProt, NCBI, and the UCSC Genome Browser. Any sequence accepted as a canonical sequence may be employed. For a non-limiting example, human acetylcholine receptor subunit alpha is encoded by the CHRNA1 gene, its canonical nucleic acid sequence can be found in Entrez gene 1134, its canonical protein coding mRNA sequence can be found in NM_001039523 and NM_000079, its canonical amino acid sequence can be found in NP_000070 and NP_031415 and UniProt number P02708. In some embodiments, a canonical sequence is a sequence identical to the sequence present in at least 50, 60, 70, 75, 80, 90, 95, 97, or 99 percent of a population. Each possibility represents a separate embodiment of the invention. In some embodiments, a canonical sequence is a sequence identical to the most prevalent sequence present in a population. In some embodiments, the population is a disease population. In some embodiments, the population is a population with the autoimmune disease.

In some embodiments, the protein is acetylcholine receptor (AChR). In some embodiments, the protein is an acetylcholine receptor subunit. In some embodiments, the subunit is the alpha subunit. In some embodiments, the subunit is the beta subunit. In some embodiments, the subunit is the gamma subunit. In some embodiments, the subunit is the delta subunit. In some embodiments, the subunit is the epsilon subunit. In some embodiments, the subunit is selected from the alpha, beta, gamma, delta and epsilon subunits.

In some embodiments, the protein is the acetylcholine receptor alpha subunit (AChRa). In some embodiments, AChRa is encoded by the gene CHRNA1. In some embodiments, the AChRa is human AChRa. In some embodiments, CHRNA1 is identified by Entrez gene #1134. In some embodiments, AChRa is identified by UniProt ID P02708. In some embodiments, AChRa is identified by UniProt ID P020708.1 or P020708.2. In some embodiments, AChRa is identified by UniProt ID P020708.1. In some embodiments, AChRa is identified by UniProt ID P020708.2. In some embodiments, CHRNA1 comprises or consists of the nucleotide sequence provided in NM_001039523 or NM_000079. In some embodiments, AChRa comprises or consists of an amino acid sequence provided in NP_000070 or NP_001034612. In some embodiments, a canonical amino acid sequence of an extracellular domain of AChRa comprises or consists of

(SEQ ID NO: 1)
SEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADG
DFAIVKFTKVLLQYTGHITWTPPAIFKSYCEIIVTHFPFDEQNCSMKLG
TWTYDGSVVAINPESDQPDLSNFMESGEWVIKESRGWKHSVTYSCCPDT
PYLDITYHFVMQRLP.

In some embodiments, a canonical amino acid sequence of an extracellular domain of AChRa comprises or consists of SEHETRLVAKLFKDYSSVVRPVEDHRQVVEVTVGLQLIQLINVDEVNQIVTTNVRL KQGDMVDLPRPSCVTLGVPLFSHLQNEQWVDYNLKWNPDDYGGVKKIHIPSEKI WRPDLVLYNNADGDFAIVKFTKVLLQYTGHITWTPPAIFKSYCEIIVTHFPFDEQNC SMKLGTWTYDGSVVAINPESDQPDLSNFMESGEWVIKESRGWKHSVTYSCCPDTP YLDITYHFVMQRLP (SEQ ID NO: 2). In some embodiments, the extracellular domain is devoid of a signal peptide. In some embodiments, the extracellular domain further comprises a signal peptide. In some embodiments, the AChRa signal peptide comprises or consists of MEPWPLLLLFSLCSAGLVLG (SEQ ID NO: 3). In some embodiments, the AChRa signal peptide comprises or consists of MFMCLEGGEKNLTVLVSSAVSAGLVLG (SEQ ID NO: 61).

In some embodiments, the protein is the acetylcholine receptor beta subunit (AChRb). In some embodiments, AChRb is encoded by the gene CHRNB1. In some embodiments, the AChRb is human AChRb. In some embodiments, CHRNB1 is identified by Entrez gene #1140. In some embodiments, AChRb is identified by UniProt ID P11230. In some embodiments, CHRNB1 comprises or consists of the nucleotide sequence provided in NM_000747. In some embodiments, AChRb comprises or consists of an amino acid sequence provided in NP_000738. In some embodiments, a canonical amino acid sequence of an extracellular domain of AChRb comprises or consists of SEAEGRLREKLFSGYDSSVRPAREVGDRVRVSVGLILAQLISLNEKDEEMSTKVYL DLEWTDYRLSWDPAEHDGIDSLRITAESVWLPDVVLLNNNDGNFDVALDISVVVS SDGSVRWQPPGIYRSSCSIQVTYFPFDWQNCTMVFSSYSYDSSEVSLQTGLGPDGQ GHQEIHIHEGTFIENGQWEIIHKPSRLIQPPGDPRGGREGQRQEVIFYLIIRRKP (SEQ ID NO: 4). In some embodiments, the extracellular domain is devoid of a signal peptide. In some embodiments, the extracellular domain further comprises a signal peptide. In some embodiments, the AChRb signal peptide comprises or consists of MTPGALLMLLGALGAPLAPGVRG (SEQ ID NO: 5).

In some embodiments, the protein is the acetylcholine receptor gamma subunit (AChRg). In some embodiments, AChRg is encoded by the gene CHRNG. In some embodiments, the AChRg is human AChRg. In some embodiments, CHRNG is identified by Entrez gene #1146. In some embodiments, AChRg is identified by UniProt ID P07510. In some embodiments, CHRNG comprises or consists of the nucleotide sequence provided in NM_005199. In some embodiments, AChRg comprises or consists of an amino acid sequence provided in NP_005190. In some embodiments, a canonical amino acid sequence of an extracellular domain of AChRg comprises or consists of RNQEERLLADLMQNYDPNLRPAERDSDVVNVSLKLTLTNLISLNEREEALTTNVW IEMQWCDYRLRWDPRDYEGLWVLRVPSTMVWRPDIVLENNVDGVFEVALYCNV LVSPDGCIYWLPPAIFRSACSISVTYFPFDWQNCSLIFQSQTYSTNEIDLQLSQEDGQ TIEWIFIDPEAFTENGEWAIQHRPAKMLLDPAAPAQEAGHQKVVFYLLIQRKP (SEQ ID NO: 6). In some embodiments, the extracellular domain is devoid of a signal peptide. In some embodiments, the extracellular domain further comprises a signal peptide.

In some embodiments, the AChRg signal peptide comprises or consists of MHGGQGPLLLLLLLAVCLGAQG (SEQ ID NO: 7).

In some embodiments, the protein is the acetylcholine receptor delta subunit (AChRd). In some embodiments, AChRd is encoded by the gene CHRND. In some embodiments, the AChRd is human AChRd. In some embodiments, CHRND is identified by Entrez gene #1144. In some embodiments, AChRd is identified by UniProt ID Q07001. In some embodiments, CHRND comprises or consists of the nucleotide sequence provided in NM_000751, NM_001256657, NM_001311195, or NM_001311196. In some embodiments, AChRd comprises or consists of an amino acid sequence provided in NP_000742, NP_001243586, NP_001298124 or NP_001298125. In some embodiments, a canonical amino acid sequence of an extracellular domain of AChRd comprises or consists of LNEEERLIRHLFQEKGYNKELRPVAHKEESVDVALALTLSNLISLKEVEETLTTNV WIEHGWTDNRLKWNAEEFGNISVLRLPPDMVWLPEIVLENNNDGSFQISYSCNVL VYHYGFVYWLPPAIFRSSCPISVTYFPFDWQNCSLKFSSLKYTAKEITLSLKQDAKE NRTYPVEWIIIDPEGFTENGEWEIVHRPARVNVDPRAPLDSPSRQDITFYLIIRRKP (SEQ ID NO: 8). In some embodiments, the extracellular domain is devoid of a signal peptide. In some embodiments, the extracellular domain further comprises a signal peptide. In some embodiments, the AChRd signal peptide comprises or consists of MEGPVLTLGLLAALAVCGSWG (SEQ ID NO: 9).

In some embodiments, the protein is the acetylcholine receptor epsilon subunit (AChRe). In some embodiments, AChRe is encoded by the gene CHRNE. In some embodiments, the AChRe is human AChRe. In some embodiments, CHRNE is identified by Entrez gene #1145. In some embodiments, AChRe is identified by UniProt ID Q04844. In some embodiments, CHRNE comprises or consists of the nucleotide sequence provided in NM_000080. In some embodiments, AChRe comprises or consists of an amino acid sequence provided in NP_000071. In some embodiments, a canonical amino acid sequence of an extracellular domain of AChRe comprises or consists of KNEELRLYHHLFNNYDPGSRPVREPEDTVTISLKVTLTNLISLNEKEETLTTSVWIGI DWQDYRLNYSKDDFGGIETLRVPSELVWLPEIVLENNIDGQFGVAYDANVLVYEG GSVTWLPPAIYRSVCAVEVTYFPFDWQNCSLIFRSQTYNAEEVEFTFAVDNDGKTI NKIDIDTEAYTENGEWAIDFCPGVIRRHHGGATDGPGETDVIYSLIIRRKP (SEQ ID NO: 10). In some embodiments, the extracellular domain is devoid of a signal peptide. In some embodiments, the extracellular domain further comprises a signal peptide. In some embodiments, the AChRe signal peptide comprises or consists of MARAPLGVLLLLGLLGRGVG (SEQ ID NO: 11).

In some embodiments, the signal peptide is a signal peptide of an antibody chain. In some embodiments, the single peptide is of an antibody heavy chain. In some embodiments, the signal peptide is of an antibody light chain. In some embodiments, the signal peptide is of the Kappa light chain. In some embodiments, the signal peptide is of the Lambda light chain. In some embodiments, the heavy chain signal peptide comprises MEWSWVFLFFLSVTTGVHS (SEQ ID NO: 70). In some embodiments, the heavy chain signal peptide consists of SEQ ID NO: 70. In some embodiments, the heavy chain signal peptide comprises MEFGLSWLFLVAILKGVQC (SEQ ID NO: 14). In some embodiments, the heavy chain signal peptide consists of SEQ ID NO: 14. In some embodiments, the light chain signal peptide comprises MSVPTQVLGLLLLWLTDARC (SEQ ID NO: 71). In some embodiments, the light chain signal peptide consists of SEQ ID NO: 71. In some embodiments, the heavy chain signal peptide is the mouse heavy chain signal peptide and comprises MGWSCIILFLVATATGVHS (SEQ ID NO: 15). In some embodiments, the heavy chain signal peptide consists of SEQ ID NO: 15.

In some embodiments, the first protein and the second protein are the same protein. In some embodiments, the first and second proteins are the same proteins, and the fragments are different fragments. In some embodiments, the fragments are different fragments. In some embodiments, the fragments comprise or consist of different sequences. In some embodiments, the first and second proteins are different proteins.

In some embodiments, the protein or fragment comprises a mutation that increases solubility. In some embodiments, the protein or fragment comprises a mutation that increases stability of the protein or fragment. In some embodiments, the mutation is an insertion. In some embodiments, the protein is a surface protein and comprises a mutation that increases solubility. In some embodiments, the fragment is an extracellular domain of a surface protein and comprises an insertion that increases solubility. In some embodiments, the insertion is in place of a region of the protein or fragment. In some embodiments, a loop of the protein is replaced with a loop with higher solubility. In some embodiments, the loop of AChRa comprises or consists of CEIIVTHFPFDEQNC (SEQ ID NO: 39). In some embodiments, the loop of AChRb comprises or consists of CSIQVTYFPFDWQNC (SEQ ID NO: 40). In some embodiments, the loop of AChRg comprises or consists of CSISVTYFPFDWQNC (SEQ ID NO: 41). In some embodiments, the loop of AChRd comprises or consists of CPISVTYFPFDWQNC (SEQ ID NO: 42). In some embodiments, the loop of AChRe comprises or consists of CAVEVTYFPFDWQNC (SEQ ID NO: 43). In some embodiments, the loop with higher solubility comprises or consists of CDVSGVDTESGATNC (SEQ ID NO: 44). In some embodiments, the insert of higher solubility comprises or consists of SEQ ID NO: 44. In some embodiments, the insert of higher solubility comprises DVSGVDTESGAT (SEQ ID NO: 63). In some embodiments, AChR is mutated to increase solubility and stability. In some embodiments, the mutation is selected from V8E, W149R and V155A. In some embodiments, the mutation is mutation of at least two of V8E, W149R and V155A. In some embodiments, the mutation is mutation of all three of V8E, W149R and V155A. In some embodiments, AChRa comprises the mutation. In some embodiments, a non-AChRa acetylcholine receptor subunit comprises parallel mutations. In some embodiments, parallel mutation are mutations to amino acids with homology.

In some embodiments, the alpha subunit extracellular domain into which mutations are made comprises or consists of SEHETRLVAKLFKDYSSVVRPVEDHRQVVEVTVGLQLIQLINVDEVNQIVTTNVRL KQQWVDYNLKWNPDDYGGVKKIHIPSEKIWRPDLVLYNNADGDFAIVKFTKVLL QYTGHITWTPPAIFKSYCDVSGVDTESGATNCSMKLGTWTYDGSVVAINPESDQP DLSNFMESGEWVIKESRGWKHSVTYSCCPDTPYLDITYHFVMQRLP (SEQ ID NO: 131). In some embodiments, the beta subunit extracellular domain into which mutations are made comprises or consists of SEAEGRLREKLFSGYDSSVRPAREVGDRVRVSVGLILAQLISLNEKDEEMSTKVYL DLEWTDYRLSWDPAEHDGIDSLRITAESVWLPDVVLLNNNDGNFDVALDISVVVS SDGSVRWQPPGIYRSSCDVSGVDTESGATNCTMVFSSYSYDSSEVSLQTGLGPDG QGHQEIHIHEGTFIENGQWEIIHKPSRLIQPPGDPRGGREGQRQEVIFYLIIRRKP (SEQ ID NO: 132). In some embodiments, the gamma subunit extracellular domain into which mutations are made comprises or consists of RNQEERLLADLMQNYDPNLRPAERDSDVVNVSLKLTLTNLISLNEREEALTTNVW IEMQWCDYRLRWDPRDYEGLWVLRVPSTMVWRPDIVLENNVDGVFEVALYCNV LVSPDGCIYWLPPAIFRSACDVSGVDTESGATNCSLIFQSQTYSTNEIDLQLSQEDG QTIEWIFIDPEAFTENGEWAIQHRPAKMLLDPAAPAQEAGHQKVVFYLLIQRKP (SEQ ID NO: 133). In some embodiments, the delta subunit extracellular domain into which mutations are made comprises or consists of LNEEERLIRHLFQEKGYNKELRPVAHKEESVDVALALTLSNLISLKEVEETLTTNV WIEHGWTDNRLKWNAEEFGNISVLRLPPDMVWLPEIVLENNNDGSFQISYSCNVL VYHYGFVYWLPPAIFRSSCDVSGVDTESGATNCSLKFSSLKYTAKEITLSLKQDAK ENRTYPVEWIIIDPEGFTENGEWEIVHRPARVNVDPRAPLDSPSRQDITFYLIIRRKP (SEQ ID NO: 134). In some embodiments, the epsilon subunit extracellular domain into which mutations are made comprises or consists of

(SEQ ID NO: 135)
KNEELRLYHHLFNNYDPGSRPVREPEDTVTISLKVTLTNLISLNEKEET
LTTSVWIGIDWQDYRLNYSKDDFGGIETLRVPSELVWLPEIVLENNIDG
QFGVAYDANVLVYEGGSVTWLPPAIYRSVCDVSGVDTESGATNCSLIFR
SQTYNAEEVEFTFAVDNDGKTINKIDIDTEAYTENGEWAIDFCPGVIRR
HHGGATDGPGETDVIYSLIIRRKP.

By another aspect, there is provided a protein comprises any one of SEQ ID NO: 131-135.

In some embodiments, the protein comprises SEQ ID NO: 131. In some embodiments, the protein comprises SEQ ID NO: 132. In some embodiments, the protein comprises SEQ ID NO: 133. In some embodiments, the protein comprises SEQ ID NO: 134. In some embodiments, the protein comprises SEQ ID NO: 135. In some embodiments, the protein comprises a plurality of sequences selected from SEQ ID NO: 131-135. In some embodiments, a complex of the invention comprises a first chain comprising a sequence selected from SEQ ID NO: 131-135. In some embodiments, a complex of the invention comprises a second chain comprising a sequence selected from SEQ ID NO: 131-135. In some embodiments, a complex of the invention comprises a third chain comprising a sequence selected from SEQ ID NO: 131-135. In some embodiments, a complex of the invention comprises a fourth chain comprising a sequence selected from SEQ ID NO: 131-135. In some embodiments, the extracellular domain fragment is selected from SEQ ID NO: 131-135. In some embodiments, the extracellular domain variant is selected from SEQ ID NO: 131-135.

In some embodiments, the fragment comprises a ligand binding domain and further comprises a mutation that inhibits ligand binding. In some embodiments, the mutation is in the ligand binding domain. It will be understood by a skilled artisan that as the protein complex of the invention is meant to bind antibodies and B cells it would be advantageous not to bind the endogenous ligand present in the subject and thus leaves normal ligand levels available to bind to the endogenous receptor. In some embodiments, the protein is AChRa and the mutation is tyrosine 190 of SEQ ID NO: 1 or tyrosine 215 of SEQ ID NO: 2 to phenylalanine. In some embodiments, the protein is AChRa and the mutation is tyrosine 190 of SEQ ID NO: 1 to phenylalanine. In some embodiments, the protein is AChRa and the mutation is tyrosine 215 of SEQ ID NO: 2 to phenylalanine.

By another aspect, there is provided a protein comprising an extracellular domain of an acetylcholine receptor subunit comprising at least one mutation that decreases aggregation.

In some embodiments, the fragment comprises a mutation that decreases aggregation. In some embodiments, aggregation comprises auto-dimerization. In some embodiments, aggregation comprises multimerization. In some embodiments, the extracellular domain of an acetylcholine receptor subunit is the protein. In some embodiments, a mutation is a plurality of mutations. In some embodiments, a plurality is two. In some embodiments, a plurality is three.

In some embodiments, the protein is AChRa and the mutation is deletion of N141. In some embodiments, N141 is within SEQ ID NO: 1. In some embodiments, N141 is within SEQ ID NO: 131. In some embodiments, the protein is AChRa and the mutation is mutation of phenylalanine 100. In some embodiments, phenylalanine 100 is mutated to glycine (F100G). In some embodiments, phenylalanine 100 is mutated to tyrosine (F100Y). In some embodiments, phenylalanine 100 is mutated to isoleucine (F100I). In some embodiments, F100 is within SEQ ID NO: 1. In some embodiments, F100 is within SEQ ID NO: 131. In some embodiments, the protein is AChRa and the mutation is mutation of tryptophan 149.

In some embodiments tryptophan 149 is mutated to a charged amino acid. In some embodiments, tryptophan 149 is mutated to a negatively charged amino acid. In some embodiments, tryptophan 149 is mutated to glutamic acid (W149E). In some embodiments, tryptophan 149 is mutated to aspartic acid (W149D). In some embodiments, tryptophan 149 is mutated to a positively charged amino acid. In some embodiments, tryptophan 149 is mutated to lysine (W149K). In some embodiments, tryptophan 149 is mutated to arginine (W149R). In some embodiments, tryptophan 149 is mutated to histidine (W149H). In some embodiments, tryptophan 149 is mutated to glutamine (W149Q). In some embodiments, W149 is within SEQ ID NO: 1. In some embodiments, W149 is within SEQ ID NO: 131. In some embodiments, the protein is AChRa and the mutation is mutation of valine 155. In some embodiments, valine 155 is mutated to alanine (V155A). In some embodiments, valine 155 is mutated to isoleucine (V155I). In some embodiments, valine 155 is mutated to leucine (V155L). In some embodiments, V155 is within SEQ ID NO: 1. In some embodiments, V155 is within SEQ ID NO: 131. In some embodiments, the protein is AChRa and the mutation is mutation of tyrosine 93. In some embodiments, mutation of tyrosine 93 decreases alpha-gamma interactions. In some embodiments, the tyrosine 93 is mutated to any amino acid that decreases alpha to gamma interaction. In some embodiments, tyrosine 93 is mutated to phenylalanine (Y93F). In some embodiments, tyrosine 93 is mutated to a positively charged amino acid. In some embodiments, tyrosine 93 is mutated to histidine (Y93H). In some embodiments, tyrosine 93 is mutated to arginine (Y93R). In some embodiments, tyrosine 93 is mutated to lysine (Y93K). In some embodiments, Y93 is within SEQ ID NO: 1. In some embodiments, Y93 is within SEQ ID NO: 131.

In some embodiments, the mutation reduces oxidation of the protein. In some embodiments, the protein is AChRg and the mutation is mutation of methionine 84. The mutation is made, at least in part, to decrease methionine oxidation and increase shelf life. Mutation to any amino acid will produce this result. In some embodiments, mutation of methionine 84 reduces alpha-gamma interaction. In some embodiments, the methionine 84 is mutated to any amino acid that decreases alpha to gamma interaction. In some embodiments, methionine 84 is deleted. In some embodiments, methionine 84 is mutated to serine (M84S). In some embodiments, M84 is within SEQ ID NO: 6. In some embodiments, M84 is within SEQ ID NO: 133. In some embodiments, the protein is AChRg and the mutation is mutation of tyrosine 105. In some embodiments, mutation of tyrosine 105 reduces alpha-gamma interaction. In some embodiments, the tyrosine 105 is mutated to any amino acid that decreases alpha to gamma interaction. In some embodiments, tyrosine 105 is mutated to a charged amino acid. In some embodiments, tyrosine 105 is mutated to a negatively charged amino acid. In some embodiments, tyrosine 105 is mutated to glutamic acid (Y105E). In some embodiments, tyrosine 105 is mutated to aspartic acid (Y105D). In some embodiments, tyrosine 105 is mutated to a positively charged amino acid. In some embodiments, tyrosine 105 is mutated to arginine (Y105R). In some embodiments, tyrosine 105 is mutated to lysine (Y105K). In some embodiments, tyrosine 105 is mutated to histidine (Y105H). In some embodiments, Y105 is within SEQ ID NO: 6. In some embodiments, Y105 is within SEQ ID NO: 133. In some embodiments, the protein is AChRg and the mutation is mutation of tyrosine 117. In some embodiments, mutation of tyrosine 117 reduces alpha-gamma interaction. In some embodiments, the tyrosine 117 is mutated to any amino acid that decreases alpha to gamma interaction. In some embodiments, tyrosine 117 is mutated to a charged amino acid. In some embodiments, tyrosine 117 is mutated to a negatively charged amino acid. In some embodiments, tyrosine 117 is mutated to glutamic acid (Y117E). In some embodiments, tyrosine 117 is mutated to aspartic acid (Y117D). In some embodiments, tyrosine 117 is mutated to a positively charged amino acid. In some embodiments, tyrosine 117 is mutated to arginine (Y117R). In some embodiments, tyrosine 117 is mutated to lysine (Y117K). In some embodiments, tyrosine 117 is mutated to histidine (Y117H). In some embodiments, Y117 is within SEQ ID NO: 6. In some embodiments, Y117 is within SEQ ID NO: 133. In some embodiments, the mutation is a plurality of mutations and comprises at least two mutations selected from mutation of M84, Y105 and Y117. In some embodiments, the mutation is a plurality of mutations and comprises at least two mutations selected from M84S, Y105E and Y117E. In some embodiments, the mutation is a plurality of mutations and comprises at least two mutations selected from M84S, Y105E and Y117R. In some embodiments, the mutation is a plurality of mutations and comprises at all of M84S, Y105E and Y117E. In some embodiments, the mutation is a plurality of mutations and comprises all of M84S, Y105E and Y117R In some embodiments, the mutation is two mutations and comprises M84S and Y105E. In some embodiments, the mutation is two mutations and comprises Y117E and Y105E. In some embodiments, the mutation is two mutations and comprises Y117R and Y105E.

In some embodiments, the protein is AChRd and the mutation is mutation of cysteine 108. In some embodiments, cysteine 108 is mutated to any other amino acid. In some embodiments, cysteine 108 is deleted. It will be understood by a skilled artisan that the desire is to decrease aggregation by removing a free cysteine that could have formed a disulfide bond. As such any mutation is possible. In some embodiments, cysteine 108 is mutated to alanine (C108A). In some embodiments, cysteine 108 is mutated to isoleucine (C108I). In some embodiments, C108 is within SEQ ID NO: 8. In some embodiments, C108 is within SEQ ID NO: 134. In some embodiments, the protein is AChRd and the mutation is mutation of tyrosine 119. In some embodiments, mutation of tyrosine 119 reduces delta hydrophobicity. In some embodiments, the tyrosine 119 is mutated to any amino acid that decreases delta hydrophobicity. In some embodiments, tyrosine 119 is mutated to a positively charged amino acid. In some embodiments, tyrosine 119 is mutated to arginine (Y119R). In some embodiments, tyrosine 119 is mutated to lysine (Y119K). In some embodiments, tyrosine 119 is mutated to histidine (Y119H). In some embodiments, tyrosine 119 is mutated to a negatively charged amino acid. In some embodiments, tyrosine 119 is mutated to glutamic acid (Y119E). In some embodiments, tyrosine 119 is mutated to aspartic acid (Y119D). In some embodiments, Y119 is within SEQ ID NO: 8. In some embodiments, Y119 is within SEQ ID NO: 134. In some embodiments, the protein is AChRa and the mutation is deletion of N141. In some embodiments, N141 is within SEQ ID NO: 8. In some embodiments, N141 is within SEQ ID NO: 134. In some embodiments, the protein is AChRd and the mutation is mutation of leucine 151. In some embodiments, mutation of leucine 151 reduces subunit interaction. In some embodiments, the leucine 151 is mutated to any amino acid that decreases subunit interaction. In some embodiments, leucine 151 is deleted. In some embodiments, leucine 151 is mutated to a charged amino acid. In some embodiments, leucine 151 is mutated to a positively charged amino acid. In some embodiments, leucine 151 is mutated to an amino acid that is not positively charged. In some embodiments, leucine 151 is mutated to a negatively charged amino acid. In some embodiments, leucine 151 is mutated to glutamic acid (L151E). In some embodiments, leucine 151 is mutated aspartic acid (L151D). In some embodiments, L151 is within SEQ ID NO: 8. In some embodiments, L151 is within SEQ ID NO: 134. In some embodiments, the mutation is a plurality of mutations selected from mutation of C108, Y119, L151 and deletion of N141. In some embodiments, the mutation is a plurality of mutations selected from C108A, Y119R, L151E and deletion of N141. In some embodiments, the mutation is two mutations and comprises C108A and Y119R. In some embodiments, the mutation is two mutations and comprises C108A and L151E. In some embodiments, the mutation is two mutations and comprises C108A and deletion of N141. In some embodiments, the mutation is three mutations and comprises C108A, Y119R and L151E. In some embodiments, the mutation is three mutations and comprises C108A, Y119R and deletion of N141.

### Dimerization Domains

In some embodiments, dimerization domains are capable of dimerizing with each other. In some embodiments, the first dimerization domain is capable of dimerization with the second dimerization domain. In some embodiments, the first and second dimerization domains are capable of dimerizing with each other. In some embodiments, capable of dimerizing is configured to dimerize. In some embodiments, dimerization is under physiological conditions. In some embodiments, dimerization is within a bodily fluid. In some embodiments, the bodily fluid is blood. In some embodiments, the bodily fluid is plasma. In some embodiments, the bodily fluid is serum. In some embodiments, dimerization is within a subject. In some embodiments, dimerization is in vivo. In some embodiments, dimerization is in vitro.

As used herein, the term “dimerization domain” refers to an amino acid sequence that upon contacting another amino acid sequence (the other dimerization domain) binds to it to form a dimer. Dimerization domains are well known in the art, as many protein sequences are known to bind to each other. In some embodiments, dimerization comprises formation of a covalent bond between the dimerization domains. In some embodiments, dimerization comprises electrostatic binding. In some embodiments, dimerization does not comprise electrostatic binding. In some embodiments, dimerization is reversible. In some embodiments, dimerization is irreversible. In some embodiments, dimerization comprises a bond forming between the dimerization domains. In some embodiments, the bond is a chemical bond. In some embodiments, the bond is a disulfide bond. In some embodiments, the bond is a peptide bond. Examples of dimerization domain include the hinge domain of antibody heavy chains, the CH1/CL domains of antibody heavy/light chains, and the ECD domains of TCR alpha/beta to name but a few. Additionally, the upper hinge domain can be engineered with cysteine substitutions/mutations to serine in order to prevent dimerization. In some embodiments, the dimerization domain comprises or consists of the sequence EPKSSDKTHTCPPCP (SEQ ID NO: 63).

In some embodiments, the dimerization domain comprises or consists of an immunoglobulin (Ig) hinge domain. In some embodiments, an Ig hinge domain is a heavy chain hinge domain. In some embodiments, the Ig is a human Ig. In some embodiments, the immunoglobulin is elected from IgA, IgD, IgE, IgG and IgM. In some embodiments, the immunoglobulin is IgG. In some embodiments, the IgG is IgG1. In some embodiments, the IgG is IgG2. In some embodiments, the IgG is IgG3. In some embodiments, the IgG is selected from IgG1 and IgG3. In some embodiments, the IgG is IgG4. In some embodiments, the first and second dimerization domains are both Ig hinge domains. In some embodiments, the first and second dimerization domains are identical. In some embodiments, the first and second dimerization domains are at least 95% identical. In some embodiments, the first and second dimerization domains are at least 50, 55, 60, 65, 70, 75, 80, 85, 90, 95, 97, 99 or 100% identical. Each possibility represents a separate embodiment of the invention.

In some embodiments, the hinge domain comprises the amino acid sequence EPKSCDKTHTCPPCPAPELLGG (SEQ ID NO: 16). In some embodiments, the hinge domain consists of the amino acid sequence of SEQ ID NO: 16. In some embodiments, the IgG1 hinge comprises or consists of SEQ ID NO: 16. In some embodiments, the hinge domain comprises the amino acid sequence EPKCCVECPPCPAPPAAA (SEQ ID NO: 17). In some embodiments, the hinge domain consists of the amino acid sequence of SEQ ID NO: 17. In some embodiments, the IgG2 hinge comprises or consists of SEQ ID NO: 17. In some embodiments, the hinge domain comprises the amino acid sequence ESKYGPPCPPCPAPEFLGG (SEQ ID NO: 18). In some embodiments, the hinge domain consists of the amino acid sequence of SEQ ID NO: 18. In some embodiments, the IgG4 hinge comprises or consists of SEQ ID NO: 18. In some embodiments, the hinge domain comprises the amino acid sequence ELKTPLGDTTHTCPRCPEPKSCDTPPPCPRCPEPKSCDTPPPCPRCPEPKSCDTPPPC PRCPAPELLGGP (SEQ ID NO: 19). In some embodiments, the hinge domain consists of the amino acid sequence of SEQ ID NO: 19. In some embodiments, the IgG3 hinge comprises or consists of SEQ ID NO: 19. In some embodiments, the hinge domain comprises a CPXCP (SEQ ID NO: 20) motif. In some embodiments, the X in SEQ ID NO: 20 is selected from P and R. In some embodiments, SEQ ID NO: 20 is CPPCP (SEQ ID NO: 21). In some embodiments, SEQ ID NO: 20 is CPRCP (SEQ ID NO: 22). In some embodiments, the hinge domain comprises EPKSCDKTHTCPPCP (SEQ ID NO: 37). It will thus be understood that the hinge region can be considered to end after the CPXCP motif.

In some embodiments, the dimerization domain comprises or consists of an Ig CH1 domain. In some embodiments, the dimerization domain comprises or consists of an Ig heavy chain CH1 domain. In some embodiments, the dimerization domain comprises or consists of an Ig light chain. In some embodiments, the dimerization domain comprises or consists of a light chain CL domain. In some embodiments, the CL domain is a CL kappa domain. In some embodiments, the CL domain is a CL lambda domain. It is well known in the art that the CH1 domain of the Ig heavy chain dimerizes with the light chain CL domain. In some embodiments, the first dimerization domain comprises or consists of a CH1 domain, and the second dimerization domain comprises or consists of a CL domain. In some embodiments, the first and second dimerization domains both comprise a hinge domain. In some embodiments, the first and second dimerization domains do not both comprise a CH1 domain. In some embodiments, the first and second dimerization domains do not both comprise a CL domain. In some the first and second polypeptide chains do not both comprise a CH1 domain. In some the first and second polypeptide chains do not both comprise a CL domain.

In some embodiments, an Ig CH1 domain comprises of the amino acid sequence ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKV (SEQ ID NO: 23). In some embodiments, an Ig CH1 domain consists of SEQ ID NO: 23. In some embodiments, SEQ ID NO: 23 is the IgG1 CH1 domain. In some embodiments, an Ig CH1 domain comprises of the amino acid sequence ASTKGPSVFPLAPCSRSTSESTAALGCLVKDYFPEPVTVSWNSGALTSGVHTFPAV LQSSGLYSLSSVVTVPSSNFGTQTYTCNVDHKPSNTKVDKTV (SEQ ID NO: 24). In some embodiments, an Ig CH1 domain consists of SEQ ID NO: 24. In some embodiments, SEQ ID NO: 24 is the IgG2 CH1 domain. In some embodiments, an Ig CH1 domain comprises of the amino acid sequence ASTKGPSVFPLAPCSRSTSGGTAALGCLVKDYFPEPVTVSWNSGALTSGVHTFPAV LQSSGLYSLSSVVTVPSSSLGTQTYTCNVNHKPSNTKVDKRV (SEQ ID NO: 25). In some embodiments, an Ig CH1 domain consists of SEQ ID NO: 25. In some embodiments, SEQ ID NO: 25 is the IgG3 CH1 domain. In some embodiments, an Ig CH1 domain comprises of the amino acid sequence ASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRV (SEQ ID NO: 26). In some embodiments, an Ig CH1 domain consists of SEQ ID NO: 26. In some embodiments, SEQ ID NO: 26 is the IgG4 CH1 domain.

In some embodiments, an Ig CL Kappa domain comprises of the amino acid sequence AAPSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGEC (SEQ ID NO: 27). In some embodiments, an Ig CL Kappa domain consists of SEQ ID NO: 27. In some embodiments, an Ig CL Lambda domain comprises of the amino acid sequence GQPKANPTVTLFPPSSEELQANKATLVCLISDFYPGAVTVAWKADGSPVKAGVET TKPSKQSNNKYAASSYLSLTPEQWKSHRSYSCQVTHEGSTVEKTVAPTECS (SEQ ID NO: 28). In some embodiments, an Ig CL Lambda domain consists of SEQ ID NO: 28.

### Fc Region

In some embodiments, the composition comprises an Fc region. In some embodiments, an Fc region is an Fc domain. In some embodiments, an Fc region is an Fc fragment. In some embodiments, the first polypeptide chain comprises an Fc region. In some embodiments, the second polypeptide chain comprises an Fc region. In some embodiments, both the first and second polypeptide chains comprise an Fc region. In some embodiments, the Fc region is an Fc region of an antibody heavy chain. In some embodiments, the antibody heavy chain is a human antibody heavy chain. In some embodiments, the heavy chain is an IgG heavy chain. In some embodiments, the IgG is selected from IgG1, IgG2, IgG3 and IgG4. In some embodiments, the IgG is selected from IgG1 and IgG3. In some embodiments, the IgG is IgG1. In some embodiments, the IgG is IgG2. In some embodiments, the IgG is IgG3. In some embodiments, the IgG is IgG4.

In some embodiments, the Fc region is capable of inducing a cytotoxic effect. In some embodiments, the Fc domain comprises DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALP APIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSL SLSPGK (SEQ ID NO: 12). In some embodiments, the Fc domain consists of SEQ ID NO: 12. In some embodiments, the Fc domain of IgG1 comprises or consists of SEQ ID NO: 12. In some embodiments, the Fc domain comprises or consists of a sequence with at least 70, 75, 80, 85, 90, 93, 95, 97, or 99% homology to SEQ ID NO: 12. Each possibility represents a separate embodiment of the invention. In some embodiments, the Fc region is configured to induce a cytotoxic effect. In some embodiments, the cytotoxic effect is against a target cell. In some embodiments, the cytotoxic effect is upon binding. In some embodiments, the cytotoxic effect is against a cell bound by the protein complex. In some embodiments, the cytotoxic effect is against a cell binding the protein complex. In some embodiments, the cytotoxic effect is mediated by immune cell binding to the Fc region. In some embodiments, the cytotoxic effect is mediated by immune cell activation by the Fc region. In some embodiments, the cytotoxic effect is mediated by immune cell recruitment by the Fc region. In some embodiments, the immune cell is a T cell. In some embodiments, the immune cell is a natural killer (NK) cell. In some embodiments, the immune cell is a macrophage. In some embodiments, the T cell is a cytotoxic T cell. In some embodiments, the T cell is a CD8 positive T cell. In some embodiments, the Fc region induces antibody-dependent cell cytotoxicity (ADCC). In some embodiments, the Fc region induces complement-dependent cytotoxicity (CDC).

In some embodiments, the Fc region comprises an Ig CH2 domain. In some embodiments, the Fc region comprises an Ig heavy chain CH2 domain. In some embodiments, the Fc region comprises an Ig CH3 domain. In some embodiments, the Fc region comprises an Ig heavy chain CH3 domain. In some embodiments, the Fc region comprises or consists of both an Ig CH2 domain and Ig CH3 domain. In some embodiments, the Fc region comprises or consists of both an Ig heavy chain CH2 and an Ig heavy chain CH3 domain. In some embodiments, the first chain comprises a first portion of an Fc region and the second chain comprises a second portion of the Fc region. In some embodiments, the first portion comprises a CH2 domain, a CH3 domain or both. In some embodiments, the second portion comprises a CH2 domain, a CH3 domain or both. In some embodiments, interface of the first portion of an Fc region and the second portion of an Fc region produces a functional Fc region. In some embodiments, interface comprises contact. In some embodiments, interface comprises adjacent positioning. In some embodiments, interface comprises formation of the protein complex of the invention. In some embodiments, interface comprises dimerization of the first and second dimerization domains. In some embodiments, the CH2 domain is an Ig CH2 domain. In some embodiments the CH2 domain is a heavy chain CH2 domain. In some embodiments, the CH3 domain is an Ig CH3 domain.

In some embodiments, the CH3 domain is a heavy chain CH3 domain. In some embodiments, a CH2 domain comprises the amino acid sequence SVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAK (SEQ ID NO: 29). In some embodiments, a CH2 domain comprises the amino acid sequence DKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALP APIEKTISKAK (SEQ ID NO: 13). In some embodiments, the CH2 domain consists of SEQ ID NO: 29. In some embodiments, SEQ ID NO: 29 is the IgG1 CH2 domain. In some embodiments, a CH2 domain comprises the amino acid sequence SVFLFPPKPKDTLMISRTPEVTCVVVDVSHEDPEVQFNWYVDGVEVHNAKTKPRE EQFNSTFRVVSVLTVVHQDWLNGKEYKCKVSNKGLPAPIEKTISKTK (SEQ ID NO: 30). In some embodiments, the CH2 domain consists of SEQ ID NO: 30. In some embodiments, SEQ ID NO: 30 is the IgG2 CH2 domain. In some embodiments, a CH2 domain comprises the amino acid sequence SVFLFPPKPKDTLMISRTPEVTCVVVDVSQEDPEVQFNWYVDGVEVHNAKTKPRE EQFNSTYRVVSVLTVLHQDWLNGKEYKCKVSNKGLPSSIEKTISKAK (SEQ ID NO: 31). In some embodiments, the CH2 domain consists of SEQ ID NO: 31. In some embodiments, SEQ ID NO: 31 is the IgG4 CH2 domain. In some embodiments, a CH2 domain comprises the amino acid sequence SVFLFPPKPKDTLMISRTPEVTCVVVDVSHEDPEVQFKWYVDGVEVHNAKTKPRE EQYNSTFRVVSVLTVLHQDWLNGKEYKCKVSNKALPAPIEKTISKTK (SEQ ID NO: 32). In some embodiments, the CH2 domain consists of SEQ ID NO: 32. In some embodiments, SEQ ID NO: 32 is the IgG3 CH2 domain.

In some embodiments, a CH3 domain comprises the amino acid sequence GQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 33). In some embodiments, a CH3 domain comprises the amino acid sequence GQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 62). In some embodiments, the CH3 domain consists of SEQ ID NO: 33. In some embodiments, the CH3 domain consists of SEQ ID NO: 62. In some embodiments, SEQ ID NO: 33 is the IgG1 CH3 domain. In some embodiments, SEQ ID NO: 62 is the IgG1 CH3 domain. In some embodiments, the SEQ ID NO: 33 sequence is the sequence found predominantly is humans of European and American descent. In some embodiments, SEQ ID NO: 62 is the sequence found predominantly in humans of Asian descent. In some embodiments, a CH3 domain comprises the amino acid sequence GQPREPQVYTLPPSREEMTKNQVSLTCLVKGFYPSDISVEWESNGQPENNYKTTPP MLDSDGSFFLYSKLTVDKSRWQQGNVFSCSVMHEALHNHYTQKSLSLSPGK (SEQ ID NO: 34). In some embodiments, the CH3 domain consists of SEQ ID NO: 34. In some embodiments, SEQ ID NO: 34 is the IgG2 CH3 domain. In some embodiments, a CH3 domain comprises the amino acid sequence GQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK (SEQ ID NO: 35). In some embodiments, the CH3 domain consists of SEQ ID NO: 35. In some embodiments, SEQ ID NO: 35 is the IgG4 CH3 domain. In some embodiments, a CH3 domain comprises the amino acid sequence GQPREPQVYTLPPSREEMTKNQVSLTCLVKGFYPSDIAVEWESSGQPENNYNTTPP MLDSDGSFFLYSKLTVDKSRWQQGNIFSCSVMHEALHNRFTQKSLSLSPGK (SEQ ID NO: 36). In some embodiments, the CH3 domain consists of SEQ ID NO: 36. In some embodiments, SEQ ID NO: 36 is the IgG3 CH3 domain.

In some embodiments, the Fc comprises a mutation. In some embodiments, a CH3 domain comprises a mutation. In some embodiments, the first CH3 domain comprises a first mutation. In some embodiments, the second CH3 domain comprises a second mutation. In some embodiments, a CH2 domain comprises a mutation. In some embodiments, the first CH2 domain comprises a first mutation. In some embodiments, the second CH2 domain comprises a second mutation. In some embodiments, the CH2 and CH3 domains both comprise mutations. In some embodiments, the first CH2 domain and first CH3 domains each comprise a first mutation. In some embodiments, the second CH2 domain and the second CH3 domain each comprise a second mutation. In some embodiments, the mutations inhibit homodimerization of the first polypeptide chain. In some embodiments, the first mutation inhibits homodimerization of the first polypeptide chain. In some embodiments, the mutations inhibit homodimerization of the second polypeptide chain. In some embodiments, the second mutation inhibits homodimerization of the second polypeptide chain. In some, embodiments, the mutations permit heterodimerization. In some embodiments, the mutations permit heterodimerization of the first and second chains. In some embodiments, permitting is promoting. In some embodiments, permitting is enhancing.

Mutations that promote heavy chain heterodimerization and/or inhibit homodimerization are well known in the art. Any such mutations or alterations may be used for constructing the polypeptides of the invention. In some embodiments, a region from an IgG is replaced with a region from an IgA. In some embodiments, a region from a TCRa is inserted into the first CH3 domain and a region from TCRb is inserted in to the second CH3 domain. In some embodiments, the mutation is insertion of a region from a TCR. In some embodiments, the TCR is selected from TCRa and TCRb. In some embodiments, the mutation is insertion of a region from a different Ig. Examples of these mutations can be found in Table 1. In some embodiments, the mutation is selected from a mutation in Table 1. In some embodiments, the first mutation is selected from a group of mutation provided in a row and the second column of Table 1 and the second mutation is the group of mutations provided in that same row of Table 1 in the third column. The mutations in Table 1 are provided with the Kabat numbering for IgG1 unless otherwise stated; corresponding mutations can be made in other IGs and specifically in other IgGs. In some embodiments, the first mutation is T366Y, and the second mutation is Y407T. In some embodiments, the first mutation is S354C and T366W and the second mutation is Y349C, T366S, L368A, and Y407V. In some embodiments, the first mutation is S364H and F405A and the second mutation is Y349T and T392F. In some embodiments, the first mutation is T350V, L351Y, F405A, and Y407V and the second mutation is T350V, T366L, K392L, and T394W. In some embodiments, the first mutation is K392D, and K409D and the second mutation is E356K, and D399K. In some embodiments, the first mutation is D221E, P228E, and L368E and the second mutation is D221R, P228R, and K409R. In some embodiments, the first mutation is K360E, and K409W and the second mutation is Q347R, D399V, and F405T. In some embodiments, the first mutation is K360E, K409W, and Y349C and the second mutation is Q347R, D399V, F405T, and S354C. In some embodiments, the first mutation is F405L and the second mutation is K409R. In some embodiments, the first mutation is K360D, D399M, and Y407A and the second mutation is E345R, Q347R, T366V, and K409V. In some embodiments, the first mutation is Y349S, K370Y, T366M, and K409V and the second mutation is E356G, E357D, S364Q, and Y407A. In some embodiments, the first mutation is T366K, and the second mutation is selected from C351D, Y349E, Y349D, L368E, L368D, Y349E and R355E, Y349E and R355D, Y349D and R355E, and Y349D and R355D. In some embodiments, the first mutation is T366K and C351K and the second mutation is selected from C351D, Y349E, Y349D, L368E, L368D, Y349E and R355E, Y349E and R355D, Y349D and R355E, and Y349D and R355D. In some embodiments, the first mutation is L351D and L368E and the second mutation is L351K and T366K. In some embodiments, the first mutation is L368D and K370S and the second mutation is E357Q and S364K. In some embodiments, the first mutation is T366W, and the second mutation is T366S, L368A and Y407V. In some embodiments, the Ig is IgG2, and the first mutation is C223E, P228E, and L368E and the second mutation is C223R, E225R, P228R, and K409R. In some embodiments, the first mutation is S354C or T366W and the second mutation is Y349C, T366S, L368A, or Y407V. In some embodiments, the first mutation is S364H or F405A and the second mutation is Y349T or T392F. In some embodiments, the first mutation is T350V, L351Y, F405A, or Y407V and the second mutation is T350V, T366L, K392L, or T394W. In some embodiments, the first mutation is K392D, or K409D and the second mutation is E356K, or D399K. In some embodiments, the first mutation is D221E, P228E, or L368E and the second mutation is D221R, P228R, or K409R. In some embodiments, the first mutation is K360E, or K409W and the second mutation is Q347R, D399V, or F405T. In some embodiments, the first mutation is K360E, K409W, or Y349C and the second mutation is Q347R, D399V, F405T, or S354C. In some embodiments, the first mutation is K360D, D399M, or Y407A and the second mutation is E345R, Q347R, T366V, or K409V. In some embodiments, the first mutation is Y349S, K370Y, T366M, or K409V and the second mutation is E356G, E357D, S364Q, or Y407A. In some embodiments, the first mutation is L351D or L368E and the second mutation is L351K or T366K. In some embodiments, the first mutation is L368D or K370S and the second mutation is E357Q or S364K. In some embodiments, the first mutation is T366W, and the second mutation is T366S, L368A or Y407V. In some embodiments, the Ig is IgG2, and the first mutation is C223E, P228E, or L368E and the second mutation is C223R, E225R, P228R, or K409R. In some embodiments, the CH3 domain comprises or consists of GQPREPQVYTLPPSREEMTKNQVSLWCLVKGFYPSDIAVEWESNGQPENNYKTTP PVLDSDGSFFLYSKLTVDKSRWQQGNVFSCSVMHEALHNHYTQKSLSLSPGK (SEQ ID NO: 46). In some embodiments, the CH3 domain comprises or consists of GQPREPQVYTLPPSREEMTKNQVSLSCAVKGFYPSDIAVEWESNGQPENNYKTTPP VLDSDGSFFLVSKLTVDKSRWQQGNVFSCSVMHEALHNHYTQKSLSLSPGK (SEQ ID NO: 47). In some embodiments, the CH3 domain comprises or consists of GQPREPQVYTLPPSREEMTKNQVSLYCLVKGFYPSDIAVEWESNGQPENNYKTTP PVLDSDGSFFLYSKLTVDKSRWQQGNVFSCSVMHEALHNHYTQKSLSLSPGK (SEQ ID NO: 48). In some embodiments, the CH3 domain comprises or consists of

(SEQ ID NO: 49)
GQPREPQVYTLPPSREEMTKNQVSLTCLVKGFYPSDIAVEWESNGQPEN
NYKTTPPVLDSDGSFFLTSKLTVDKSRWQQGNVFSCSVMHEALHNHYTQ
KSLSLSPGK.

TABLE 1
Mutations for enhancing heterodimerization and inhibiting
homodimerization of CH3 domains.

| | | CH3 | |
|---|---|---|---|
| | Strategy | domain Chain 1 | CH3 domain Chain 2 |
| 1 | Knobs-into-holes | T366Y | Y407T |
| | (Y-T) | | |
| 2 | Knobs-into-holes | S354C, T366W | Y349C, T366S, L368A, |
| | (CW-CSAV) | | Y407V |
| 3 | HA-TF | S364H, F405A | Y349T, T394F |
| 4 | ZW1 (VYAV- | T350V, L351Y, | T350V, T366L, K392L, |
| | VLLW) | F405A, Y407V | T394W |
| 5 | CH3 charge pairs | K392D, K409D | E356K, D399K |
| | (DD-KK) | | |
| 6 | Hinge/CH3 charge | D221E, P228E, | D221R, P228R, K409R |
| | (EEE-RRR) | L368E | |
| 7 | EW-RVT | K360E, K409W, | Q347R, D399V, F405T |
| 8 | EW-RVTS-S | K360E, K409W, | Q347R, D399V, F405T, |
| | | Y349C | S354C |
| 9 | (L-R) | F405L | K409R |
| 10 | 7.8.60 (DMA- | K360D, D399M, | E345R, Q347R, T366V, |
| | RRVV) | Y407A | K409V |
| 11 | 20.8.34 (SYMV- | Y349S, K370Y, | E356G, E357D, S364Q, |
| | GDQA) | T366M, K409V | Y407A |
| 12 | Electrostatic | 366K or 366K + | C351D or E or D at 349, |
| | steering effects | C351K | 368, 349, or 349 + 355 |
| 13 | “DEKK” | L351D and L368E | L351K and T366K |
| 14 | XmAb | L368D/K370S | E357Q/S364K |
| 15 | KiH | T366W | T366S/L368A/Y407V |
| 16 | IgG2 hinge/CH3 | IgG2: C223E, | IgG2: C223R, E225R, |
| | charge (EEE- | P228E, L368E | P228R, K409R |
| | RRRR) | | |
| 17 | SEEDbody | IgG/A chimera | IgG/A chimera |
| 18 | BEAT | residues from TCRa | residues from TCRb |
| | | interface | interface |

In some embodiments, the mutation reduces effector function. In some embodiments, effector function comprises ADCC, CDC or both. In some embodiments, reduced effector function comprises reduced cytotoxicity. In some embodiments, reduces is abolishes. In some embodiments, the Fc is from IgG1 or IgG3 and the mutation reduces effector function. In some embodiments, the Fc is from IgG1 and comprises at least one mutation that reduces effector function. Mutations that reduce effector function are well known in the art and any such mutation can be used. Examples of such mutations can be found in Saunders, 2019, “Conceptual approaches to modulating antibody effector functions and circulation half-life” Front Immunol., June 7; 10:1296, herein incorporated by reference in its entirety.

It will be known by a skilled artisan that IgG2 and IgG4 possess greatly reduced effector function and are not generally cytotoxic in nature. Additionally, mutations such as S228P and L235E in IgG4 are known to reduce effector function even more. Further, mutations that reduce the cytotoxicity/effector function of IgG1 and IgG3 are well known in the art. In some embodiments, the IgG comprises at least one mutation. In some embodiments, the mutation is a plurality of mutations. In some embodiments, the mutation decreases cytotoxicity. In some embodiments, the mutation increases stability. In some embodiments, the mutation decreases aggregation. In some embodiments, the plurality of mutations that decreases cytotoxicity comprise the LALA mutations. In some embodiments, the plurality of mutations that decreases cytotoxicity comprise the PG-LALA mutations. In some embodiments, the mutation is mutation of proline 329 of the IgG1 human heavy chain to glycine (P329G). In some embodiments, the P to G mutation is mutation of P109 of SEQ ID NO: 12 to G. In some embodiments, the mutation is mutation of leucine 234 of the IgG1 human heavy chain to alanine (L234A). In some embodiments, the L to A mutation is mutation of L14 of SEQ ID NO: 12 to A. In some embodiments, the mutation is mutation of leucine 235 of the IgG1 human heavy chain to alanine (L235A). In some embodiments, the L to A mutation is mutation of L15 of SEQ ID NO: 12 to A. In some embodiments, the plurality of mutation comprises P109G, L14A and L15A of SEQ ID NO: 12. In some embodiments, the plurality of mutation comprises L14A and L15A of SEQ ID NO: 12. In some embodiments, the plurality of mutation comprises P329G, L234A and L235A of the IgG1 human heavy chain. In some embodiments, the plurality of mutation comprises L234A and L235A of the IgG1 human heavy chain. It will be understood by a skilled artisan that parallel mutation can also be performed in the IgG3 heavy chain or the heavy chains of non-human IgG1s. In some embodiments, the plurality of mutations that decreases cytotoxicity comprise the YTE mutations. In some embodiments, the mutation is mutation of methionine 252 of the IgG1 human heavy chain to tyrosine (M252Y). In some embodiments, the M to Y mutation is mutation of M32 of SEQ ID NO: 12 to Y. In some embodiments, the mutation is mutation of serine 254 of the IgG1 human heavy chain to threonine (S254T). In some embodiments, the S to T mutation is mutation of S34 of SEQ ID NO: 12 to T. In some embodiments, the mutation is mutation of threonine 256 of the IgG1 human heavy chain to glutamic acid (T256E). In some embodiments, the T to E mutation is mutation of T36 of SEQ ID NO: 12 to E. In some embodiments, the plurality of mutation comprises M32Y, S34T and T36E of SEQ ID NO: 12. In some embodiments, the plurality of mutation comprises M252Y, S254T and T256E of the IgG1 human heavy chain. In some embodiments, the mutation is mutation of asparagine 297 of the IgG1 human heavy chain (N297). In some embodiments, the asparagine is mutated to alanine (N297A). In some embodiments, the asparagine is mutated to glutamine (N297Q). In some embodiments, the asparagine is N77 of SEQ ID NO: 12 (N77A or N77Q). It will be understood by a skilled artisan that parallel mutation can also be performed in the IgG3 heavy chain or the heavy chains of non-human IgG1s. It will be understood that the number given herein is in reference to a full-length IgG including the variable domains. The numbers can be shifted to correspond to the positions of these amino acids within just the Fc portion of the IgG.

### Third and Fourth Chains

In some embodiments, the protein complex further comprises a third polypeptide chain. In some embodiments, the third polypeptide chain comprises a third fragment of a protein target of myasthenia gravis autoantibodies. In some embodiments, the third fragment is different than the first fragment. In some embodiments, the third fragment is different than the second fragment. In some embodiments, the third fragment is the same as the first fragment. In some embodiments, the first fragment is the same as the second fragment. In some embodiments, the third fragment is the same as the first and second fragments. In some embodiments, the same as is the same sequence. In some embodiments, different is a different sequence.

In some embodiments, the third polypeptide further comprises a third dimerization domain. In some embodiments, the first polypeptide further comprises a fourth dimerization domain. In some embodiments, the third and fourth dimerization domains are capable of dimerizing to each other. In some embodiments, the third and fourth dimerization domains are configured to dimerizing to each other. In some embodiments, the third dimerization domain is not configured to dimerize to the first dimerization domain. In some embodiments, the third dimerization domain is not configured to dimerize to the second dimerization domain. In some embodiments, the fourth dimerization domain is not configured to dimerize to the first dimerization domain. In some embodiments, the fourth dimerization domain is not configured to dimerize to the second dimerization domain. In some embodiments, configured to dimerize is capable of dimerizing. In some embodiments, the third and fourth dimerization domains are different than the first and second dimerization domains. In some embodiments, the first and second dimerization domains are hinge domains and the third and fourth dimerization domains are CH1/CL domains. In some embodiments, the first and second dimerization domains are CH1/CL domains and the third and fourth dimerization domains are hinge domains.

In some embodiments, the protein complex further comprises a fourth polypeptide chain. In some embodiments, the fourth polypeptide chain comprises a fourth fragment of a protein target of myasthenia gravis autoantibodies. In some embodiments, the fourth fragment is different than the first fragment. In some embodiments, the fourth fragment is different than the second fragment. In some embodiments, the fourth fragment is different than the third fragment. In some embodiments, the fourth fragment is the same as the first fragment. In some embodiments, the fourth fragment is the same as the second fragment. In some embodiments, the fourth fragment is the same as the third fragment. In some embodiments, the fourth fragment is the same as the first, second and third fragments. In some embodiments, the first, second, and third fragments are all the same. In some embodiments, the first, second, third and fourth fragments are all different. In some embodiments, the same as is the same sequence. In some embodiments, different is a different sequence. In some embodiments, different is from a different protein. In some embodiments, different is from the same protein but comprising a different sequence. In some embodiments, different is from the same protein but from a different region of the protein. In some embodiments, at least two of the first, second, third and fourth proteins are part of a single protein complex. In some embodiments, the protein complex is a complex in mammals. In some embodiments, the protein complex is a complex in humans.

In some embodiments, the fourth polypeptide further comprises a fifth dimerization domain. In some embodiments, the second polypeptide further comprises a sixth dimerization domain. In some embodiments, the fifth and sixth dimerization domains are capable of dimerizing to each other. In some embodiments, the fifth and sixth dimerization domains are configured to dimerizing to each other. In some embodiments, the fifth dimerization domain is not configured to dimerize to the first dimerization domain. In some embodiments, the fifth dimerization domain is not configured to dimerize to the second dimerization domain. In some embodiments, the fifth dimerization domain is not configured to dimerize to the third dimerization domain. In some embodiments, the fifth dimerization domain is not configured to dimerize to the fourth dimerization domain. In some embodiments, the sixth dimerization domain is not configured to dimerize to the first dimerization domain. In some embodiments, the sixth dimerization domain is not configured to dimerize to the second dimerization domain. In some embodiments, the sixth dimerization domain is not configured to dimerize to the third dimerization domain. In some embodiments, the sixth dimerization domain is not configured to dimerize to the fourth dimerization domain. In some embodiments, the fifth and sixth dimerization domains are different than the first and second dimerization domains. In some embodiments, the fifth and sixth dimerization domains are different than the third and fourth dimerization domains. In some embodiments, the first and second dimerization domains are hinge domains, the third and fourth dimerization domains are CH1/CL domains and the fifth and sixth dimerization domains are CH1/CL domains. In some embodiments, the first and second dimerization domains are CH1/CL domains, the third and fourth dimerization domains are hinge domains and the fifth and sixth dimerization domains are hinge domains. In some embodiments, the first polypeptide and second polypeptide do not both comprise a CH1 domain. In some embodiments, first polypeptide and second polypeptide both comprise a CH1 domain. first polypeptide and second polypeptide both comprise a CL domain. In some embodiments, first polypeptide and second polypeptide do not both comprise a CL domain. In some embodiments, the first polypeptide comprises a CH1 domain, and the second polypeptide comprises a CL domain. In some embodiments, the third polypeptide comprises a CL domain and the fourth polypeptide comprise a CH1 domain. In some embodiments, the first polypeptide comprises a CL domain and the second polypeptide comprises a CH1 domain. In some embodiments, the third polypeptide comprises a CH1 domain, and the fourth polypeptide comprise a CL domain.

In some embodiments, the third and fourth dimerization domains comprises mutations that permit dimerization of the third and fourth dimerization domains and inhibit dimerization of the third dimerization domain to the fifth, sixth or both dimerization domains. In some embodiments, the third and fourth dimerization domains comprises mutations that permit dimerization of the third and fourth dimerization domains and inhibit dimerization of the fourth dimerization domain to the fifth, sixth or both dimerization domains. In some embodiments, the fifth and sixth dimerization domains comprises mutations that permit dimerization of the fifth and sixth dimerization domains and inhibit dimerization of the fifth dimerization domain to the third, fourth or both dimerization domains. In some embodiments, the fifth and sixth dimerization domains comprises mutations that permit dimerization of the fifth and sixth dimerization domains and inhibit dimerization of the sixth dimerization domain to the third, sixth or both dimerization domains.

### Alternative Configurations

In some embodiments, the composition comprises a polypeptide chain comprising the fragment of a first protein target of myasthenia gravis autoantibodies or an analog or derivative thereof and the fragment of a second protein target of myasthenia gravis autoantibodies or an analog or derivative thereof. In some embodiments, the polypeptide chain is a single polypeptide chain. In some embodiments, the single chain comprises the fragment of the first protein and the fragment of the second protein. In some embodiments, the polypeptide chain further comprises a fragment of a third protein target of myasthenia gravis autoantibodies or an analog or derivative thereof. In some embodiments, the polypeptide chain further comprises a fragment of a fourth protein target of myasthenia gravis autoantibodies or an analog or derivative thereof. In some embodiments, the polypeptide chain further comprises an Fc region.

In some embodiments, the fragment of the first protein target of myasthenia gravis autoantibodies or an analog or derivative thereof is separated from the fragment of the second protein target of myasthenia gravis autoantibodies or an analog or derivative thereof by a linker. In some embodiments, the fragment of the third protein target of myasthenia gravis autoantibodies or an analog or derivative thereof is separated from the fragment of the first or the second protein target of myasthenia gravis autoantibodies or an analog or derivative thereof by a linker. In some embodiments, the fragment of the fourth protein target of myasthenia gravis autoantibodies or an analog or derivative thereof is separated from the fragment of the first, the second or the third protein target of myasthenia gravis autoantibodies or an analog or derivative thereof by a linker. In some embodiments, a fragment is separated from the Fc region by a linker.

In some embodiments, the fragment and the dimerization domain are separated by a linker. In some embodiments, the dimerization domain and the Fc region are separated by a linker. In some embodiments, the fragment and the Fc region are separated by a linker. In some embodiments, the linker is an amino acid linker. In some embodiments, the linker is a chemical linker. In some embodiments, the linker is a peptide linker. In some embodiments, the linker is a bond. In some embodiments, the bond is a peptide bond. In some embodiments, the bond is an amino acid bond. In some embodiments, the linker is a flexible linker. Linkers are well known in the art and any linker may be used. In some embodiments, a linker is a chemical linker.

In some embodiments, conjugated is linked. In some embodiments, conjugation is via a bond. In some embodiments, the conjugate is directly conjugated. In some embodiments, the conjugate is conjugated via a linker.

In some embodiments, the linker is of a sufficient length to inhibit steric hindrance between different sections of the chain. In some embodiments, the linker is of a sufficient length to inhibit steric hindrance between different sections of the conjugate. In some embodiments, the linker is of a sufficient length to allow binding of an antibody to the fragment without steric hindrance from another section of the chain. In some embodiments, the linker is of a sufficient length to allow binding of an antibody to the fragment without steric hindrance from another section of the conjugate. In some embodiments, the linker is of a sufficient length to allow binding of a cell to the fragment without steric hindrance from another section of the chain. In some embodiments, the linker is of a sufficient length to allow binding of a cell to the fragment without steric hindrance from another section of the conjugate. In some embodiments, the linker is at least 1, 2, 3, 4, 5, 6, 7, 8, 9, 10, 11, 12, 13, 14 or 15 amino acids in length. Each possibility represents a separate embodiment of the invention. In some embodiments, the linker is at least 1 amino acid in length. In some embodiments, the linker is at least 5 amino acids in length. In some embodiments, the linker is at least 10 amino acids in length. In some embodiments, the linker is at least 15 amino acids in length. In some embodiments, the linker is at most 10, 15, 20, 25, 30, 35, 40, 45, 50, 60, 70, 80, 90 or 100 amino acids in length. Each possibility represents a separate embodiment of the invention. In some embodiments, the linker is at most 10 amino acids in length. In some embodiments, the linker is at most 20 amino acids in length. In some embodiments, the linker is at most 50 amino acids in length. In some embodiments, the linker is at most 100 amino acids in length.

In some embodiments, the linker is a flexible linker. In some embodiments, the linker is a GS linker. In some embodiments, the linker is a glycine-serine containing linker. In some embodiments, the linker consists of glycine and serine residues. In some embodiments, the linker comprises GGGS (SEQ ID NO: 38). In some embodiments, the linker comprises GGGGS (SEQ ID NO: 136). In some embodiments, the linker consists of SEQ ID NO: 38. In some embodiments, the linker consists of SEQ ID NO: 136. In some embodiments, the linker comprises (GGGS)n wherein n is an integer. In some embodiments, the linker comprises (GGGGS)n wherein n is an integer. In some embodiments, the linker consists of (GGGS)n wherein n is an integer. In some embodiments, the linker consists of (GGGGS)n wherein n is an integer. In some embodiments, the linker comprises GSAGSAAGSGEF (SEQ ID NO: 45). In some embodiments, the linker comprises or consists of (GGGS)nGS wherein n is an integer. In some embodiments, n is selected from 1, 2, 3, 4, 5 and 6. Each possibility represents a separate embodiment of the invention. In some embodiments, n is 6. In some embodiments, the linker is a rigid linker. In some embodiments, the rigid linker comprises EAAAK (SEQ ID NO: 137). In some embodiments, the rigid linker consists of SEQ ID NO: 137. In some embodiments, the rigid linker comprises (EAAAK)n where n is an integer. In some embodiments, the rigid linker consists of (EAAAK)n where n is an integer. In some embodiments, the rigid linker comprises (EAAAK)nGS where n is an integer. In some embodiments, the rigid linker consists of (EAAAK)nGS where n is an integer. In some embodiments, the rigid linker comprises (EAAAK)nGGS where n is an integer. In some embodiments, the rigid linker consists of (EAAAK)nGGS where n is an integer. In some embodiments, n is selected from 1, 2, 3, 4, 5, 6, 7, 8, 9 and 10. Each possibility represents a separate embodiment of the invention. In some embodiments, n is 2. In some embodiments, n is 3. In some embodiments, n is 4. In some embodiments, n is 5.

In some embodiments, the dimerization domain is C-terminal to the fragment. In some embodiments, the fragment is C-terminal to the dimerization domain. In some embodiments, the Fc region is C-terminal to the fragment. In some embodiments, the fragment is C-terminal to the Fc region. In some embodiments, the dimerization domain is C-terminal to the Fc region. In some embodiments, the Fc region is C-terminal to the dimerization domain. In some embodiments, the dimerization domain is N-terminal to the fragment. In some embodiments, the fragment is N-terminal to the dimerization domain. In some embodiments, the Fc region is N-terminal to the fragment. In some embodiments, the fragment is N-terminal to the Fc region. In some embodiments, the dimerization domain is N-terminal to the Fc region. In some embodiments, the Fc region is N-terminal to the dimerization domain.

In some embodiments, the epitope spans at least two fragments. In some embodiments, the epitope spans the first and second fragments. In some embodiments, the epitope spans the first and third fragments. In some embodiments, the epitope spans the first and fourth fragments. In some embodiments, the epitope spans the second and third fragments. In some embodiments, the epitope spans the second and fourth fragments. In some embodiments, the epitope spans the third and fourth fragments. In some embodiments, the epitope spans two proteins. In some embodiments, the epitope spans two proteins in a protein complex. In some embodiments, the epitope spans three fragments. In some embodiments, the epitope spans three proteins. In some embodiments, the epitope spans four fragments. In some embodiments, the epitope spans four proteins. In some embodiments, the epitope is a complex epitope. In some embodiments, the epitope is a B cell receptor (BCR)-specific epitope.

In some embodiments, all three fragments are from AChRa. In some embodiments, all three fragments are from AChRb. In some embodiments, all three fragments are from AChRg. In some embodiments, all three fragments are from AChRd. In some embodiments, all three fragments are from AChRe. In some embodiments, the three fragments are selected from AChRa, AChRb, AChRg, AChRd and AChRe. In some embodiments, the three fragments comprise two different proteins from AChRa, AChRb, AChRg, AChRd and AChRe. In some embodiments, the three fragments comprise three different proteins from AChRa, AChRb, AChRg, AChRd and AChRe.

In some embodiments, all four fragments are from AChRa. In some embodiments, all four fragments are from AChRb. In some embodiments, all four fragments are from AChRg. In some embodiments, all four fragments are from AChRd. In some embodiments, all four fragments are from AChRe. In some embodiments, the four fragments are selected from AChRa, AChRb, AChRg, AChRd and AChRe. In some embodiments, the four fragments comprise two different proteins from AChRa, AChRb, AChRg, AChRd and AChRe. In some embodiments, four fragments comprise three different proteins from AChRa, AChRb, AChRg, AChRd and AChRe. In some embodiments, the four fragments comprise four different proteins from AChRa, AChRb, AChRg, AChRd and AChRe.

In some embodiments, the first polypeptide comprises a fragment linked to ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKA KGQPREPQVYTLPPSREEMTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 50). In some embodiments, the second polypeptide comprises a fragment linked to SEQ ID NO: 50. In some embodiments, the first and second polypeptides both comprises a fragment linked to SEQ ID NO: 50.

In some embodiments, the first polypeptide comprises a fragment linked to ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKA KGQPREPQVYTLPPSREEMTKNQVSLWCLVKGFYPSDIAVEWESNGQPENNYKTT PPVLDSDGSFFLYSKLTVDKSRWQQGNVFSCSVMHEALHNHYTQKSLSLSPGK (SEQ ID NO: 51). In some embodiments, the second polypeptide comprises a fragment linked to SEQ ID NO: 51. In some embodiments, the first and second polypeptides both comprises a fragment linked to SEQ ID NO: 51.

In some embodiments, the first polypeptide comprises a fragment linked to ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKA KGQPREPQVYTLPPSREEMTKNQVSLSCAVKGFYPSDIAVEWESNGQPENNYKTT PPVLDSDGSFFLVSKLTVDKSRWQQGNVFSCSVMHEALHNHYTQKSLSLSPGK (SEQ ID NO: 52). In some embodiments, the second polypeptide comprises a fragment linked to SEQ ID NO: 52. In some embodiments, the first and second polypeptides both comprises a fragment linked to SEQ ID NO: 52.

In some embodiments, the first polypeptide comprises a fragment linked to ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKA KGQPREPQVYTLPPSREEMTKNQVSLYCLVKGFYPSDIAVEWESNGQPENNYKTT PPVLDSDGSFFLYSKLTVDKSRWQQGNVFSCSVMHEALHNHYTQKSLSLSPGK (SEQ ID NO: 53). In some embodiments, the second polypeptide comprises a fragment linked to SEQ ID NO: 53. In some embodiments, the first and second polypeptides both comprises a fragment linked to SEQ ID NO: 53.

In some embodiments, the first polypeptide comprises a fragment linked to ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKA KGQPREPQVYTLPPSREEMTKNQVSLTCLVKGFYPSDIAVEWESNGQPENNYKTT PPVLDSDGSFFLTSKLTVDKSRWQQGNVFSCSVMHEALHNHYTQKSLSLSPGK (SEQ ID NO: 54). In some embodiments, the second polypeptide comprises a fragment linked to SEQ ID NO: 54. In some embodiments, the first and second polypeptides both comprises a fragment linked to SEQ ID NO: 54.

In some embodiments, the first polypeptide comprises a fragment linked to AAPSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGECDKTHTC PPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYK TTPPVLDSDGSFFLYSKLTVDKSRWQQGNVFSCSVMHEALHNHYTQKSLSLSPGK (SEQ ID NO: 55). In some embodiments, the second polypeptide comprises a fragment linked to SEQ ID NO: 55. In some embodiments, the first and second polypeptides both comprises a fragment linked to SEQ ID NO: 55.

In some embodiments, the first polypeptide comprises a fragment linked to GQPKANPTVTLFPPSSEELQANKATLVCLISDFYPGAVTVAWKADGSPVKAGVET TKPSKQSNNKYAASSYLSLTPEQWKSHRSYSCQVTHEGSTVEKTVAPTECSDKTH TCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEK TISKAKGQPREPQVYTLPPSREEMTKNQVSLTCLVKGFYPSDIAVEWESNGQPENN YKTTPPVLDSDGSFFLYSKLTVDKSRWQQGNVFSCSVMHEALHNHYTQKSLSLSP GK (SEQ ID NO: 56). In some embodiments, the second polypeptide comprises a fragment linked to SEQ ID NO: 56. In some embodiments, the first and second polypeptides both comprises a fragment linked to SEQ ID NO: 56.

In some embodiments, the first polypeptide comprises a fragment linked to AAPSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGECDKTHTC PPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTIS KAKGQPREPQVYTLPPSREEMTKNQVSLSCAVKGFYPSDIAVEWESNGQPENNYK TTPPVLDSDGSFFLVSKLTVDKSRWQQGNVFSCSVMHEALHNHYTQKSLSLSPGK (SEQ ID NO: 57). In some embodiments, the second polypeptide comprises a fragment linked to SEQ ID NO: 57. In some embodiments, the first and second polypeptides both comprises a fragment linked to SEQ ID NO: 57.

In some embodiments, the first polypeptide comprises a fragment linked to AAPSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGECDKTHTC PPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTIS KAKGQPREPQVYTLPPSREEMTKNQVSLWCLVKGFYPSDIAVEWESNGQPENNY KTTPPVLDSDGSFFLYSKLTVDKSRWQQGNVFSCSVMHEALHNHYTQKSLSLSPG K (SEQ ID NO: 58). In some embodiments, the second polypeptide comprises a fragment linked to SEQ ID NO: 58. In some embodiments, the first and second polypeptides both comprises a fragment linked to SEQ ID NO: 58.

In some embodiments, the first polypeptide comprises a fragment linked to GQPKANPTVTLFPPSSEELQANKATLVCLISDFYPGAVTVAWKADGSPVKAGVET TKPSKQSNNKYAASSYLSLTPEQWKSHRSYSCQVTHEGSTVEKTVAPTECSDKTH TCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEK TISKAKGQPREPQVYTLPPSREEMTKNQVSLSCAVKGFYPSDIAVEWESNGQPENN YKTTPPVLDSDGSFFLVSKLTVDKSRWQQGNVFSCSVMHEALHNHYTQKSLSLSP GK (SEQ ID NO: 59). In some embodiments, the second polypeptide comprises a fragment linked to SEQ ID NO: 59. In some embodiments, the first and second polypeptides both comprises a fragment linked to SEQ ID NO: 59.

In some embodiments, the first polypeptide comprises a fragment linked to GQPKANPTVTLFPPSSEELQANKATLVCLISDFYPGAVTVAWKADGSPVKAGVET TKPSKQSNNKYAASSYLSLTPEQWKSHRSYSCQVTHEGSTVEKTVAPTECSDKTH TCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEK TISKAKGQPREPQVYTLPPSREEMTKNQVSLWCLVKGFYPSDIAVEWESNGQPEN NYKTTPPVLDSDGSFFLYSKLTVDKSRWQQGNVFSCSVMHEALHNHYTQKSLSLS PGK (SEQ ID NO: 60). In some embodiments, the second polypeptide comprises a fragment linked to SEQ ID NO: 60. In some embodiments, the first and second polypeptides both comprises a fragment linked to SEQ ID NO: 60.

In some embodiments, the third polypeptide comprises a fragment linked to SEQ ID NO: 23. In some embodiments, the third polypeptide comprises a fragment linked to SEQ ID NO: 24. In some embodiments, the third polypeptide comprises a fragment linked to SEQ ID NO: 25. In some embodiments, the third polypeptide comprises a fragment linked to SEQ ID NO: 26. In some embodiments, the third polypeptide comprises a fragment linked to SEQ ID NO: 27. In some embodiments, the third polypeptide comprises a fragment linked to SEQ ID NO: 28. In some embodiments, the fourth polypeptide comprises a fragment linked to SEQ ID NO: 23. In some embodiments, the fourth polypeptide comprises a fragment linked to SEQ ID NO: 24. In some embodiments, the fourth polypeptide comprises a fragment linked to SEQ ID NO: 25. In some embodiments, the fourth polypeptide comprises a fragment linked to SEQ ID NO: 26. In some embodiments, the fourth polypeptide comprises a fragment linked to SEQ ID NO: 27. In some embodiments, the fourth polypeptide comprises a fragment linked to SEQ ID NO: 28.

In some embodiments, a polypeptide chain comprises a fragment of AChRa comprising a mutation to increase solubility and linked via a linker to a light chain CL kappa domain. In some embodiments, the polypeptide comprises or consists of the amino acid sequence

(SEQ ID NO: 64)
SEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADG
DFAIVKFTKVLLQYTGHITWTPPAIFKSYCDVSGVDTESGATNCSMKLG
TWTYDGSVVAINPESDQPDLSNFMESGEWVIKESRGWKHSVTYSCCPDT
PYLDITYHFVMQRLPGGGGSGGGGSGGGGSAAPSVFIFPPSDEQLKSGT
ASVVCLLNNFYPREAKVQWKVDNALQSGNSQESVTEQDSKDSTYSLSST
LTLSKADYEKHKVYACEVTHQGLSSPVTKSFNRGEC.

In some embodiments, a polypeptide chain comprises a fragment of AChRa comprising a mutation to increase solubility and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 92. In some embodiments, the polypeptide comprises or consists of the amino acid sequence of SEQ ID NO: 95. In some embodiments, the polypeptide comprises or consists of the amino acid sequence of SEQ ID NO: 97. In some embodiments, the polypeptide comprises or consists of the amino acid sequence of SEQ ID NO: 98. In some embodiments, the polypeptide comprises or consists of the amino acid sequence of SEQ ID NO: 99. It will be understood that the above described CH2 and CH3 domains and all other CH2/CH3 domains unless explicitly stated otherwise are from IgG1.

In some embodiments, a polypeptide chain comprises a fragment of AChRb comprising a mutation to increase solubility and linked via a linker to a heavy chain comprising CH1, CH2 and CH3 domains. In some embodiments, the polypeptide comprises or consists of the amino acid sequence

(SEQ ID NO: 65)
SEAEGRLREKLFSGYDSSVRPAREVGDRVRVSVGLILAQLISLNEKDEE
MSTKVYLDLEWTDYRLSWDPAEHDGIDSLRITAESVWLPDVVLLNNNDG
NFDVALDISVVVSSDGSVRWQPPGIYRSSCDVSGVDTESGATNCTMVFS
SYSYDSSEVSLQTGLGPDGQGHQEIHIHEGTFIENGQWEIIHKPSRLIQ
PPGDPRGGREGQRQEVIFYLIIRRKPGGGGSGGGGSGGGGSASTKGPSV
FPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQ
GNVFSCSVMHEALHNHYTQKSLSLSPGK.

In some embodiments, a polypeptide chain comprises a fragment of AChRb comprising a mutation to increase solubility and linked via a linker to a heavy chain comprising CH1, hinge, CH2 and CH3 domains. In some embodiments, the polypeptide comprises or consists of the amino acid sequence SEQ ID NO: 65.

In some embodiments, the protein complex comprises two polypeptides each comprising SEQ ID NO: 65. In some embodiments, the protein complex comprises two polypeptides each consisting of SEQ ID NO: 65. In some embodiments, the protein complex comprises a first polypeptide chain comprising or consisting of SEQ ID NO: 65 and a second polypeptide chain comprising or consisting of SEQ ID NO: 64. In some embodiments, the protein complex further comprises a third polypeptide chain comprising or consisting of SEQ ID NO: 65. In some embodiments, the protein complex further comprises a fourth polypeptide chain comprising of consisting of SEQ ID NO: 64.

In some embodiments, a polypeptide chain comprises a fragment of AChRb comprising a mutation to increase solubility and linked via a linker to a heavy chain comprising CH1, hinge, CH2 and CH3 domains, wherein the CH3 domain comprises a mutation that inhibits homodimerization of the polypeptide chain. In some embodiments, the mutation that inhibits homodimerization is T366W. In some embodiments, the polypeptide comprises or consists of the amino acid sequence

(SEQ ID NO: 66)
SEAEGRLREKLFSGYDSSVRPAREVGDRVRVSVGLILAQLISLNEKDEE
MSTKVYLDLEWTDYRLSWDPAEHDGIDSLRITAESVWLPDVVLLNNNDG
NFDVALDISVVVSSDGSVRWQPPGIYRSSCDVSGVDTESGATNCTMVFS
SYSYDSSEVSLQTGLGPDGQGHQEIHIHEGTFIENGQWEIIHKPSRLIQ
PPGDPRGGREGQRQEVIFYLIIRRKPGGGGSGGGGSGGGGSASTKGPSV
FPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKC
KVSNKALPAPIEKTISKAKGQPREPQVYTLPPSREEMTKNQVSLWCLVK
GFYPSDIAVEWESNGQPENNYKTTPPVLDSDGSFFLYSKLTVDKSRWQQ
GNVFSCSVMHEALHNHYTQKSLSLSPGK.

In some embodiments, a polypeptide chain comprises a fragment of AChRg comprising a mutation to increase solubility and linked via a linker to a heavy chain comprising CH1, hinge, CH2 and CH3 domains, wherein the CH3 domain comprises a mutation that inhibits homodimerization of the polypeptide chain. In some embodiments, the mutation that inhibits homodimerization is T366S, L368A and Y407V. In some embodiments, the polypeptide comprises or consists of the amino acid sequence

(SEQ ID NO: 67)
RNQEERLLADLMQNYDPNLRPAERDSDVVNVSLKLTLTNLISLNEREEA
LTTNVWIEMQWCDYRLRWDPRDYEGLWVLRVPSTMVWRPDIVLENNVDG
VFEVALYCNVLVSPDGCIYWLPPAIFRSACDVSGVDTESGATNCSLIFQ
SQTYSTNEIDLQLSQEDGQTIEWIFIDPEAFTENGEWAIQHRPAKMLLD
PAAPAQEAGHQKVVFYLLIQRKPGGGGSGGGGSGGGGSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLSCAVKGFY
PSDIAVEWESNGQPENNYKTTPPVLDSDGSFFLVSKLTVDKSRWQQGNV
FSCSVMHEALHNHYTQKSLSLSPGK.

In some embodiments, a polypeptide chain comprises a fragment of AChRg comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 93. In some embodiments, the polypeptide comprises or consists of the amino acid sequence of SEQ ID NO: 100.

In some embodiments, a polypeptide chain comprises a fragment of AChRd comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 98. In some embodiments, the polypeptide comprises or consists of the amino acid sequence of SEQ ID NO: 102. In some embodiments, the polypeptide comprises or consists of the amino acid sequence of SEQ ID NO: 103. In some embodiments, the polypeptide comprises or consists of the amino acid sequence of SEQ ID NO: 106. In some embodiments, the polypeptide comprises or consists of the amino acid sequence of SEQ ID NO: 107. In some embodiments, the polypeptide comprises or consists of the amino acid sequence of SEQ ID NO: 130. In some embodiments, the composition comprises a first chain comprising or consisting of SEQ ID NO: 92 and a second chain comprising or consisting of SEQ ID NO: 93. In some embodiments, the composition comprises a first chain comprising or consisting of SEQ ID NO: 92 and a second chain comprising or consisting of SEQ ID NO: 102. In some embodiments, the composition comprises a first chain comprising or consisting of SEQ ID NO: 103 and a second chain comprising or consisting of SEQ ID NO: 102. It will be understood by a skilled artisan that in this embodiment the first polypeptide comprises the T366W mutation and the second polypeptide comprises the T366S/L368A/Y407V mutations, but that the mutations could be switched to the opposite chains and the molecule would still be operable.

In some embodiments, a polypeptide chain comprises a fragment of AChRa comprising a mutation to increase solubility linked via a GS linker to AChRg comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 94. In some embodiments, the polypeptide comprises or consists of the amino acid sequence of SEQ ID NO: 104.

In some embodiments, a polypeptide chain comprises a fragment of AChRa comprising a mutation to increase solubility linked via a GS linker to AChRg comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 95. In some embodiments, the polypeptide comprises or consists of the amino acid sequence of SEQ ID NO: 105.

In some embodiments, a polypeptide chain comprises a fragment of AChRa comprising a mutation to increase solubility linked via a GS linker to AChRd comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 96. In some embodiments, the composition comprises a first chain comprising or consisting of SEQ ID NO: 95 and a second chain comprising or consisting of SEQ ID NO: 96. In some embodiments, the polypeptide comprises or consists of the amino acid sequence of SEQ ID NO: 106. In some embodiments, the polypeptide comprises or consists of the amino acid sequence of SEQ ID NO: 107. In some embodiments, the composition comprises a first chain comprising or consisting of SEQ ID NO: 105 and a second chain comprising or consisting of SEQ ID NO: 106. In some embodiments, the composition comprises a first chain comprising or consisting of SEQ ID NO: 105 and a second chain comprising or consisting of SEQ ID NO: 107. It will be understood by a skilled artisan that in this embodiment the first polypeptide comprises the T366W mutation and the second polypeptide comprises the T366S/L368A/Y407V mutations, but that the mutations could be switched to the opposite chains and the molecule would still be operable.

In some embodiments, a polypeptide chain comprises a fragment of AChRa comprising a mutation to increase solubility and a mutation to decrease aggregation linked via a GS linker to AChRg comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 97. In some embodiments, a polypeptide chain comprises a fragment of AChRa comprising a mutation to increase solubility and a mutation to decrease aggregation linked via a GS linker to AChRg comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 114.

In some embodiments, a polypeptide chain comprises a fragment of AChRd comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 99. In some embodiments, the composition comprises a first chain comprising or consisting of SEQ ID NO: 97 and a second chain comprising or consisting of SEQ ID NO: 98. It will be understood by a skilled artisan that in this embodiment the first polypeptide comprises the T366W mutation and the second polypeptide comprises the T366S/L368A/Y407V mutations, but that the mutations could be switched to the opposite chains and the molecule would still be operable.

In some embodiments, a polypeptide chain comprises a fragment of AChRa comprising a mutation to increase solubility and a mutation to decrease aggregation linked via a GS linker to AChRd comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 99. In some embodiments, the polypeptide comprises or consists of the amino acid sequence of SEQ ID NO: 106. In some embodiments, the polypeptide comprises or consists of the amino acid sequence of SEQ ID NO: 107.

In some embodiments, a polypeptide chain comprises a fragment of AChRg comprising a mutation to increase solubility and at least one mutation to decrease aggregation and linked via a linker to a heavy chain comprising CH2 and CH3 domains, wherein the CH3 domain comprises a mutation that inhibits homodimerization of the polypeptide chain. In some embodiments, the polypeptide comprises or consists of the amino acid sequence of SEQ ID NO: 100. In some embodiments, the composition comprises a first chain comprising or consisting of SEQ ID NO: 99 and a second chain comprising or consisting of SEQ ID NO: 100. In some embodiments, the composition comprises a first chain comprising or consisting of SEQ ID NO: 92 and a second chain comprising or consisting of SEQ ID NO: 102. In some embodiments, the composition comprises a first chain comprising or consisting of SEQ ID NO: 103 and a second chain comprising or consisting of SEQ ID NO: 100. In some embodiments, the composition comprises a first chain comprising or consisting of SEQ ID NO: 105 and a second chain comprising or consisting of SEQ ID NO: 130. In some embodiments, the composition comprises a first chain comprising or consisting of SEQ ID NO: 105 and a second chain comprising or consisting of SEQ ID NO: 106. In some embodiments, the composition comprises a first chain comprising or consisting of SEQ ID NO: 105 and a second chain comprising or consisting of SEQ ID NO: 107. It will be understood by a skilled artisan that in this embodiment the first polypeptide comprises the T366W mutation and the second polypeptide comprises the T366S/L368A/Y407V mutations, but that the mutations could be switched to the opposite chains and the molecule would still be operable.

In some embodiments, a polypeptide chain comprises a fragment of AChRg comprising a mutation to increase solubility and at least one mutation to decrease aggregation linked via a GS linker to AChRa comprising a mutation to increase solubility linked via a GS linker to AChRd comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 108. In some embodiments, a polypeptide chain comprises a fragment of AChRg comprising a mutation to increase solubility and at least one mutation to decrease aggregation linked via a GS linker to AChRa comprising a mutation to increase solubility linked via a GS linker to AChRg comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 109. In some embodiments, the polypeptide comprises or consists of the amino acid sequence of SEQ ID NO: 116.

In some embodiments, a polypeptide chain comprises a fragment of AChRg comprising a mutation to increase solubility and at least one mutation to decrease aggregation linked via a GS linker to AChRa comprising a mutation to increase solubility and a mutation to decrease aggregation linked via a GS linker to AChRg comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 115.

In some embodiments, a polypeptide chain comprises a fragment of AChRa comprising a mutation to increase solubility and a mutation to decrease solubility linked via a GS linker to AChRg comprising a mutation to increase solubility and at least one mutation to decrease aggregation and linked via a linker to a heavy chain comprising CH2 and CH3 domains. In some embodiments, the polypeptide comprises or consists of the amino acid sequence of SEQ ID NO: 110. In some embodiments, the polypeptide comprises or consists of the amino acid sequence of SEQ ID NO: 111. It will be understood by a skilled artisan that in cases of tandem subunits separated by a linker the order of the subunits can be as recited hereinabove or can be switched.

In some embodiments, a polypeptide chain comprises a fragment of AChRa comprising a mutation to increase solubility linked via a GS linker to AChRg comprising a mutation to increase solubility and at least one mutation to decrease aggregation and linked via a linker to a heavy chain comprising CH2 and CH3 domains from IgG4. In some embodiments, the polypeptide comprises or consists of the amino acid sequence of SEQ ID NO: 112. In some embodiments, within any one of SEQ ID NO: 92-111, 114-116 and 124-126 the CH2 and CH3 domains from IgG1 are replaced with CH2 and CH3 domains from IgG4. It will be understood that any mutations present to decrease homodimerization will be conserved and also present in the IgG4 CH3.

In some embodiments, a polypeptide chain comprises a fragment of AChRa comprising a mutation to increase solubility linked via a GS linker to AChRg comprising a mutation to increase solubility and at least one mutation to decrease aggregation and linked via a linker to a heavy chain comprising CH2 and CH3 comprising a mutation that decreases effector function. In some embodiments, the polypeptide comprises or consists of the amino acid sequence of SEQ ID NO: 113. In some embodiments, within any one of SEQ ID NO: 92-111, 114-116 and 124-126 the CH2 and CH3 can contain a mutation that decreases effector function.

It will be understood that though specific linkers are provided in the above-described molecules any linker can be used. In some embodiments, any flexible linker can be used. In some embodiments, the linker is a (GGGGS)6 linker. In some embodiments, the linker between two subunits is a (GGGGS)6 linker. In some embodiments, the polypeptide comprises or consists of the amino acid sequence of SEQ ID NO: 117. In some embodiments, the linker is a (GGGGS)3 linker. In some embodiments, the linker to the CH2 domain is a (GGGGS)3 linker. In some embodiments, the polypeptide comprises or consists of the amino acid sequence of SEQ ID NO: 118. In some embodiments, the polypeptide comprises or consists of the amino acid sequence of SEQ ID NO: 127. In some embodiments, the polypeptide comprises or consists of the amino acid sequence of SEQ ID NO: 128. In some embodiments, the polypeptide comprises or consists of the amino acid sequence of SEQ ID NO: 129. In some embodiments, the linker is a (GGGGS)6GS linker. In some embodiments, the linker between two subunits is a (GGGGS)6GS linker. In some embodiments, the polypeptide comprises or consists of the amino acid sequence of SEQ ID NO: 119. In some embodiments, any flexible linker can be used. In some embodiments, the linker is a (GGGGS)5 linker. In some embodiments, the linker between two subunits is a (GGGGS)5 linker. In some embodiments, the polypeptide comprises or consists of the amino acid sequence of SEQ ID NO: 120.

In some embodiments, the linker is a rigid linker. In some embodiments, the linker between the two subunits is a rigid linker. In some embodiments, the linker is a (EAAAK)2GGS linker. In some embodiments, the linker between two subunits is a (EAAAK)2GGS linker. In some embodiments, the polypeptide comprises or consists of the amino acid sequence of SEQ ID NO: 121. In some embodiments, the polypeptide comprises or consists of the amino acid sequence of SEQ ID NO: 122. In some embodiments, the polypeptide comprises or consists of the amino acid sequence of SEQ ID NO: 123.

In some embodiments, a polypeptide chain comprises a CH2 and CH3 domain is linked via a GS linker to a fragment of AChRg comprising a mutation to increase solubility and at least one mutation to decrease aggregation and linked via a GS linker to AChRa comprising a mutation to increase solubility and at least one mutation to decrease aggregation. In some embodiments, the polypeptide comprises or consists of the amino acid sequence of SEQ ID NO: 124. In some embodiments, a polypeptide chain comprises a CH2 and CH3 domain is linked via a GS linker to a fragment of AChRa comprising a mutation to increase solubility and at least one mutation to decrease aggregation and linked via a GS linker to AChRg comprising a mutation to increase solubility and at least one mutation to decrease aggregation. In some embodiments, the polypeptide comprises or consists of the amino acid sequence of SEQ ID NO: 125.

In some embodiments, the protein complex comprises a first polypeptide comprising or consisting of SEQ ID NO: 66 and a second polypeptide comprising or consisting of SEQ ID NO: 67. It will be understood by a skilled artisan that in this embodiment the polypeptide comprising AChRb contains the T366W mutation and the polypeptide comprising AChRg contains T366S/L368A/Y407V, but that the mutations could be switched to the opposite chains and the molecule would still be operable (see for example SEQ ID NO: 69). In some embodiments, the protein complex comprises a first polypeptide chain comprising or consisting of SEQ ID NO: 66 and a second polypeptide chain comprising or consisting of SEQ ID NO: 64. In some embodiments, the protein complex comprises a first polypeptide chain comprising or consisting of SEQ ID NO: 67 and a second polypeptide chain comprising or consisting of SEQ ID NO: 64. In some embodiments, the protein complex further comprises a first polypeptide comprising or consisting of SEQ ID NO: 66, a second polypeptide comprising or consisting of SEQ ID NO: 67 and a third polypeptide chain comprising or consisting of SEQ ID NO: 64. In some embodiments, the protein complex further comprises a fourth polypeptide chain comprising of consisting of SEQ ID NO: 64.

In some embodiments, a polypeptide chain comprises a fragment of AChRe comprising a mutation to increase solubility and linked via a linker to a heavy chain comprising CH1, hinge, CH2 and CH3 domains, wherein the CH3 domain comprises a mutation that inhibits homodimerization of the polypeptide chain. In some embodiments, the mutation that inhibits homodimerization is T366S, L368A and Y407V. In some embodiments, the polypeptide comprises or consists of the amino acid sequence

(SEQ ID NO: 68)
KNEELRLYHHLFNNYDPGSRPVREPEDTVTISLKVTLTNLISLNEKEET
LTTSVWIGIDWQDYRLNYSKDDFGGIETLRVPSELVWLPEIVLENNIDG
QFGVAYDANVLVYEGGSVTWLPPAIYRSVCDVSGVDTESGATNCSLIFR
SQTYNAEEVEFTFAVDNDGKTINKIDIDTEAYTENGEWAIDFCPGVIRR
HHGGATDGPGETDVIYSLIIRRKPGGGGSGGGGSGGGGSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLSCAVKGF
YPSDIAVEWESNGQPENNYKTTPPVLDSDGSFFLVSKLTVDKSRWQQGN
VFSCSVMHEALHNHYTQKSLSLSPGK.

In some embodiments, the protein complex comprises a first polypeptide comprising or consisting of SEQ ID NO: 66 and a second polypeptide comprising or consisting of SEQ ID NO: 68. It will be understood by a skilled artisan that in this embodiment the polypeptide comprising AChRb contains the T366W mutation and the polypeptide comprising AChRe contains T366S/L368A/Y407V, but that the mutations could be switched to the opposite chains and the molecule would still be operable. In some embodiments, the protein complex comprises a first polypeptide chain comprising or consisting of SEQ ID NO: 68 and a second polypeptide chain comprising or consisting of SEQ ID NO: 64. In some embodiments, the protein complex further comprises a first polypeptide comprising or consisting of SEQ ID NO: 66, a second polypeptide comprising or consisting of SEQ ID NO: 68 and a third polypeptide chain comprising or consisting of SEQ ID NO: 64. In some embodiments, the protein complex further comprises a fourth polypeptide chain comprising of consisting of SEQ ID NO: 64.

In some embodiments, a polypeptide chain comprises a fragment of AChRg comprising a mutation to increase solubility and linked via a linker to a heavy chain comprising CH1, hinge, CH2 and CH3 domains, wherein the CH3 domain comprises a mutation that inhibits homodimerization of the polypeptide chain. In some embodiments, the mutation that inhibits homodimerization is T366W. In some embodiments, the polypeptide comprises or consists of the amino acid sequence

(SEQ ID NO: 69)
RNQEERLLADLMQNYDPNLRPAERDSDVVNVSLKLTLTNLISLNEREEA
LTTNVWIEMQWCDYRLRWDPRDYEGLWVLRVPSTMVWRPDIVLENNVDG
VFEVALYCNVLVSPDGCIYWLPPAIFRSACDVSGVDTESGATNCSLIFQ
SQTYSTNEIDLQLSQEDGQTIEWIFIDPEAFTENGEWAIQHRPAKMLLD
PAAPAQEAGHQKVVFYLLIQRKPGGGGSGGGGSGGGGSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLWCLVKGFY
PSDIAVEWESNGQPENNYKTTPPVLDSDGSFFLYSKLTVDKSRWQQGNV
FSCSVMHEALHNHYTQKSLSLSPGK.

In some embodiments, the protein complex comprises a first polypeptide comprising or consisting of SEQ ID NO: 67 and a second polypeptide comprising or consisting of SEQ ID NO: 69. It will be understood by a skilled artisan that such a molecule could also be made using CH3 domains without the mutations that that inhibit homodimerization. In such a case only a single polypeptide chain would be needed as it would homodimerize. This polypeptide would be similar to SEQ ID NO: 65 but would include the AChRg fragment in place of the AChRb fragment.

In some embodiments, the protein complex comprises a first polypeptide comprising or consisting of SEQ ID NO: 69 and a second polypeptide comprising or consisting of SEQ ID NO: 68. It will be understood by a skilled artisan that in this embodiment the polypeptide comprising AChRg contains the T366W mutation and the polypeptide comprising AChRe contains T366S/L368A/Y407V, but that the mutations could be switched to the opposite chains and the molecule would still be operable. In some embodiments, the protein complex comprises a first polypeptide chain comprising or consisting of SEQ ID NO: 69 and a second polypeptide chain comprising or consisting of SEQ ID NO: 64. In some embodiments, the protein complex further comprises a first polypeptide comprising or consisting of SEQ ID NO: 69, a second polypeptide comprising or consisting of SEQ ID NO: 68 and a third polypeptide chain comprising or consisting of SEQ ID NO: 64. In some embodiments, the protein complex further comprises a fourth polypeptide chain comprising of consisting of SEQ ID NO: 64.

In some embodiments, a polypeptide chain comprises or consists of a sequence with at least 70% identity to a sequence provided herein. In some embodiments, a polypeptide chain comprises or consists of a sequence with at least 75% identity to a sequence provided herein. In some embodiments, a polypeptide chain comprises or consists of a sequence with at least 80% identity to a sequence provided herein. In some embodiments, a polypeptide chain comprises or consists of a sequence with at least 85% identity to a sequence provided herein. In some embodiments, a polypeptide chain comprises or consists of a sequence with at least 90% identity to a sequence provided herein. In some embodiments, a polypeptide chain comprises or consists of a sequence with at least 95% identity to a sequence provided herein. In some embodiments, a polypeptide chain comprises or consists of a sequence with at least 97% identity to a sequence provided herein. In some embodiments, a polypeptide chain comprises or consists of a sequence with at least 99% identity to a sequence provided herein.

In some embodiments, the protein is selected from any one of SEQ ID NO: 72-91. In some embodiments, the polypeptide is selected from any one of SEQ ID NO: 72-91. In some embodiments, the polypeptide is selected from any one of SEQ ID NO: 92-100 and 102-130. In some embodiments, the polypeptide is selected from any one of SEQ ID NO: 72-100 and 102-130. In some embodiments, the polypeptide is selected from any one of SEQ ID NO: 94, 104, and 108-129.

### Pharmaceutical Compositions

By another aspect, there is provided a pharmaceutical composition comprising a protein of the invention.

By another aspect, there is provided a pharmaceutical composition comprising a polypeptide chain of the invention.

By another aspect, there is provided a pharmaceutical composition comprising a protein complex of the invention.

By another aspect, there is provided a pharmaceutical composition comprises a composition of the invention.

In some embodiments, the pharmaceutical composition comprises a pharmaceutically acceptable carrier, excipient or adjuvant. As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is for use in treating myasthenia gravis. In some embodiments, the pharmaceutical composition comprises a therapeutically effective amount of the protein complex of the invention. In some embodiments, the pharmaceutical composition comprises a therapeutically effective amount of the conjugate of the invention. The term “therapeutically effective amount” refers to an amount of a drug effective to treat a disease or disorder in a mammal. In some embodiments, a therapeutically effective amount is an amount effective, at dosages and for periods of time necessary, to achieve the desired therapeutic or prophylactic result. The exact dosage form and regimen would be determined by the physician according to the patient's condition. In some embodiments, an effective amount is an amount sufficient to treat at least one symptom of a disease. In some embodiments, the disease is myasthenia gravis. In some embodiments, myasthenia gravis is characterized by autoantibodies against the protein.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life. Treatment of myasthenia gravis is well known in the art and may include any acceptable measure for assessing improvement of a myasthenia gravis symptom. This may include, improved muscle control, reduced muscle drooping, lapping or heaviness, improved breathing, reduced autoantibody titer, improved synapsis function or any other measure of improvement.

In some embodiments, the pharmaceutical composition is formulated for systemic administration. In some embodiments, the pharmaceutical composition is formulated for administration to a subject. In some embodiments, the pharmaceutical composition is formulated for administration to a human. In some embodiments, the pharmaceutical composition is formulated for intravenous administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal. In some embodiments, the administering is intravenous administering. In some embodiments, the administering is selected from oral, intravenous, intramuscular, intraperitoneal, intertumoral, topical, or subdermal administration. In some embodiments, administering is administering to a site of disease.

The dosage administered will be dependent upon the age, health, and weight of the recipient, kind of concurrent treatment, if any, frequency of treatment, and the nature of the effect desired.

### Methods of Treatment

By another aspect, there is provided a method of treating myasthenia gravis in a subject in need thereof, the method comprising administering to the subject a protein of the invention, thereby treating myasthenia gravis in a subject.

By another aspect, there is provided a method of treating myasthenia gravis in a subject in need thereof, the method comprising administering to the subject a polypeptide chain of the invention, thereby treating myasthenia gravis in a subject.

By another aspect, there is provided a method of treating myasthenia gravis in a subject in need thereof, the method comprising administering to the subject a protein complex of the invention, thereby treating myasthenia gravis in a subject.

By another aspect, there is provided a method of treating myasthenia gravis in a subject in need thereof, the method comprising administering to the subject a composition of the invention, thereby treating myasthenia gravis in a subject.

In some embodiments, the administering is administering a pharmaceutical composition of the invention. In some embodiments, myasthenia gravis is characterized by antibodies against the protein. In some embodiments, the protein is a target of myasthenia gravis antibodies. It will be understood by the skilled artisan that a protein complex will be designed with fragments of proteins which are targeted by myasthenia gravis antibodies in the subject. In some embodiments, antibodies are autoantibodies.

In some embodiments, treating comprises lowering antibody concentration. In some embodiments, treating comprises lower antibody number. In some embodiments, antibody concentration is circulating antibody concentration. In some embodiments, treating comprises depleting antibodies. In some embodiments, treating comprises sequestering antibodies. In some embodiments, binding of the antibodies to the molecules of the invention result in sequestering of the antibodies. In some embodiments, treating comprises killing B cells. In some embodiments, the B cell are autoreactive B cells. In some embodiments, killing B cells is specific B cell killing. In some embodiments, treating comprises killing B cells that produce the antibodies. In some embodiments, treating comprises killing B cells that produce the antibodies and the not substantially killing other B cells. In some embodiments, treating comprises killing B cell that produce antibodies against the protein complex. In some embodiments, treating comprises killing B cell that produce antibodies against the fragment. In some embodiments, treating comprises killing B cell that produce antibodies against a fragment of the protein complex.

In some embodiments, lowering antibodies comprises binding antibodies. In some embodiments, lowering is removing at least 10, 20, 25, 30, 40, 50, 60, 70, 75, 80, 90, 95, 97, 99 or 100% of the antibodies. Each possibility represents a separate embodiment of the invention. In some embodiments, antibodies are autoantibodies. In some embodiments, antibodies in antibodies in the subject. In some embodiments, antibodies are circulating antibodies. In some embodiments, autoantibodies are autoantibodies against the protein or fragment. In some embodiments, autoantibodies are cytotoxic autoantibodies. In some embodiments, autoantibodies comprise IgG1 autoantibodies. In some embodiments, autoantibodies comprise IgG3. In some embodiments, autoantibodies comprise IgG1 and IgG3 autoantibodies. In some embodiments, autoantibodies comprise IgG1, IgG2 and IgG3 autoantibodies. In some embodiments, autoantibodies comprise IgG1, IgG3 and IgG4 autoantibodies. In some embodiments, autoantibodies comprise IgG1, IgG2, IgG3 and IgG4 autoantibodies. In some embodiments, lowering is removing at least 25% of the antibodies. In some embodiments, lowering is removing at least 50% of the antibodies. In some embodiments, lowering is removing at least 70% of the antibodies. In some embodiments, lowering is removing at least 75% of the antibodies. In some embodiments, percent of the antibodies is percent of the autoantibodies. In some embodiments, percent of the antibodies is percent of the antibodies against the protein or fragment. In some embodiments, percent of the antibodies is percent of the antibodies associated with the disease.

In some embodiments, the method further comprises reducing antibodies in the subject. In some embodiments, the reducing is before the administering. In some embodiments, the reducing antibodies is reducing circulating antibodies. In some embodiments, the antibodies are autoantibodies. In some embodiments, the antibodies are against a protein. In some embodiments, the antibodies are against the protein that the fragment is from. In some embodiments, the antibodies are against the protein that at least one of the fragments is from. In some embodiments, the reducing is reducing antibodies against all proteins that at least one of the fragments are from. In some embodiments, the antibodies are against the protein complex. Methods of reducing antibodies are well known in the art and include, for example, plasmapheresis, intravenous Ig (IVIg), antibody filtering, and B cell targeting therapies, any of which may be employed. In some embodiments, the method comprises plasmapheresis of the antibodies before administering. In some embodiments, the method comprises administering a B cell targeting therapy before administering the therapeutic of the invention. In some embodiments, a B cell targeting therapy is an anti-B cell therapy. In some embodiments, the B cell targeting therapy is B cell lethal therapy. In some embodiments, the B cell targeting therapy is a pan B cell therapy. In some embodiments, the B cell targeting therapy is not a targeted therapy. As used herein, a “targeted B cell therapy” is a therapy that targets only specific B cell clones that produce specific antibodies. In some embodiments, an anti-B cell therapy is an anti-B cell antibody. B cell targeting antibodies are known in the art and include for non-limiting example, anti-CD20 antibodies. Anti-CD20 therapeutic antibodies are well known in the art and include, but are not limited to rituximab, ocrelizumab, obinutuzumab, ofatumumab, ibritumomab, tiuxetan, tositumomab, and ublituximab. In some embodiments, the B cell targeting therapy is rituximab.

### Nucleic Acids

By another aspect, there is provided a nucleic acid molecule encoding a protein of the invention.

By another aspect, there is provided a nucleic acid system comprising at least two nucleic acid molecules, wherein a first nucleic acid molecule encodes the first polypeptide chain of a protein complex of the invention and a second nucleic acid molecules encodes the second polypeptide chain of the protein complex of the invention.

By another aspect, there is provided a nucleic acid system comprising at least two nucleic acid molecules, wherein a first nucleic acid molecule encodes a first polypeptide chain comprising a fragment of a first human protein target of myasthenia gravis autoantibodies or an analog or derivative thereof and a first dimerization domain and a second nucleic acid molecule encodes a second polypeptide chain comprising a fragment of a second human protein target of myasthenia gravis autoantibodies or an analog or derivative thereof and second dimerization domain.

By another aspect, there is provided a nucleic acid molecule encoding a polypeptide chain of a composition of the invention.

By another aspect, there is provided a nucleic acid molecule encoding a composition of the invention.

By another aspect, there is provided a nucleic acid molecule encoding a fragment of a first protein target of myasthenia gravis autoantibodies or an analog or derivative thereof and fragment of a second human protein target of myasthenia gravis autoantibodies or an analog or derivative thereof.

In some embodiments, the nucleic acid molecule is for use in treating myasthenia gravis. In some embodiments, the nucleic acid system is for use in treating myasthenia gravis.

In some embodiments, the nucleic acid system further comprises a third nucleic acid molecule that encodes a third polypeptide of the protein complex of the invention. In some embodiments, the nucleic acid system further comprises a fourth nucleic acid molecule that encodes a fourth polypeptide of the protein complex of the invention. In some embodiments, a first nucleic acid molecule encodes the first polypeptide of the invention. In some embodiments, a second nucleic acid molecule encodes the second polypeptide of the invention. In some embodiments, a third nucleic acid molecule encodes the third polypeptide of the invention. In some embodiments, a fourth nucleic acid molecule encodes the fourth polypeptide.

In some embodiments, the nucleic acid molecule is a vector. In some embodiments, the vector is an expression vector. In some embodiments, nucleic acid molecule comprises an open reading frame encoding the polypeptide chain. Expressing of an open reading frame within a cell is well known to one skilled in the art. It can be carried out by, among many methods, transfection, viral infection, or direct alteration of the cell's genome. Expression vectors are well known in the art and any vector compatible with a target cell in which the protein complex of the invention is being expressed may be used.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence. In some embodiments, the vector comprises a promoter. In some embodiments, the promoter is configured for expression in a target cell in which the protein complex of the invention is being expressed.

The vector may be a DNA plasmid delivered via non-viral methods or via viral methods. The viral vector may be a retroviral vector, a herpesviral vector, an adenoviral vector, an adeno-associated viral vector or a poxviral vector. The promoter may be active in mammalian cells. The promoters may be a viral promoter. The promoter may be active in bacterial cells. The promoter may be active in human cells. The promoter may be active in fibroblasts. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, the open reading frame is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the nucleic acid molecule is a single nucleic acid molecule. In some embodiments, the first and second nucleic acid molecules are different molecules. In some embodiments, the first and second nucleic acid molecule are the same molecule. In some embodiments, any two of the first, second, third and fourth nucleic acid molecules are different molecules. In some embodiments, any two of the first, second, third and fourth nucleic acid molecules are the same molecule. In some embodiments, any three of the first, second, third and fourth nucleic acid molecules are different molecules. In some embodiments, the first, second, and third nucleic acid molecules are different molecules. In some embodiments, any three of the first, second, third and fourth nucleic acid molecules are the same molecule. In some embodiments, all of the first, second, third and fourth nucleic acid molecules are different molecules. In some embodiments, all of the first, second, third and fourth nucleic acid molecules are the same molecule.

### Methods of Production

By another aspect, there is provided a method for producing a protein, the method comprising:
obtaining a first fragment of an extracellular domain of a first human receptor or an analog or derivative thereof, and a second fragment of an extracellular domain of a second human receptor or analog or derivative thereof, wherein the first and second human receptors are targets of myasthenia gravis autoantibodies and different proteins and linking the first fragment to the second fragment to produce a single polypeptide chain;
thereby producing a protein.

By another aspect, there is provided a method for producing a protein, the method comprising:
obtaining a first fragment of a human receptor target of myasthenia gravis autoantibodies and generating in the first fragment at least one mutation that decreases aggregation of the first fragment;
thereby producing a protein.

By another aspect, there is provide a method for producing a protein complex, the method comprising:
obtaining a first fragment of a first protein target of myasthenia gravis autoantibodies or an analog or derivative thereof and a second fragment of a second protein target of myasthenia gravis autoantibodies or an analog or derivative thereof, linking the first fragment to a first dimerization domain to produce a first polypeptide and linking the second fragment to a second dimerization domain to produce a second polypeptide chain;
thereby producing a protein complex.

By another aspect, there is provided a method for producing a protein, the method comprising:
culturing a host cell comprising one or more vectors comprising a nucleic acid sequence encoding a single polypeptide chain, wherein the single polypeptide chain is produced by:
   i. obtaining a first fragment of a human receptor target of myasthenia gravis autoantibodies; and
   ii. generating in the first fragment at least one mutation that decreases aggregation of the first fragment;
thereby producing a protein.

By another aspect, there is provided a method for producing a protein, the method comprising:
culturing a host cell comprising one or more vectors comprising a nucleic acid sequence encoding a single polypeptide chain, wherein the single polypeptide chain is produced by:
   i. obtaining a first fragment of an extracellular domain of a first human receptor or an analog or derivative thereof and a second fragment of an extracellular domain of a second human receptor or analog or derivative thereof, wherein the first and second human receptors are targets of myasthenia gravis autoantibodies and are different proteins; and
   ii. linking the first fragment to the second fragment to produce a single polypeptide chain;
thereby producing a protein.

By another aspect, there is provide a method for producing a protein complex, the method comprising:
culturing a host cell comprising one or more vectors comprising a nucleic acid sequence encoding at least two polypeptide chains, wherein the two polypeptide chains are produced by:
   i. obtaining a first fragment of a first protein target of myasthenia gravis autoantibodies or an analog or derivative thereof and a second fragment of second protein target of myasthenia gravis autoantibodies or analog or derivative thereof; and
   ii. linking the first fragment to a first dimerization domain to produce a first polypeptide chain and linking the second fragment to a second dimerization domain to produce a second polypeptide chain;
thereby producing a protein complex.

In some embodiments, the protein is a polypeptide. In some embodiments, the protein complex is a protein complex of the invention. In some embodiments, the protein composition is a composition of the invention. In some embodiments, the protein is a protein of the invention. In some embodiments, the protein is a polypeptide chain of the invention. In some embodiments, the fragment is a fragment of the invention. In some embodiments, the derivative is a derivative of the invention. In some embodiments, the analog is an analog of the invention. In some embodiments, the dimerization domain is a dimerization domain of the invention. In some embodiments, the composition, protein complex, protein, fragment, analog, derivative or dimerization domain is such as is described hereinabove.

In some embodiments, the protein is a human protein. In some embodiments, the protein is a cell surface protein. In some embodiments, the first and second protein are the same protein. In some embodiments, the first and second protein are different proteins. In some embodiments, the first and second proteins are targets of myasthenia gravis autoantibodies. In some embodiments, the first and second proteins are targets of autoantibodies associated with myasthenia gravis. In some embodiments, myasthenia gravis is characterized by autoantibodies against the first and second proteins. In some embodiments, the protein is a receptor, and the fragment is a fragment of the extracellular domain. In some embodiments, the fragment comprises a fragment of the extracellular domain. In some embodiments, the fragment consists of the extracellular domain.

In some embodiments, the first and second dimerization domains are capable of dimerizing to each other. In some embodiments, the first and second dimerization domains are configured to dimerize with each other. In some embodiments, the method further comprises contacting the first and second polypeptides. In some embodiments, the contacting comprises incubating the polypeptides together. In some embodiments, the contacting is in a cell. In some embodiments, the contacting is in vitro. In some embodiments, the contacting is under conditions sufficient to allow dimerization. In some embodiments, allowing is inducing. In some embodiments, the conditions are sufficient to allow dimerization of the polypeptides. In some embodiments, the conditions are physiological conditions.

In some embodiments, the method further comprises inserting a third dimerization domain into the first polypeptide. In some embodiments, inserting is linking. In some embodiments, inserting is inserting a nucleic acid sequence encoding the third dimerization domain into a nucleic acid molecule or vector encoding the first polypeptide. In some embodiments, the linking is linking the third dimerization domain to the first dimerization domain. In some embodiments, the linking is linking the third dimerization domain to the first fragment.

In some embodiments, the method further comprises obtaining a third fragment of a third protein target of myasthenia gravis autoantibodies or an analog or derivative thereof and linking it to a fourth dimerization domain to produce a third polypeptide chain. In some embodiments, the third and fourth dimerization domains are capable of dimerization to each other. In some embodiments, the third and fourth dimerization domains are configured to dimerize to each other. In some embodiments, the method further comprises contacting the first, second and third polypeptide chains. In some embodiments, the method further comprises expressing in the host cell a nucleic acid sequence encoding a third polypeptide chain. In some embodiments, the third polypeptide chain is produced by obtaining a third fragment of a third protein and linking it to a fourth dimerization domain to produce a third polypeptide chain. In some embodiments, the method comprises expression the first, second and third polypeptide chains in a cell.

In some embodiments, the method further comprises inserting a fifth dimerization domain into the second polypeptide. In some embodiments, inserting is linking. In some embodiments, inserting is inserting a nucleic acid sequence encoding the fifth dimerization domain into a nucleic acid molecule or vector encoding the second polypeptide. In some embodiments, the linking is linking the fifth dimerization domain to the second dimerization domain. In some embodiments, the linking is linking the fifth dimerization domain to the second fragment.

In some embodiments, the method further comprises obtaining a fourth fragment of a fourth protein target of myasthenia gravis autoantibodies or an analog or derivative thereof and linking it to a sixth dimerization domain to produce a fourth polypeptide chain. In some embodiments, the fifth and sixth dimerization domains are capable of dimerization to each other. In some embodiments, the fifth and sixth dimerization domains are configured to dimerize to each other. In some embodiments, the method further comprises contacting the first, second, third and fourth polypeptide chains. In some embodiments, the method further comprises expressing in the host cell a nucleic acid sequence encoding a fourth polypeptide chain. In some embodiments, the fourth polypeptide chain is produced by obtaining a fourth fragment of a fourth protein and linking it to a sixth dimerization domain to produce a fourth polypeptide chain. In some embodiments, the method comprises expression the first, second, third and fourth polypeptide chains in a cell.

In some embodiments, the method further comprises inserting an Fc region into the first chain. In some embodiments, the method further comprises inserting an Fc region into the second chain. In some embodiments, the method further comprises inserting an Fc region into the third chain. In some embodiments, the method further comprises inserting an Fc region into the fourth chain. In some embodiments, the method further comprises inserting a portion of an Fc region into the first chain and a portion of the Fc region into the second chain wherein and interface of the two portions produces a complete Fc region.

In some embodiments, an Fc region is inserted C-terminally to a dimerization domain. In some embodiments, an Fc region is inserted C-terminally to a fragment. In some embodiments, an Fc region is inserted N-terminally to a dimerization domain. In some embodiments, an Fc region is inserted N-terminally to a fragment. In some embodiments, a fragment is inserted or linked C-terminally to a dimerization domain. In some embodiments, a fragment is inserted or linked N-terminally to a dimerization domain.

In some embodiments, the method further comprises inserting a linker between at least two sections of a polypeptide chain. In some embodiments, the linker is inserted between a fragment and a dimerization domain. In some embodiments, the linker is inserted between a fragment and an Fc region. In some embodiments, the linker is inserted between an Fc region and a dimerization domain. In some embodiments, the linker is inserted between a dimerization domain and another dimerization domain. In some embodiments, the linker is inserted between a fragment and another fragment. In some embodiments, the linker is inserted between a fragment of a first protein and a fragment of a second protein.

In some embodiments, the method further comprises producing at least one mutation in the fragment that increases solubility of the fragment. In some embodiments, the method further comprises measuring solubility of the mutated fragment. In some embodiments, the method further comprises selecting a mutated fragment with increased solubility. In some embodiments, increasing is increasing by at least a predetermined threshold. In some embodiments, increasing is significantly increasing. In some embodiments, increasing is by at least 10, 15, 20, 25, 30, 35, 40, 45, 50, 55, 60, 65, 70, 75 80, 85, 90, 95, 100, 150, 200, 250, 300, 350, 400, 450 or 500%. Each possibility represents a separate embodiment of the invention. In some embodiments, increasing is by at least 25%. In some embodiments, increasing is by at least 50%.

In some embodiments, the method further comprises producing at least one mutation in the fragment that decreases aggregation of the fragment. In some embodiments, the method further comprises measuring aggregation of the mutated fragment. In some embodiments, the method further comprises selecting a mutated fragment with decreased solubility. In some embodiments, decreasing is decreasing by at least a predetermined threshold. In some embodiments, decreasing is significantly decreasing. In some embodiments, decreasing is by at least 10, 15, 20, 25, 30, 35, 40, 45, 50, 55, 60, 65, 70, 75 80, 85, 90, 95, 100, 150, 200, 250, 300, 350, 400, 450 or 500%. Each possibility represents a separate embodiment of the invention. In some embodiments, decreasing is by at least 25%. In some embodiments, decreasing is by at least 50%.

In some embodiments, the method further comprises confirming the at least one mutation does not substantially decrease binding of the first fragment to autoantibodies against the first fragment. In some embodiments, the method further measuring binding of autoantibodies to the mutated first fragment. In some embodiments, the method comprises selecting a mutated fragment comprising substantially the same or more autoantibody binding. In some embodiments, autoantibodies and myasthenia gravis autoantibodies. In some embodiments, the autoantibodies are autoantibodies found in myasthenia gravis subjects. In some embodiments, the confirming or measuring comprises contacting the mutated fragment with sample from a subject suffering from myasthenia gravis and measuring binding of autoantibodies in the sample to the mutated fragment. In some embodiments, the sample is blood. In some embodiments, the sample is sera. In some embodiments, the sample comprises isolated autoantibodies. In some embodiments, the confirming or measuring comprises contacting the unmutated fragment with sample from a subject suffering from myasthenia gravis and measuring binding of autoantibodies in the sample to the mutated fragment. In some embodiments, the confirming or measuring comprises comparing the binding of the unmutated fragment to the mutated fragment and selecting a mutated fragment that does not have substantially less autoantibody binding. In some embodiments, substantially less is significantly less. In some embodiments, substantially less is less. In some embodiments, substantially less is more than 5, 10, 15, 20, 25, 30, 35, 40, 45 or 50% less binding. Each possibility represents a separate embodiment of the invention. In some embodiments, substantially less is more 10% less binding.

By another aspect, there is provided a protein complex produced by a method of the invention.

By another aspect, there is provided a protein produced by a method of the invention.

By another aspect, there is provided a composition produced by a method of the invention.

### Patient Selection

By another aspect, there is provided a method of determining suitability of a subject to be treated by a method of the invention, the method comprising receiving a sample from the subject, contacting the sample with a composition of the invention and determining binding of antibodies within the sample to the composition, wherein binding of the antibodies to the composition indicates the subject is suitable to be treated by a method of the invention, thereby determining suitability of the subject to be treated.

By another aspect, there is provided a method of determining suitability of a subject to be treated by a method of the invention, the method comprising receiving a sample from the subject, contacting the sample with a protein complex of the invention and determining binding of antibodies within the sample to the protein complex, wherein binding of the antibodies to the protein complex indicates the subject is suitable to be treated by a method of the invention, thereby determining suitability of the subject to be treated.

By another aspect, there is provided a method of determining suitability of a subject to be treated by a method of the invention, the method comprising receiving a sample from the subject, contacting the sample with a protein of the invention and determining binding of antibodies within the sample to the protein, wherein binding of the antibodies to the protein indicates the subject is suitable to be treated by a method of the invention, thereby determining suitability of the subject to be treated.

In some embodiments, the subject is a subject in need thereof. In some embodiments, the subject is a subject such as described hereinabove. In some embodiments, the subject suffers from myasthenia gravis. In some embodiments, the subject is known to be positive for autoantibodies associated with myasthenia gravis. In some embodiments, the subject is seropositive. In some embodiments, the subject is seronegative. In some embodiments, the subject is naïve to treatment. In some embodiments, the treatment is treatment for myasthenia gravis. In some embodiments, the subject has received treatment and has relapsed.

In some embodiments, the method comprises obtaining the sample from the subject. In some embodiments, the sample comprises tissue. In some embodiments, the sample is a biopsy. In some embodiments, the sample is a bodily fluid. In some embodiments, the bodily fluid is blood. In some embodiments, the bodily fluid is serum. In some embodiments, the bodily fluid is plasma. In some embodiments, the bodily fluid is a fluid that comprises antibodies. In some embodiments, the bodily fluid is selected from at least one of: blood, serum, plasma, intestinal fluid, saliva, tumor fluid, urine, interstitial fluid, cerebral spinal fluid and stool.

In some embodiments, the autoantibodies are myasthenia gravis autoantibodies. In some embodiments, the autoantibodies are against AChR. In some embodiments, the autoantibodies are antibodies against AChR. In some embodiments, the autoantibodies are antibodies against an AChR subunit. In some embodiments, the autoantibodies are against an AChR subunit. In some embodiments, autoantibodies are pathologic autoantibodies. In some embodiments, the autoantibodies are disease causing autoantibodies

In some embodiments, contacting is incubating. In some embodiments, contacting is under conditions sufficient for binding of antibodies to the protein complex. In some embodiments, conditions comprise a time sufficient for binding of antibodies to the protein complex. In some embodiments, conditions comprise physiological conditions. In some embodiments, the protein complex is added to the sample. In some embodiments, the protein complex is dissolved in the bodily fluid. In some embodiments, the antibodies are autoantibodies. In some embodiments, the antibodies are antibodies against a protein.

In some embodiments, binding of at least a threshold amount of antibodies to the protein or protein complex indicates the subject is suitable for treatment. In some embodiments, binding of more than a threshold amount of antibodies to the protein or protein complex indicates the subject is suitable for treatment. In some embodiments, the amount of antibodies is the number of antibodies. In some embodiments, the amount of antibodies is the percentage of antibodies. In some embodiments, the percentage is the percentage of antibodies in the sample. In some embodiments, the threshold is 10, 15, 20, 25, 30, 35, 40, 45, 50, 55, 60, 65, 70, or 75% of antibodies in the sample. Each possibility represents a separate embodiment of the invention. In some embodiments, the threshold is 20%. In some embodiments, the threshold is 25%. In some embodiments, the threshold is 50%. In some embodiments, the threshold is 70%. In some embodiments, the threshold is 75%.

In some embodiments, the composition further comprises a detectable moiety. In some embodiments, the protein complex further comprises a detectable moiety. In some embodiments, the protein further comprises a detectable moiety. In some embodiments, the method further comprises contacting the composition, complex and/or protein with a peptide comprising a detectable moiety. In some embodiments, the peptide is configured to bind the composition, protein and/or complex. In some embodiments, the peptide is specific to the composition, protein and/or complex. As used herein, the term “specific binding” refers to binding to a specific molecule to the exclusion of other molecules. In some embodiments, the peptide is specific to the composition, protein and/or complex to the exclusion of other proteins in the sample. In some embodiments, the peptide is specific to the composition, protein and/or complex to the exclusion of naturally occurring antibodies in the sample. In some embodiments, the peptide is specific to the composition, protein and/or complex to the exclusion of the antibodies in the sample. In some embodiments, the determining binding comprises detecting the moiety. In some embodiments, the determining comprises isolating the protein complex. In some embodiments, the determining comprises eluting antibodies from the complex. Methods of protein identification are well known in the art and any such method may be used. Examples of such method include western blotting, ELISA, FACS analysis and protein sequencing, such as by mass spectrometry. In some embodiments, the determining comprises ELISA. In some embodiments, the ELISA is a competitive ELISA. In some embodiments, the competitive ELISA comprises competition with antibodies. In some embodiments, the antibodies are antibodies associated with the disease.

In some embodiments, binding is positive binding. In some embodiments, binding is binding above a predetermined threshold. In some embodiments, binding is specific binding. In some embodiments, binding is binding to at least one of the fragments of the protein complex. In some embodiments, binding is binding to at least two of the fragments of the protein complex. In some embodiments, binding is binding to at least three of the fragments of the protein complex. In some embodiments, binding is binding to at least four of the fragments of the protein complex. In some embodiments, binding of at least 10, 20, 25, 30, 40, 50, 60, 70, 75, 80, 90, 95, 97, 99 or 100% of the antibodies in the sample. Each possibility represents a separate embodiment of the invention. In some embodiments, binding of at least 50% of the antibodies in the sample. In some embodiments, binding of at least 70% of the antibodies in the sample. In some embodiments, binding of at least 75% of the antibodies in the sample. In some embodiments, percent of the antibodies is percent of the autoantibodies. In some embodiments, percent of the antibodies is percent of the antibodies against the protein. In some embodiments, percent of the antibodies is percent of the antibodies associated with the disease.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, immunological, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

80 to 90% of all Myasthenia Gravis (MG) patients are found to have autoantibodies against acetylcholine receptor (AChR). However, the acetylcholine receptor complex is made up of five subunits (alpha1, beta1, gamma, delta and epsilon). Diagnostic assessment of MG patients does not generally distinguish between antibodies against one subunit or the other. Nevertheless, most antigen specific attempts at generating MG therapeutics have focused on the alpha subunit of AChR (AChRa) and therapeutics that target autoantibodies against this molecule.

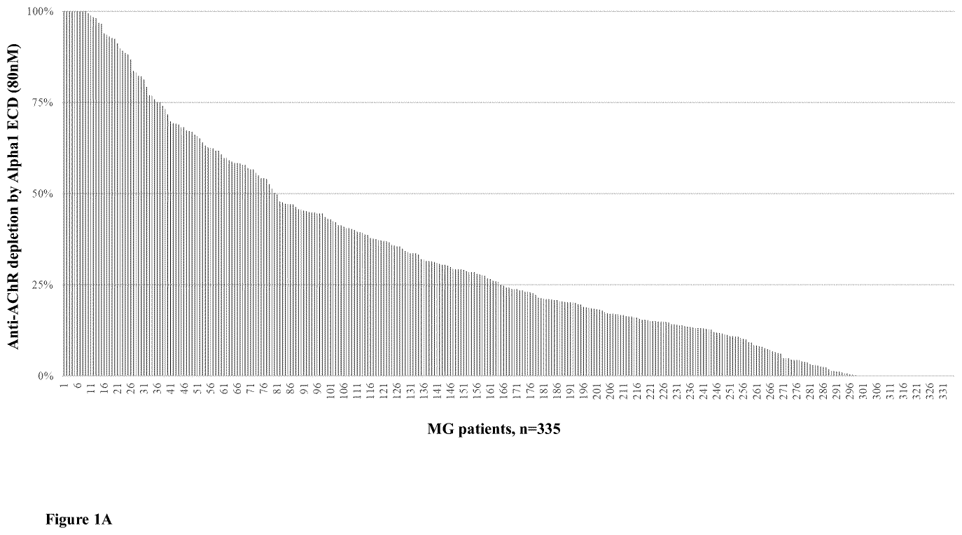

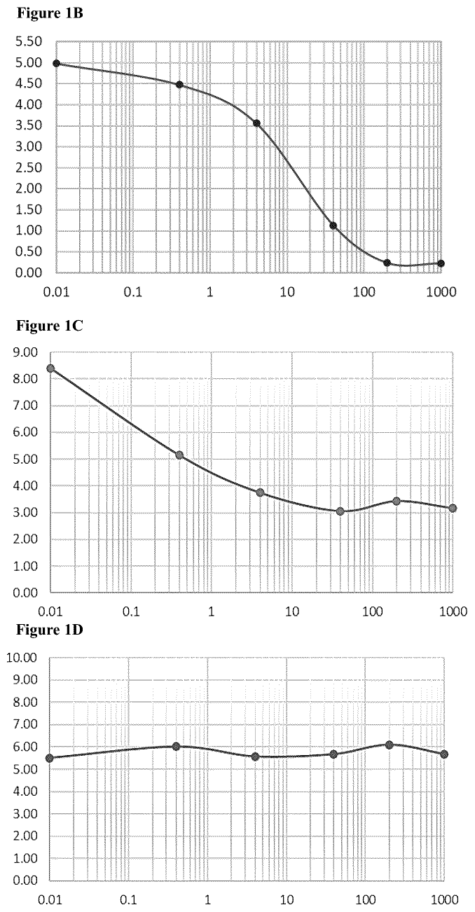

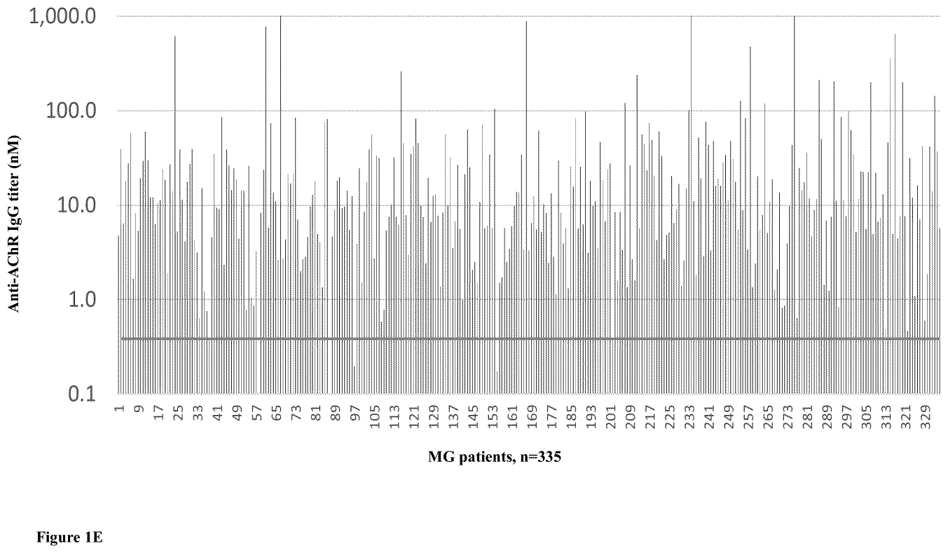

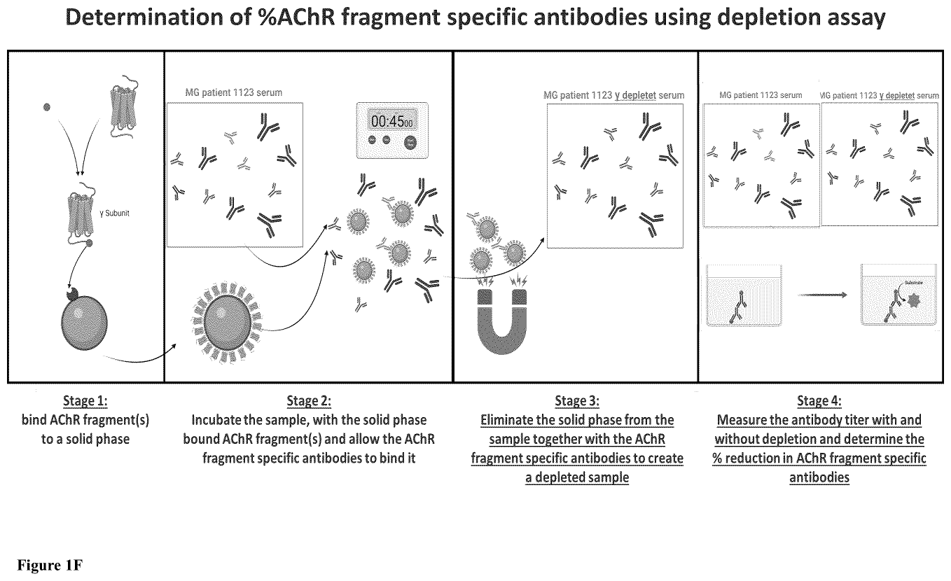

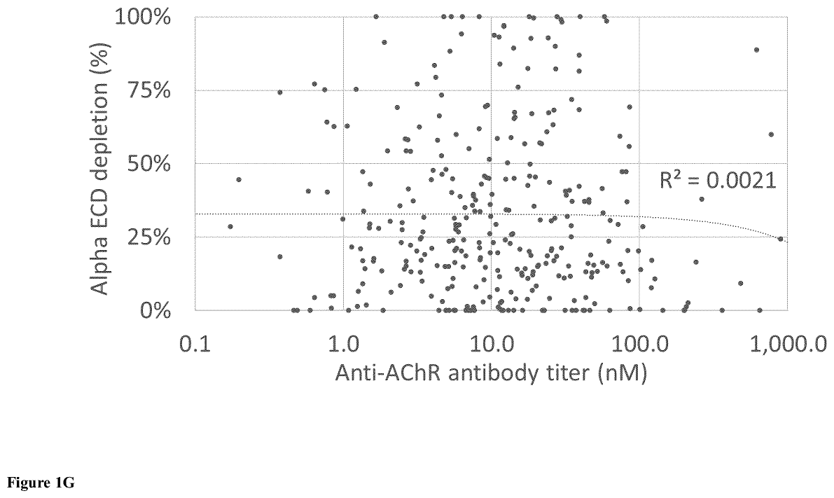

In order to determine the percentage of the Myasthenia Gravis population that actually has anti-AChRα1, serum samples were collected from 335 AChR-seropositive MG patients. The samples were tested in a direct ELISA assay, using a cis loop modified AChRα1 extracellular domain (ECD, SEQ ID NO: 131) as a decoy depleting molecule (Example for the process is described in FIG. 1F). For this assay, the full AChR complex, with all of its subunits, was used for bait to bind autoantibodies in the serum. This binding assay was performed with or without the presence of increasing concentration of solid phase bound AChRα1 and the percent reduction in binding was measured for each sample (FIG. 1A). The reduction in the AChR receptor binding is proportional to the concentration of autoantibodies present against AChRαl. Surprisingly, though some subjects had very high levels of inhibition, indicating the presence of predominantly autoantibodies against AChRα1 (FIG. 1A, left-most samples; and FIG. 1B), others showed only moderate levels of inhibition indicating that the majority of autoantibodies were not against the alpha subunit (FIG. 1C) and still others had no substantial inhibition indicating that though they were positive for autoantibodies against AChR, no more than 10% of their autoantibodies were against the AChRα1 (FIG. 1D). Importantly, when total AChR binding was measured (FIG. 1E) there was no correlation between the total antibody concentration and the percent of the antibodies that are anti-AChRα1 (FIG. 1G), several of the samples with the highest total antibody titer had low or absent anti-AChRα1 autoantibodies.

### Example 2

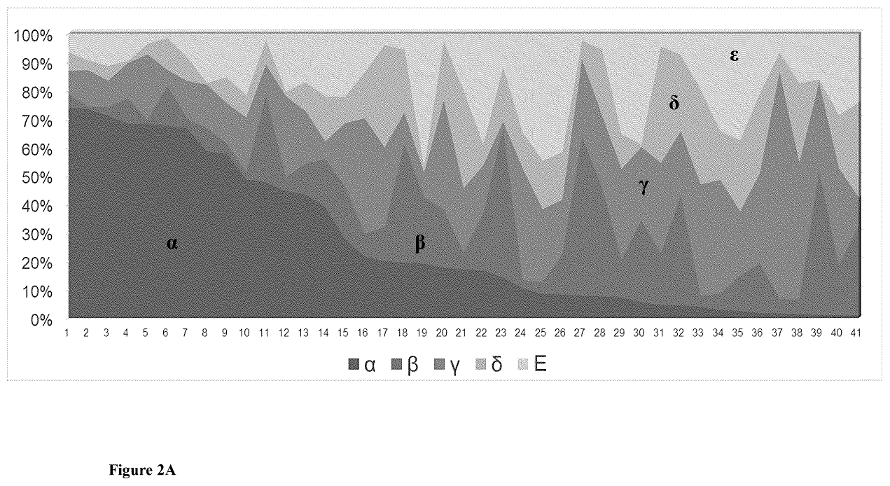

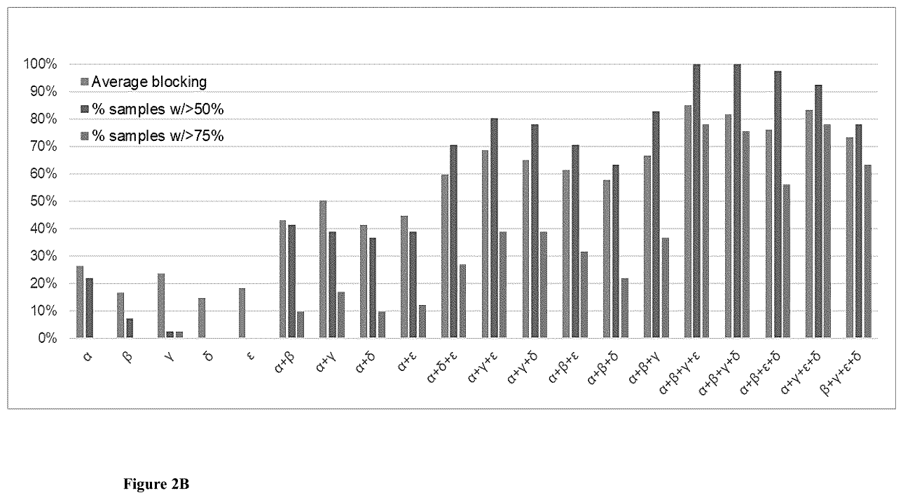

To better understand the autoantibody repertoire of most MG patients, an analysis was run on data provided in Zisimopoulou et al., 2008, “Antigen-specific apheresis of human anti-acetylcholine receptor autoantibodies from myasthenia gravis patients' sera using Escherichia coli-expressed receptor domains”. The 41 patient samples that were tested were mapped based on the contribution of autoantibodies against each AChR subunit to the total anti-AChR autoantibody pool (FIG. 2A). As can be seen, though autoantibodies against the alpha subunit contributes to many subjects, many others have autoantibodies predominantly against other subunits, and indeed the vast majority have a combination of autoantibodies targeting different subunits. Therefore, a plot was created showing the percent of subjects that would have at least a 50% or 75% inhibition by contacting with either a single AChR subunit or a combination of subunits (FIG. 2B). Surprisingly, the alpha subunit alone, and indeed any of the subunits alone, would rarely produce 75% blocking in any of the tested patients. Indeed, the alpha subunit alone would only produce greater than 50% inhibition in about 20% of patients.

In order for an MG therapeutic to be able to treat at least 50% of the target population and neutralize over 50% and ideally over 75% of the autoantibodies a combination of alpha/beta/gamma/delta/epsilon would be needed (FIG. 2B). While potentially any reduction in the levels of autoantibodies would be beneficial, to produce a treatment that could make a substantial reduction and be effective for a large percentage of the MG population, multiple AChR subunits need to be targeted. This can also be accomplished with double and triple combinations of these subunits.

### Example 3

Long term remission for MG patients would need to remove the majority of autoreactive B cells which produce the autoantibody pool. Simply removing the autoantibodies from circulation, while potentially effective in treating the symptoms of MG, would require repeated treatments for the rest of the subject's life as the long-lived B cells would perpetually continue to make new autoantibodies. Importantly, the B cells that produced the autoantibodies express B-cell receptor (BCR) on their surfaces which is identical to the autoantibodies. This allows the B cells themselves to be targeted by a therapeutic that contains the BCR- (and autoantibody) specific epitope. By coupling the target epitope to the Fc region of the antibody heavy chain, a therapeutic can direct specific killing of autoantibody producing B cells. This approach is also robust to potential evasion of specific subpopulations, which occurs when using agents that are targeting specific differentiation markers on the cell surface (e.g., CD19, CD38, BCMA), as every cell carrying the autoreactive BCR will be targeted regardless of its differentiations state. This approach is also beneficial in protecting and preserving non-autoreactive subpopulations, which are damaged by treatments that is targeting nonspecific differentiation markers (e.g., CD19, CD38, BCMA) regardless of whether or not they are carrying an autoreactive BCR.

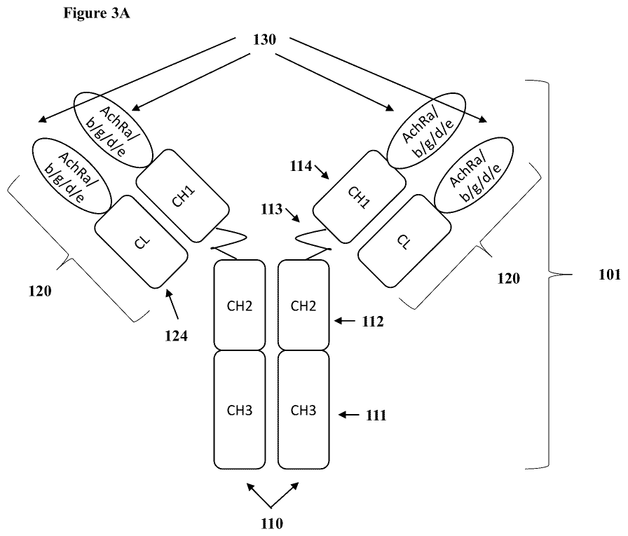

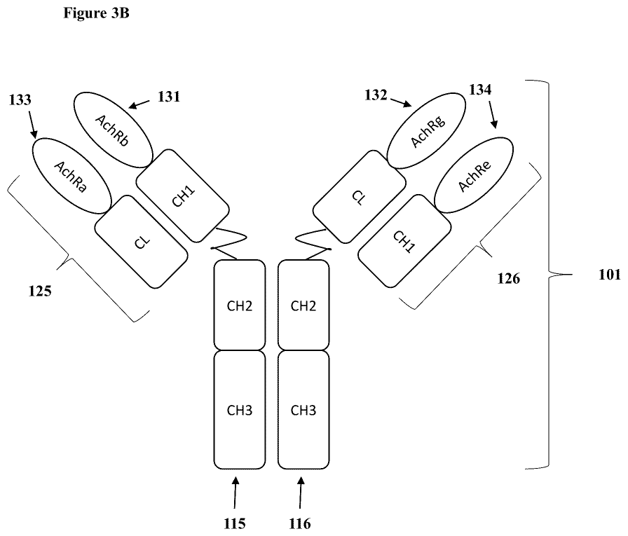

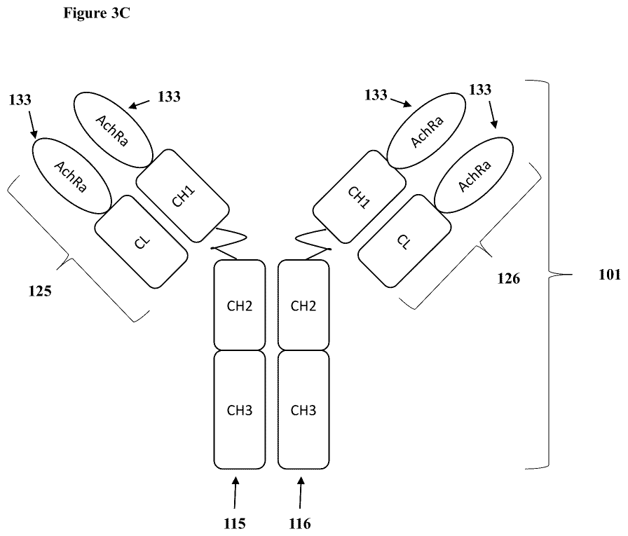

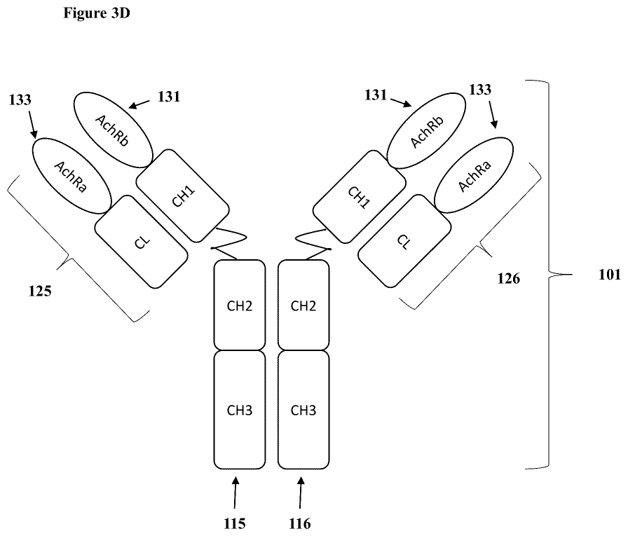

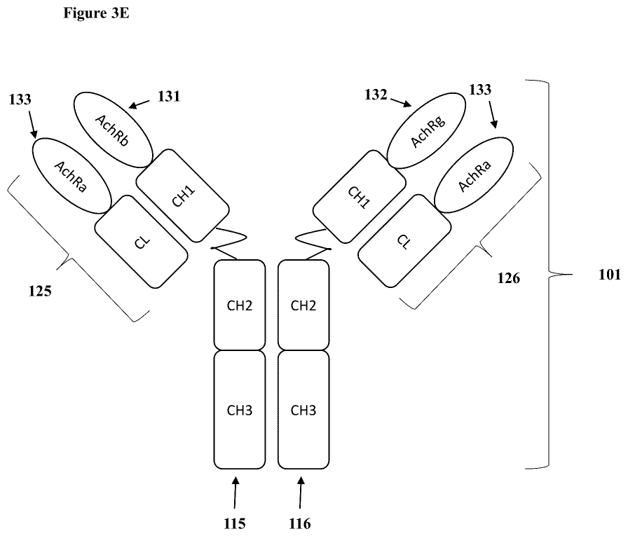

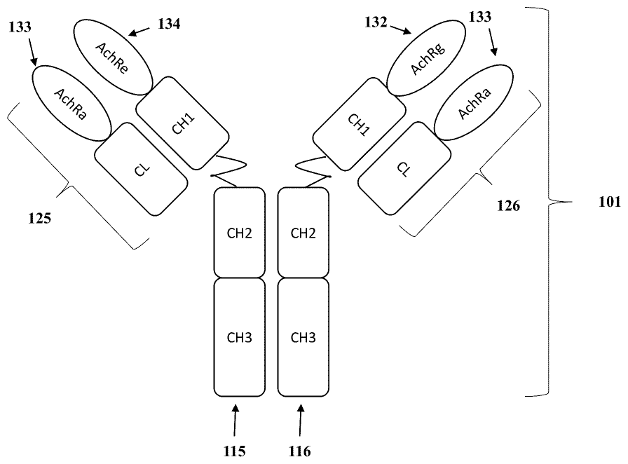

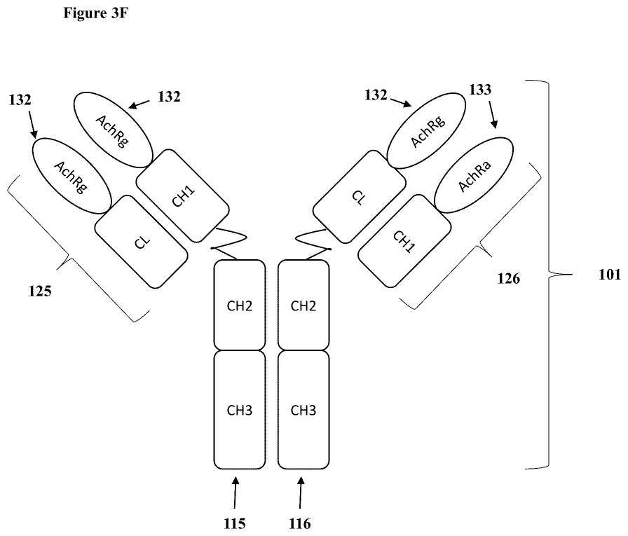

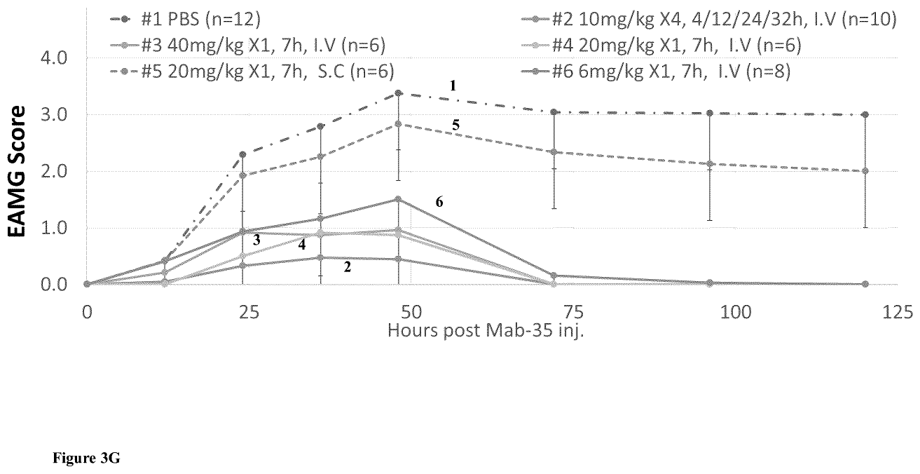

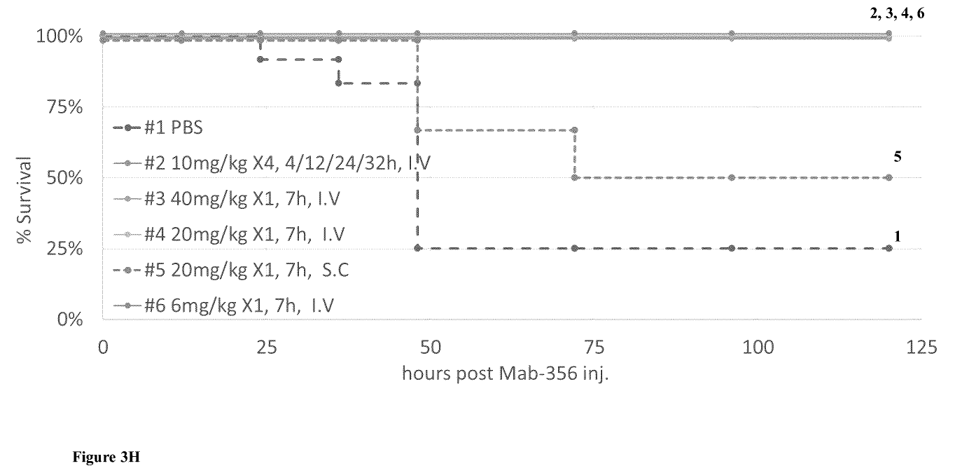

FIG. 3A shows one embodiment of the therapeutic agent of the invention. Immunoglobulin (Ig)-like protein complex 101 comprises four polypeptide chains: two heavy-chain-like polypeptides 110 and two light-chain-like polypeptides 120. Chains 110 are able to dimerize via disulfide bonds between them. Further, chains 110 may comprise any or all of CH3 domain 111, CH2 domain 112, hinge region 113 and CH1 domain 114. In this embodiment, the hinge region 113 comprises disulfide bonds and acts as the dimerization domain, though use of other dimerization domains is also possible. These domains are well known in the art and can be selected from any of human IgG1, IgG2, IgG3, IgG4, IgA, IgM, IgE, and IgD domains for example. A skilled artisan will appreciate that the Fc portion of IgG1 and IgG3 incorporated into chain 110 will allow the molecule to induce antibody dependent cellular cytotoxicity (ADCC) and complement dependent cytotoxicity (CDC). Chains 120 are able to dimerize with chains 110 via disulfide bonds found in CH1 domain 114 and CL domain 124.

Chains 110 and 120 are devoid of variable regions, unlike naturally occurring or manmade antibodies. In place of the variable region each chain has a fragment 130 from the extracellular portion of the human acetylcholine receptor. Each chain can be generated to have the same AChR subunit or different subunits. Indeed, as shown in FIG. 3B, the two heavy chains 115 and 116 can be engineered separately such that chain 115 contains, for example the beta subunit 131 and chain 116 contains the gamma subunit 132. The same is true for light chains 125 and 126, which, for example, can contain the alpha 133 and epsilon 134 subunits. Thus, the therapeutic molecule can be designed with four copies of a single subunit (FIG. 3C), two copies each of two different subunits (FIG. 3D), or one copy each of four different subunits (FIG. 3B) or any other combination thereof. Indeed, the molecule is sufficiently modular that it could be engineered with three copies of one subunit and one copy of another subunit, or two copies of one subunit and one copy of two other subunits. FIG. 3E shows embodiments where the two light chains are identical, but the two heavy chains are different. And FIG. 3F shows embodiments where the two heavy chains are identical, and the two light chains are different. Importantly, the therapeutic molecule can be engineered to comprise four of the five different AChR subunits, which as explained above can induce over 50% inhibition in at least 50% of AChR positive patients and can induce at least 40% inhibition in all tested patients. It will be understood by a skilled artisan that any chain can include any subunit, and the combinations of chains and subunit depicted in FIGS. 3A-F are meant only to be illustrative and not limiting.

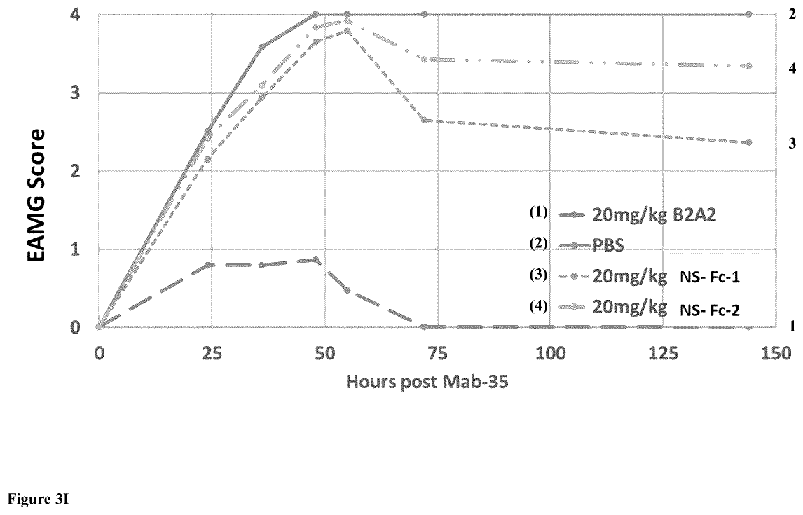

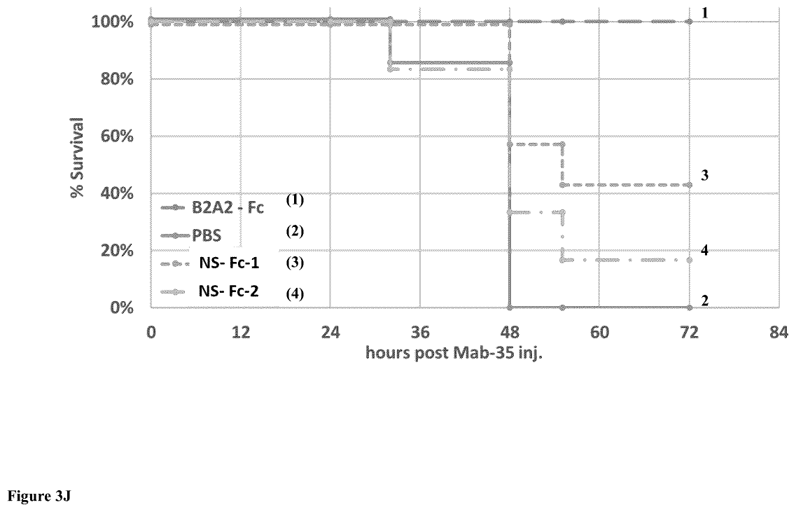

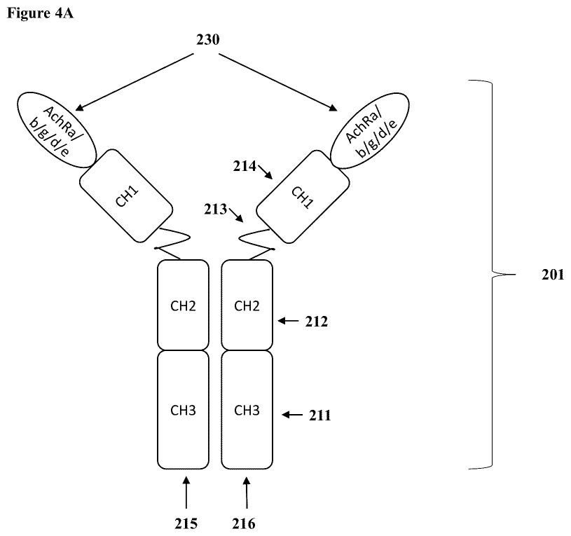

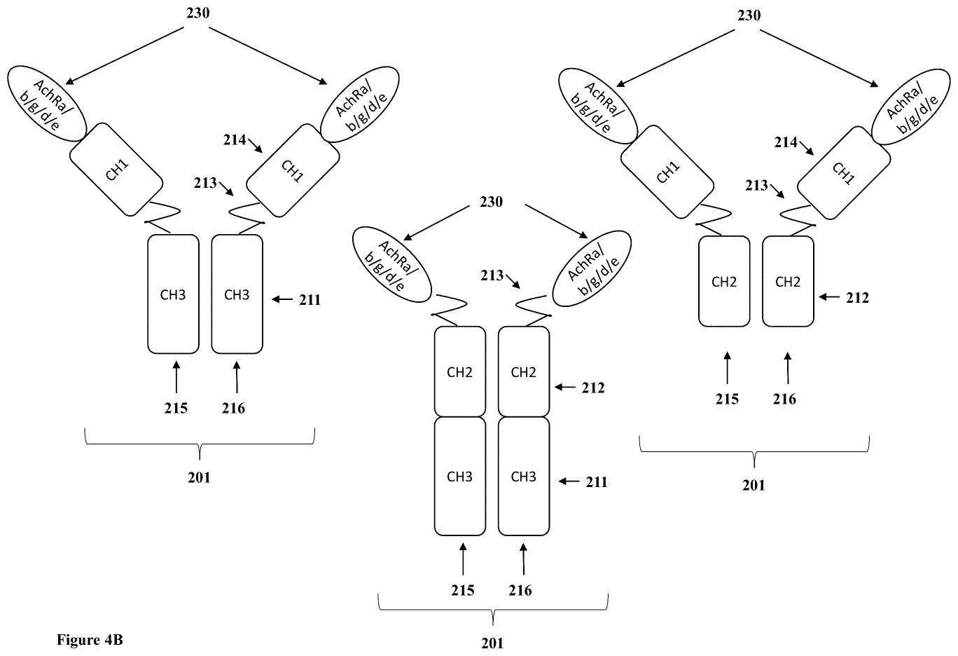

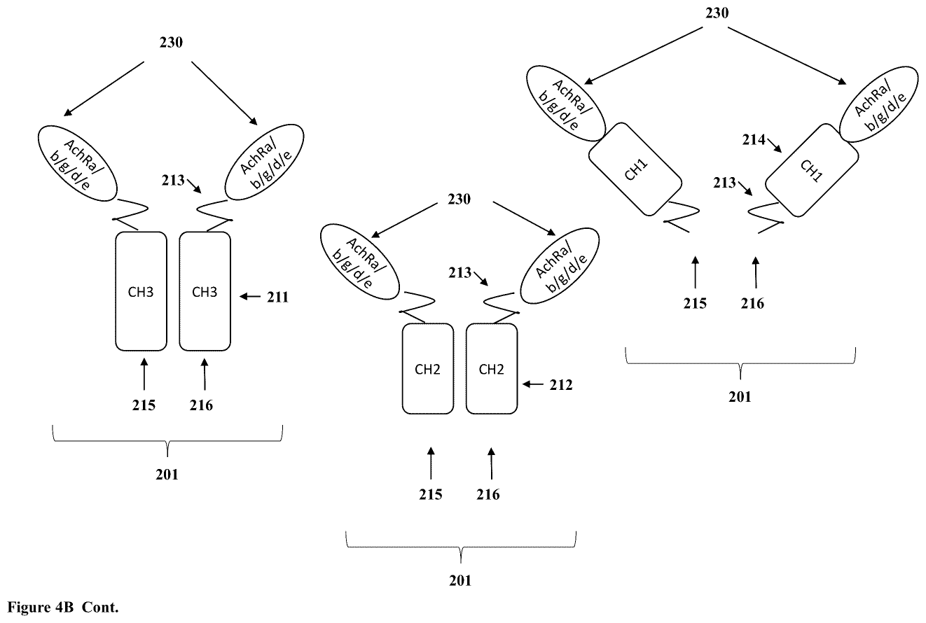

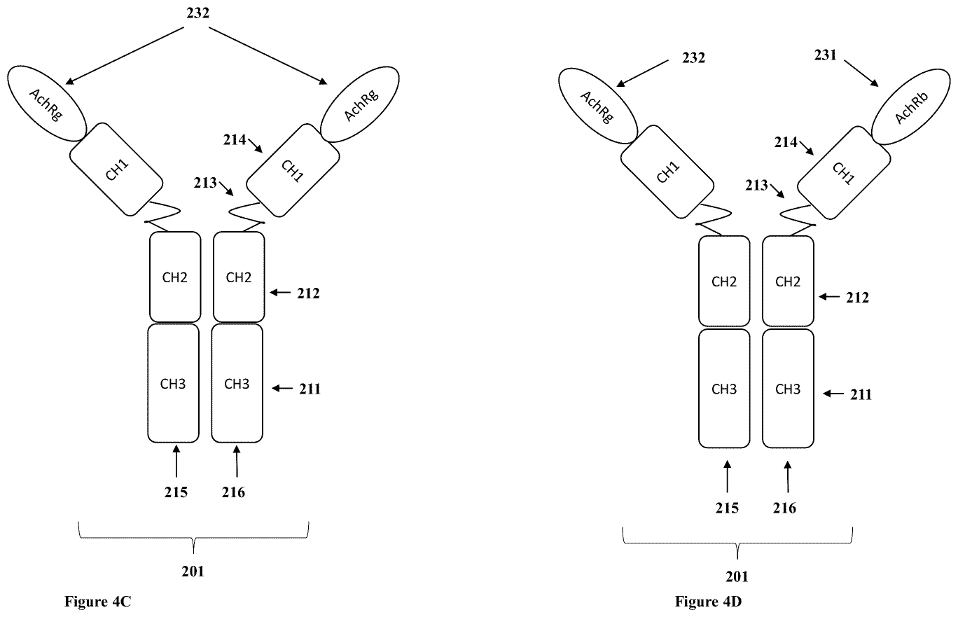

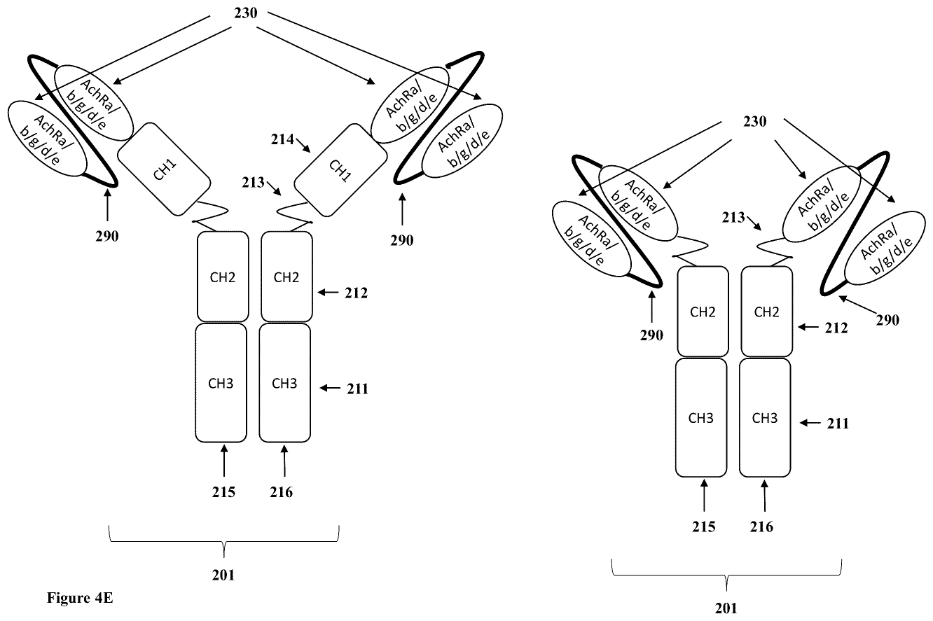

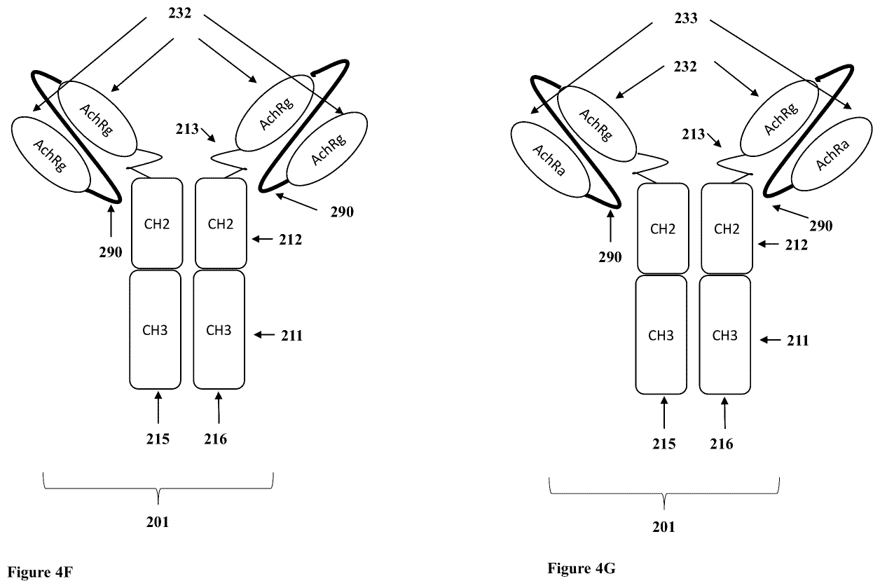

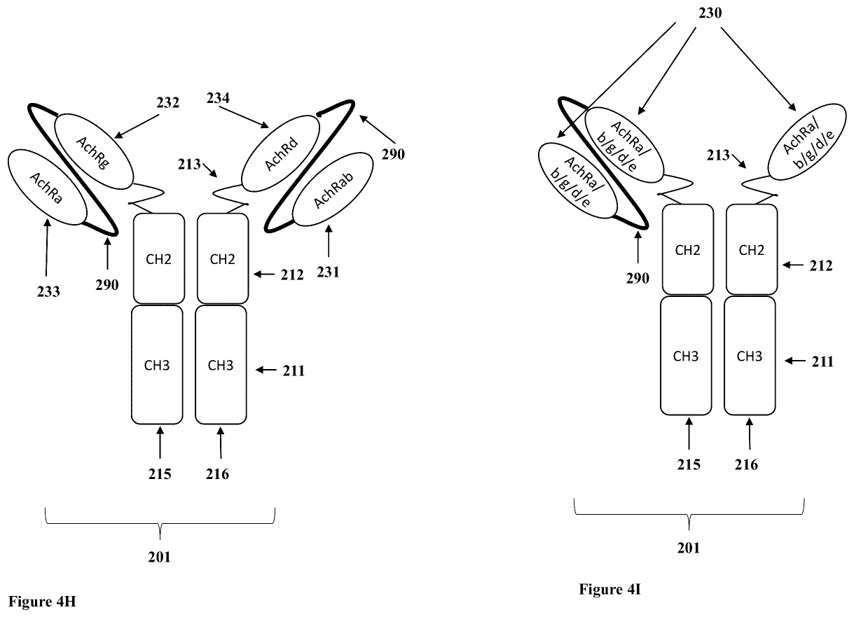

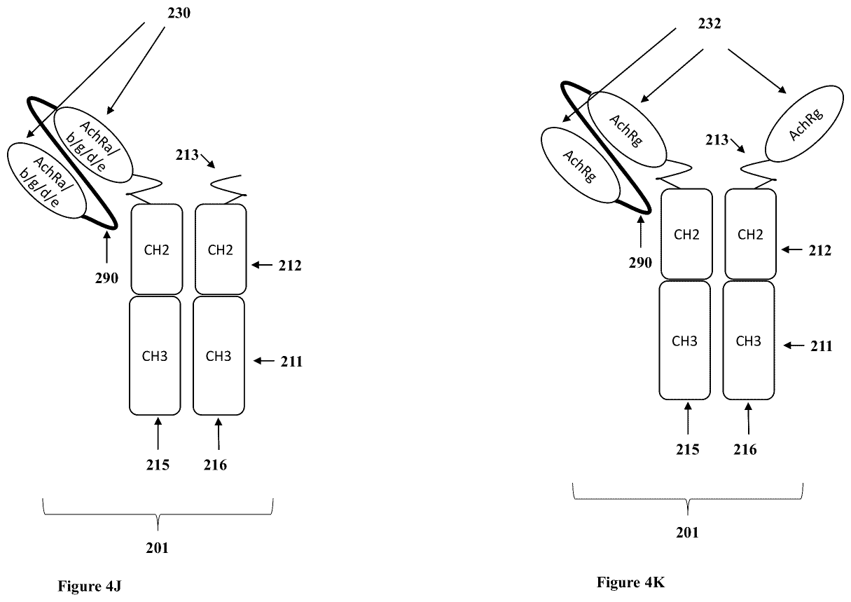

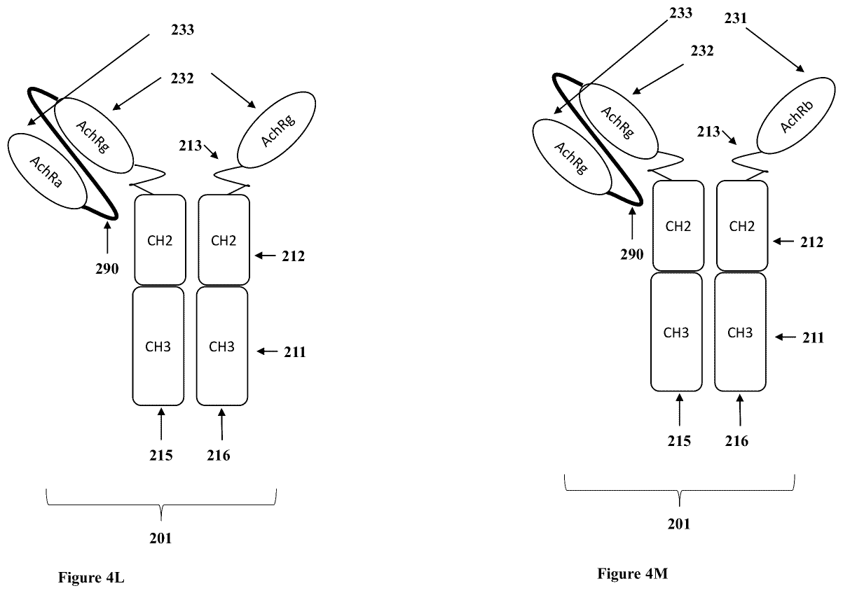

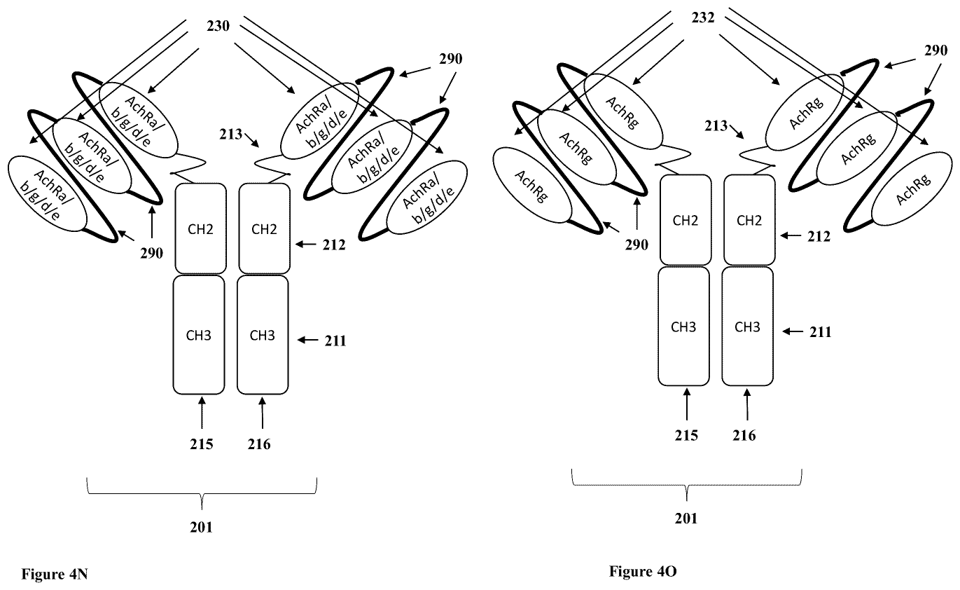

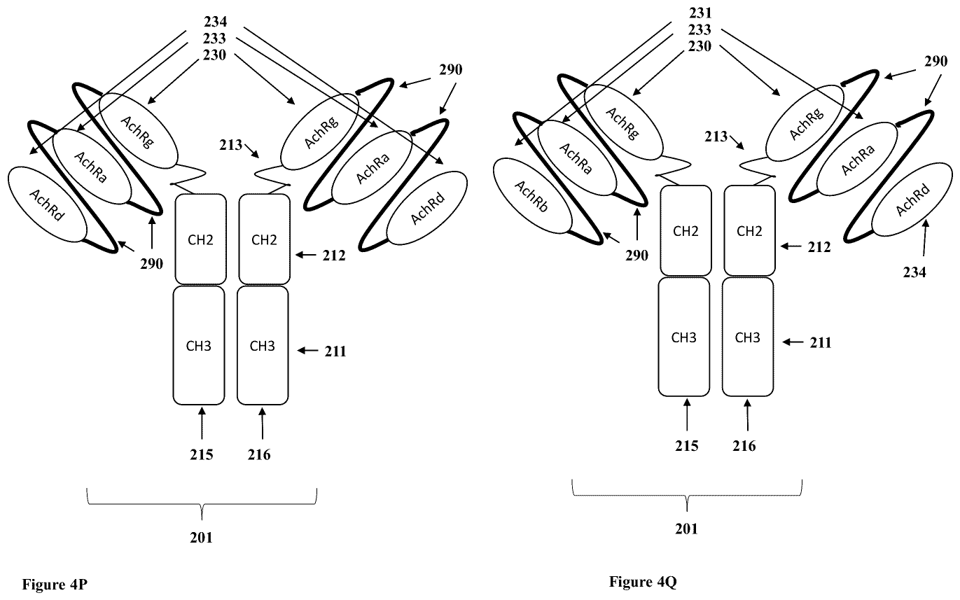

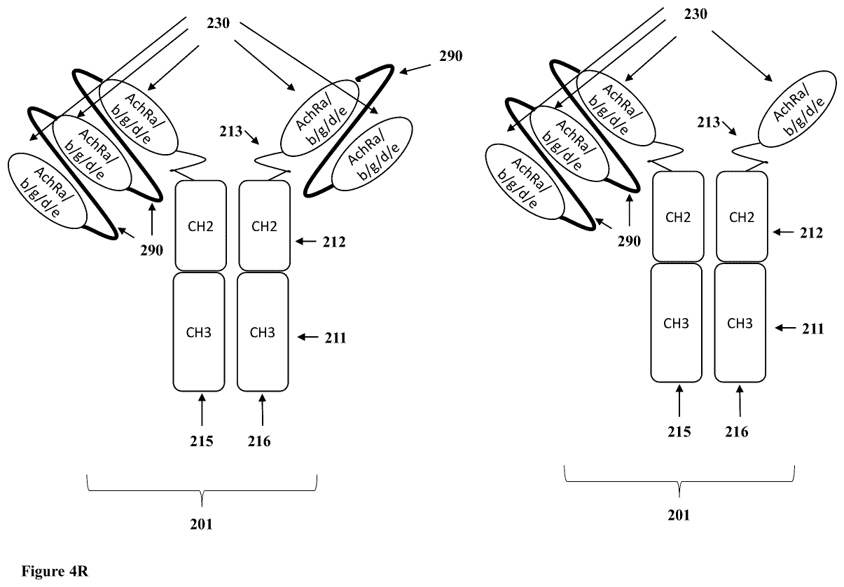

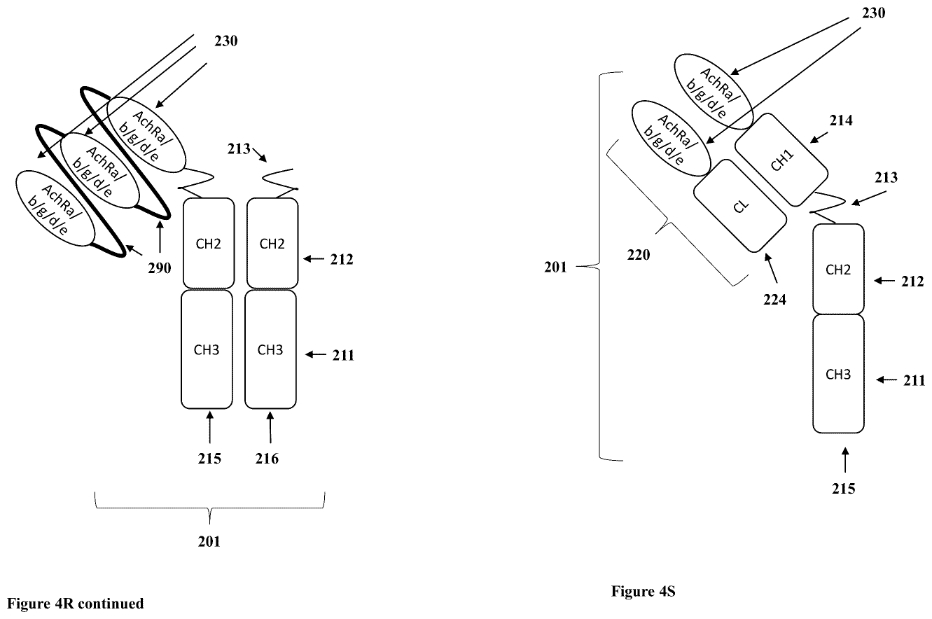

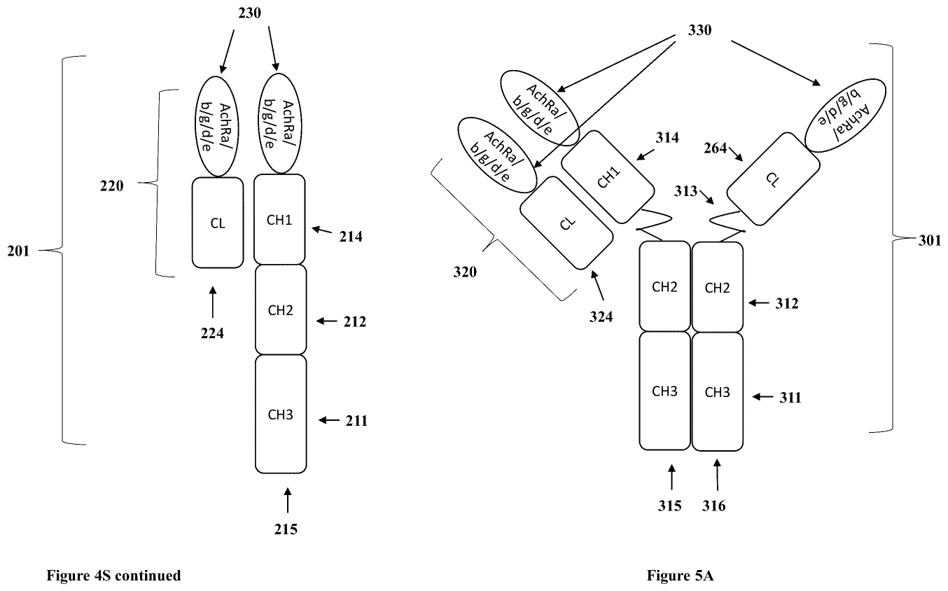

FIGS. 4A-F show some embodiments of the invention in which only two chains are combined. In FIGS. 4A-E, protein complex 201 comprises 2 polypeptide chains which specifically are two heavy chains. Heavy chains 215 and 216 can optionally include a CH2 212, CH3 211 and/or CH1 214 domain. In this embodiment, the dimerization domain is the heavy chain hinge 213 which dimerizes via disulfide bonds, although other dimerization domains are also envisioned. FIG. 4B shows the molecule without CH2 domain 212 or CH3 domain 211 or CH1 domain 214. Combinations lacking two of these domains are also envisioned (FIG. 4B). In place of the variable region each chain has a fragment 230 from the extracellular portion of the human acetylcholine receptor. Each chain can be generated to have the same AChR subunit (FIG. 4C) or different subunits (FIG. 4D). When two different subunits are employed, it is advantageous to design the molecule such that predominantly heterodimers of 215 and 216 are formed and not homodimers. The same is true of forming heterodimers of chain 115 and chain 116 in FIG. 3. There are numerous technologies known in the art for designing mutations in the CH3/CH2 domains, such as Knobs-in-Holes, DuoBodies, etc., that inhibit homodimerization and promote heterodimerization. Any such technology may be employed.

In FIG. 4E alternative configurations comprising two heavy chains are shown. Instead of containing a single fragment 230 in place of the variable region, two tandem fragments 230 are used. These fragments may be separated by optional linker 290. This configuration is similar in structure to a single chain antibody in which the heavy and light chain variable are on a single peptide and is essentially equivalent to the molecule shown in FIG. 3D. Heavy chains 215 and 216 can optionally include a CH2 212, CH3 211 and/or CH1 214 domain. In this embodiment, the dimerization domain is the heavy chain hinge 213 which dimerizes via disulfide bonds, although other dimerization domains are also envisioned. For simplicity an example containing all three CH domains is shown as is an example lacking the CH1 domain. Molecules lacking the CH2 or CH3 domain or lacking any two of these domains are also envisioned. It will be understood that fragment 230 can be from any AChR subunit. Thus, a repeat of two of the same subunits can be inserted on a single chain (FIG. 4F, two AChRg subunits 232) or two different subunits can be combined on one chain (FIG. 4G, an AChRg subunit 232 and an AChRa subunit 233). Of course, the heavy chains need not be identical as various technologies may be used to favor heterodimerization over homodimerization (FIG. 4H, an AChRg subunit 232 and an AChRa subunit 233 on one chain and an AChRd subunit 234 and AChRb subunit 231 on the other chain).

Of course, if the two heavy chains are not identical the molecule can be designed with only one of the heavy chains containing two tandem fragments 230 and with the other heavy chain containing a single fragment 230 (FIG. 4I) or no fragment (FIG. 4J). The same fragment could be included on both chains in all positions (FIG. 4K, two AChRg subunits 232 on one chain and a single AChRg subunits 232 on the other chain), included on both chains but with a different subunit in the tandem location (FIG. 4L, an AChRg subunit 232 and an AChRa subunit 233 on one chain and a single AChRg subunits 232 on the other chain), or all three fragments could be different (FIG. 4M, an AChRg subunit 232 and an AChRa subunit 233 on one chain and a single AChRb subunits 231 on the other chain).

In FIG. 4N alternative configurations comprising two heavy chains are shown in which three tandem fragments 230 are used on both chains. These fragments may be separated by optional linker 290. As with two tandem fragments, configurations wherein the fragments are the same (FIG. 40), or different (FIG. 4P) are possible. Further, the two chains need not contain the same fragments (FIG. 4Q) or even the same number of fragments (FIG. 4R). It will be understood that though FIGS. 4F-R show molecules with only CH2 212 and CH3 213 domains they can also be constructed to include the CH1 domain 214 or even only one of the domains (CH1, CH2 or CH3).

In FIG. 4S, protein complex 201 comprises 2 polypeptide chains which specifically are a heavy chain 215 and a light chain 220. In such an embodiment the dimerization domains are CH1 domain 214 and CL domain 224. Heavy chain 215 may optionally include CH3 domain 211, CH2 domain 212 and/or hinge region 213. Absence of the hinge domain is one option for eliminating homodimerization of two heavy chains 215. Alternatively, cysteine substitutions/mutations (to serine or glutamine for example) may be introduced into the hinge or one of the mutations in the CH2/CH3 regions that promote heterodimerization and inhibit homodimerization may be employed. In place of the variable region each chain has a fragment 230 from the extracellular portion of any of the human acetylcholine receptor subunits.

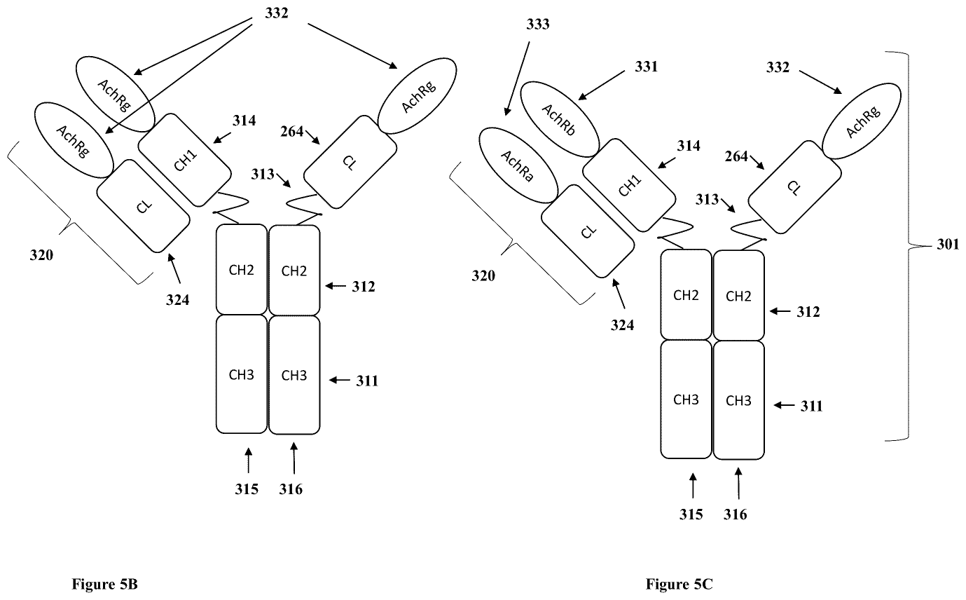

The creation of a protein complex 301, which has three chains, a heavy chain 315, a heavy chain 316 and a light chain 320 is also envisioned (FIG. 5A-D). FIG. 5A shows one possible embodiment in which heavy chain 316 comprises a CL domain 364 in place of a CH1 domain. The hereinabove described methods of ensuring a 315/316 heterodimer can be employed. Heavy chains 315 and 316 may optionally include CH3 domain 311, CH2 domain 312 and/or hinge region 313 or may employ a different dimerization domain. CL domain 324 within light chain 320 can only dimerize with CH1 domain 314 within heavy chain 315. In place of the variable region each chain has a fragment 330 from the extracellular portion of any of the human acetylcholine receptor subunits. The three chains can all contain the same fragment (for example AChRg fragment 332, FIG. 5B), all three chains can contain different fragments (for example AChRb fragment 331, AChRg fragment 332 and AChRa fragment 333, FIG. 5C), or the three chains can contain two different fragments in which one is repeated (for example AChRg fragment 332 and AChRa fragment 333, FIG. 5D). It will be understood by a skilled artisan that FIG. 5D could also have the two identical fragments as the light chain and either of the heavy chains, thus there are 3 different configurations to this embodiment.

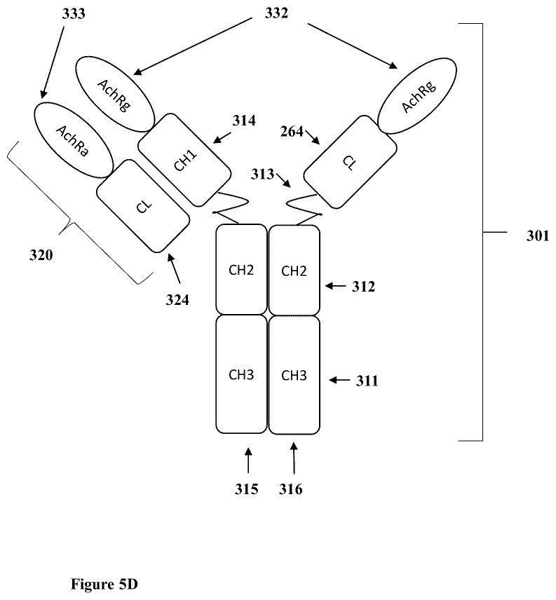

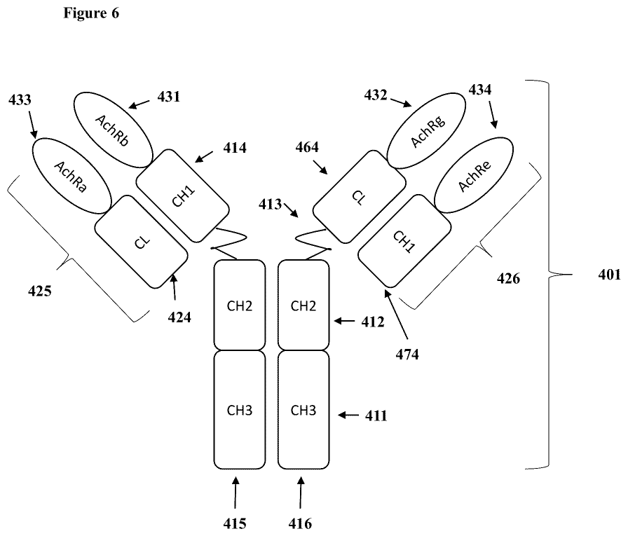

This configuration, with one of the heavy chains comprising a CL domain in place of a CH1 domain can also allow for the formation of the protein complex with four different fragments. Similar to the protein complex of FIG. 3B, protein complex 401 depicted in FIG. 6 has four different fragments on each chain. In this embodiment, the fragments AChRa 433, AChRb 431, AChRg 432 and AChRe 434 are employed but a skilled artisan will appreciate that any four fragments can be used. For the treatment of MG specifically any four of AChRα, AChRb, AChRg, AChRd, and AChRe, can be used. Embodiments are also envisioned in which different fragment from the same protein can be on different chains. In this embodiment, the second light chain 426 contains a CH1 domain 474 so that it can dimerize with the CL domain 464 in heavy chain 416. Heavy chain 415 will contain a CH1 domain 414 and light chain 425 will contain a CL domain 424. This ensures that chain 425 can dimerize only with chain 415 and chain 426 can dimerize only with chain 416. As described hereinabove, mutations in the optional CH2 domains 412 and the CH3 domains 413 can be employed to promote heterodimerization of chains 415 and 416. Hinge region 413 is used here as the dimerization domain between the two heavy chains, though any dimerization domain (other than CH1/CL) can be employed.

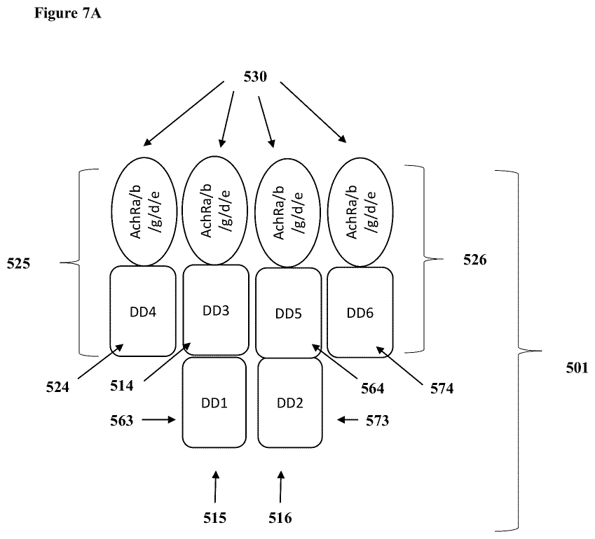

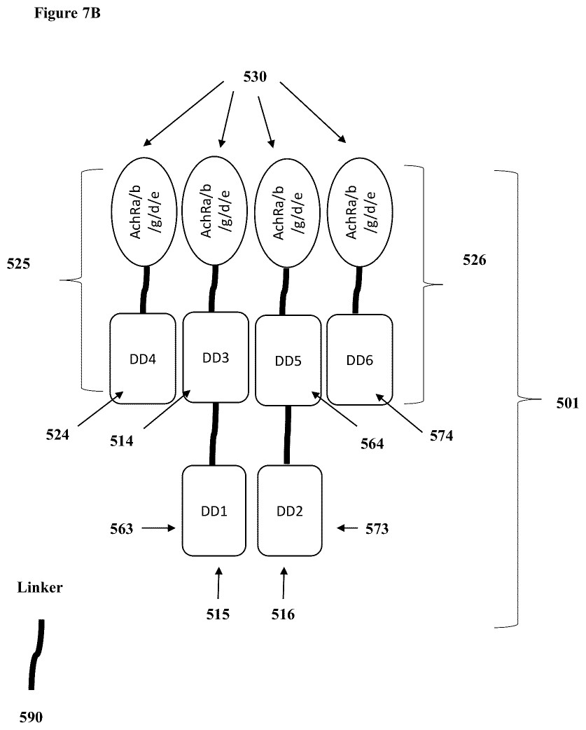

In the above-described embodiments, an immunoglobulin backbone is depicted and described, but it will be understood by a skilled artisan that by selecting other dimerization domains similar molecules. FIGS. 7A-D show a generic protein complex 501. In FIG. 7A the first chain 515 contains a first dimerization domain (DD1) 563 which can dimerize specifically with a second dimerization domain (DD2) 573 of second chain 516. Chain 515 further comprises a third dimerization domain (DD3) 514 which can dimerize specifically with a fourth dimerization domain (DD4) 524 of third chain 525. Chain 516 further comprises a fifth dimerization domain (DD5) 564 which can dimerize specifically with a sixth dimerization domain (DD6) 574 of fourth chain 526. Each of the four chains also comprises a fragment 530 of a human protein target of myasthenia gravis autoantibodies. As discussed hereinabove these targets include AChRα, AChRb, AChRg, AChRd, and AChRe. These can be all the same fragment with the same amino acid sequence, or they can be different sequences (either from the same protein or from different proteins).

FIG. 7B shows an alternative embodiment to FIG. 7A in which each distinct domain is separated by a linker. It will be understood by a skilled artisan that all of these linkers are optional, and that combination of linkers is envisioned. It will be further understood that the configurations of FIG. 7B also could employ linkers between any or all of the various domains/fragments.

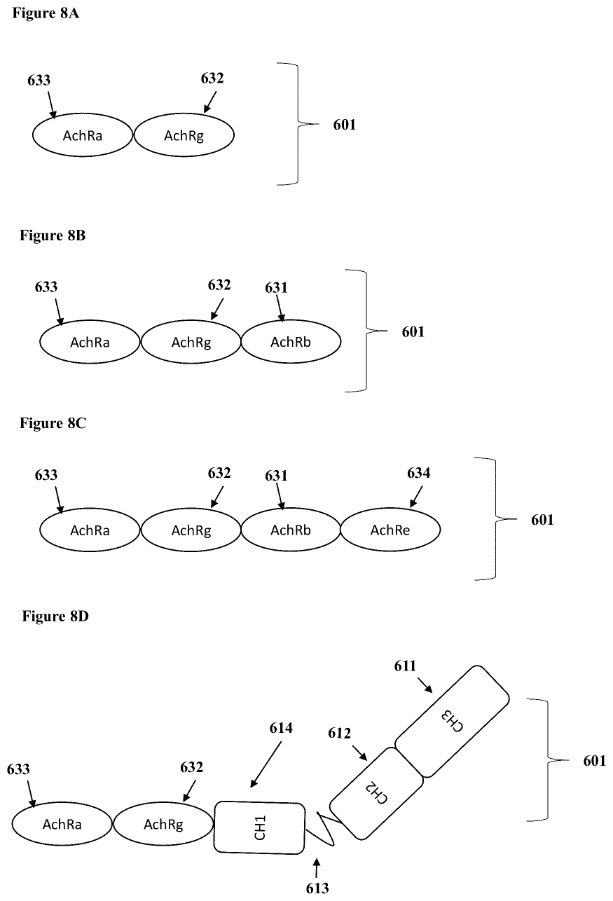

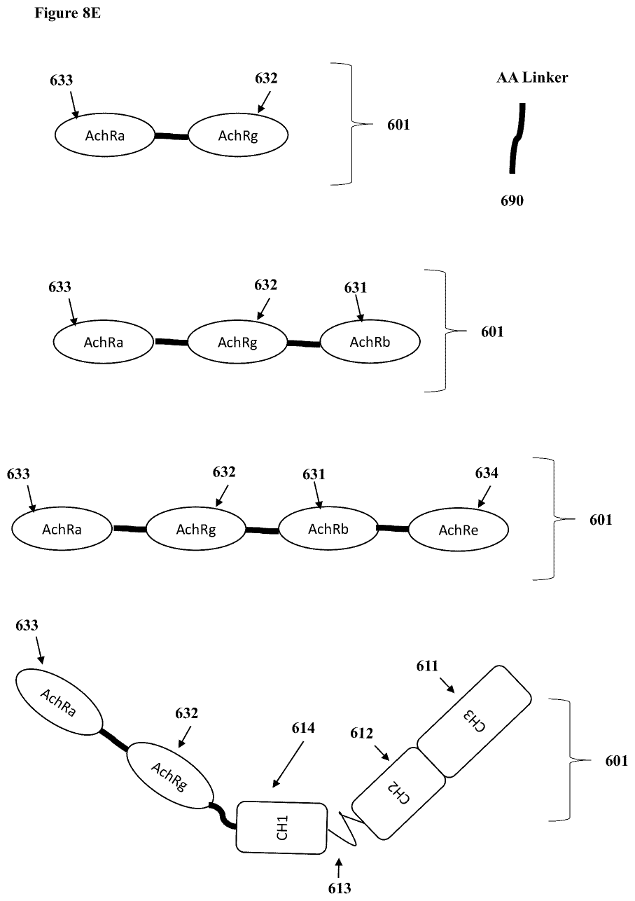

In FIG. 8A-E single chain embodiments of the invention are depicted. FIG. 8A shows a single chain fusion protein 601 containing a fragment of a first acetylcholine receptor subunit (AChRα, 633) and a fragment of a second acetylcholine receptor subunit (AChRg, 632). It will of course be understood that any permutation of two different fragments can be used. Specifically fragments from two different proteins can be used. FIGS. 8B and 8C show a similar embodiment but containing 3 and 4 fragments from different proteins respectively. As shown in FIG. 8D, the single chain can also contain a heavy chain constant region with at least a CH3 domain 611 and optionally CH1 domain 614, hinge region 613 and/or CH2 domain 612. Finally, amino acid linkers can be used to separate any of the domains of the single chain. FIG. 8E depicts embodiments, with 2, 3, or 4 fragments all separated by linkers 690 as well as an embodiment in which fragments 633 and 632 are separated by linkers 690 and a linker 690 also separates the C-terminal fragment 632 from CH1 domain 614. Although, no linkers are depicted separating the CH1 domain 614, the hinge region 613, the CH2 domain 612 and the CH3 domain 611, it will be understood by a skilled artisan that any or all of these domains could be separated by linkers. Further, it will be understood that these various linkers can all contain the same sequence or can be made of different amino acid sequences. Also, it will be understood that permutations with only 1 or all 5 fragments are envisioned.

Although not depicted in FIGS. 4-8, the acetylcholine receptor subunits may contain mutations. Such mutations may increase solubility, such as is described hereinabove. Such mutations may inhibit ligand binding, such as is described hereinabove. Such mutations may decrease aggregation, such as is described hereinabove. Any of the constructs described above and shown in FIGS. 4-8 may include such mutations in one or many of the fragments.

The various above-described molecules were generated in various forms. Loop replaced subunit ECDs (SEQ ID NO: 131-135) with increased solubility (as disclosed in Lazaridis et al., 2014, “Expression of human AChR extracellular domain mutants with improved characteristics”, Int J Biol Macromol. 2014 February; 63:210-7, hereby incorporated by reference in its entirety) were used to generate these molecules as far greater yields of protein could be produced due to the increased solubility.

### Example 4

To further test the ability to treat a large proportion of the MG population, the in vitro depletion assay described herein above is repeated in a larger cohort. The optimal combination of AChR subunits from the five ECD-loop replaced-subunits: alpha-1, beta-1, gamma, delta and epsilon is determined based on the ability to neutralize anti-AChR antibodies in at least 50% of the population. The following molecules (protein complex) of the invention: 1) α1-CL/β1-CH1-CH2-CH3 (FIG. 3D); 2) α1-CL/β1-CH1-CH2-CH3/γ-CH1-CH2-CH3 (FIG. 3E) or 3) α1-CL/β1-CH1-CH2-CH3/ε-CH1-CH2-CH3 (FIG. 3E) are then employed in the assay showing that it produces this elevated level of blocking.

### Induction and Clinical Evaluation of MG Models in rat

Passive Transfer MG Model: Female Lewis rats, were intraperitoneally or subcutaneously injected with antibodies against AChR subunits in order to induce MG and were assessed for changes in EAMG clinical score by following the method of Losen et al. Experimental Neurology 270, 2015 herein incorporated by reference in its entirety. The molecule of the invention was intravenously or subcutaneously administered at different doses and at different time points after anti-AChR-subunit antibodies administration and the ability of the molecule of the invention to reduce the titer of autoreactive antibodies, lower the MG clinical score and increase the overall survival of the treatment group compared to the control is determined. In order to examine the alpha-1 AChR subunit the molecule of the invention α1-CL/β1-CH1-CH2-CH3 (FIG. 3D) was intravenously administered at different doses and at different time points after mAb35 anti-alpha-1 monoclonal antibody administration. Rats of group #1 (n=12) received PBS as a negative control. Rats of group #2 (n−10) received four doses of 10 mg/kg of the molecule of the invention. Doses were given at 4, 12, 24 and 32 hours after antibody administration. Rats of group #3 (n=6) received 1 dose of 40 mg/kg at 7 hours after antibody administration. Rats of group #4 (n=6) received 1 dose of 20 mg/kg at 7 hours after antibody administration. Rats of group #6 (n=6) received 1 dose of 6 mg/kg at 7 hours after antibody administration. Rats of group #5 (n=6) also received 1 dose of 20 mg/kg at 7 hours after antibody administration, but this administration was subcutaneous. Regardless of dose or route of administration, the molecule of the invention consistently lowered the MG clinical score of the antibody treated rats (FIG. 3G). Further, a dose dependency was observed as 1 dose of 20 or 40 mg/kg was slightly superior to a single dose of 6 mg/kg. Interestingly, 4 doses of 10 mg/kg showed the best clinical effect. The subcutaneous administration show showing an improvement over the PBS control has significantly inferior to the I.V. administration. The overall survival of the rats was also measured (FIG. 3H). In the case of survival, the minor differences between treatments were not observable as all I.V. injections resulted in 100% survival after more than 5 days. Again, subcutaneous administration had a reduced effect.

Next, a comparison of a molecule of the invention (α1-CL/β1-CH1-CH2-CH3) to the equivalent of IVIG was tested. The mechanism by which IVIG is likely to treat MG is by reduction of overall antibody production by saturating the body with non-specific antibodies. Further, the presence of saturating levels of Fc competes for Fc receptors all over the body. To mimic this, passive MG rats were treated with either 1 dose of 20 mg/kg of the molecule by intravenous administration, or with the same dose of two different irrelevant proteins each linked to Fc. These non-specific Fcs are essentially equivalent to IVIG. As can be seen in FIG. 3I, as expected the molecule of the invention had a pronounced effect on clinical score (see also FIG. 3G). The non-specific Fc had a very mild positive effect, but one that was significantly worse than the specific molecule. Similar results were observed when mouse survival was observed (FIG. 3J), the molecule of the invention resulted in 100% survival, while the non-specific molecules produced only a modest improvement.

Analysis of affinity using Surface Plasmon Resonance (SPR): The affinity versus avidity of α1-ECD monomer and α1-CL/α1-CH1-CH2-CH3 (FIG. 3C) tetramer or α1-CH2-CH3 (FIG. 4B) dimer to mAb35 antibody is measured using SPR device. mAb35 antibody is fixed on a sensor chip as ligand. Different concentrations of α1-monomer, α1-CL/α1-CH1-CH2-CH3 tetramer (FIG. 3C) and α1-CH2-CH3 dimer (FIG. 4B) are reacted as analyte. Alternatively, α1-monomer, α1-CL/α1-CH1-CH2-CH3 tetramer (FIG. 3C) and/or α1-CH2-CH3 dimer (FIG. 4B) is fixed on a sensor chip as ligand Different concentrations of mAb35 antibody are reacted as analyte. The obtained sensor-gram is analyzed by the global fitting method using the SPR evaluation software. The ability of the above α1-ECD dimer and tetramer molecules to increase the binding strength (avidity) to mAb35 antibody compared to the α1-monomer (affinity) is determined.

### Example 5: Mutations that Decrease Subunit Aggregation

The ACHR subunits are meant to complex together to form the active receptor. However, it was observed that there is also a great deal of self-interaction resulting in high levels of aggregation when the subunits are each expressed individually. As such, mutations were generated in areas of the various subunits hypothesized to be responsible for the aggregation. In particular, free cytosines that can cause disulfide binding were abolished. Bi-interaction surfaces on the subunits were disrupted and hydrophobic surfaces were made more hydrophilic. The mutants were designed to reduce aggregation while not significantly disrupting autoantibody epitopes and antibody binding. The loop replaced subunits were used as the basis for the mutations.

Tables 2-4 provide the mutations made in the alpha, gamma and delta subunits. The beta subunit did not show substantial aggregation and so mutants were not designed for it. The molecules were expressed by transient expression in CHO cells and the proteins were purified by affinity chromatography on a nickel-column. For purification purposes a rigid linker was added to the C-terminus of each subunit followed by a His-Avi tag (HHHHHHHHPGSGLNDIFEAQKIEWHE, SEQ ID NO: 138). Proteins were visualized on both reduced and non-reduced SDS page to visualize aggregation and western blot was used to confirm the bands were indeed the aggregates of the expressed subunits. Finally, the produced protein was examined by SEC-HPLC. As all the subunits (WT and mutants) were of the same size they should elute at the same time point. Monomers of the subunit would be expected to elute last. The area under the last peak was considered to represent monomeric subunits and from this the percentage of the molecule found in the monomeric form was calculated and is provided in the Tables.

TABLE 2
Alpha subunit mutants

| | SEQ ID | | % | Yield |
|---|---|---|---|---|
| CRD# | NO: | Molecules Description | Monomer | (mg/L) |
| CRD-350 | 131 | ECD Alpha | 22.09 | 88.8 |
| CRD-412 | 72 | ECD Alpha (w/o N141) | 32.86 | 51 |
| CRD-640 | 73 | ECD Alpha (F100G) | 39.52 | 48 |
| CRD-642 | 74 | ECD Alpha (W149R) | 59.23 | 42 |
| CRD-643 | 75 | ECD Alpha (V155A) | 47.18 | 48 |
| CRD-761 | 76 | ECD Alpha (Y93F) | 42.8 | 38 |
| CRD-762 | 77 | ECD Alpha (Y93H) | 50.5 | 48 |
| CRD-763 | 78 | ECD Alpha (Y93R) | 44.72 | 16 |

TABLE 3
Gamma subunit mutants

| | SEQ ID | | % | Yield |
|---|---|---|---|---|
| CRD# | NO: | Molecules Description | Monomer | (mg/L) |
| CRD-370 | 133 | ECD Gamma | 28.77 | 54.5 |
| CRD 375 | 79 | ECD Gamma M84S | 25.82 | 48.4 |
| CRD 376 | 80 | ECD Gamma Y105E | 34.44 | 127.9 |
| CRD 378 | 81 | ECD Gamma Y117R | 32.15 | 75.7 |
| CRD 380 | 82 | ECD Gamma M84S/Y105E | 30.8 | 123.2 |
| CRD 381 | 83 | ECD Gamma M84S/ | 42.66 | 118.5 |
| | | Y105E/Y117E | | |
| CRD 382 | 84 | ECD Gamma M84S/ | 50.99 | 139.2 |
| | | Y105E/Y117R | | |
| CRD 383 | 85 | ECD Gamma Y105E/Y117E | 52.48 | 125.8 |
| CRD-510 | 86 | ECD Gamma Y105E/Y117R | 43.7 | 279.2 |

TABLE 4
Delta subunit mutants

| | SEQ ID | | % | Yield |
|---|---|---|---|---|
| CRD# | NO: | Molecules Description | Monomer | (mg/L) |
| CRD-390 | 134 | ECD Delta | 5.19 | 15.6 |
| CRD 391 | 87 | ECD Delta C108A | 19.55 | 86.2 |
| CRD 392 | 88 | ECD Delta C108I | 17.84 | 74.4 |
| CRD 396 | 89 | ECD Delta Y119R | 10.04 | 127.5 |
| CRD-574 | 90 | ECD Delta C108A, Y119R, | 35.14 | 25 |
| | | w/o N141 | | |
| CRD-638 | 91 | ECD Delta C108A, Y119R, | 24.18 | 116 |
| | | L151E | | |

All the generated mutants, other than the M84S mutation in the gamma subunit, did indeed decrease aggregation and increased the amount of monomer produced. In many cases the total yield of protein produced was also greatly increased. While the M84S mutation did not decrease aggregation it also did not substantially increase it or affect yield and this mutation has the added benefit of preventing oxidation of the exposed methionine during the shelf-life of the molecule. Some of the delta mutations produced only a modest increase in monomer, however, it was noted that dimers were also increased over much higher molecular weight aggregates. This is also a beneficial outcome and dimers, though not as desired as monomers, are superior to large aggregates.

Next, the ability of the various molecules to deplete subunit specific anti-AChR IgG antibodies from human MG sera (titer>0.5 nM) was tested. To this end avidin coated Sepharose beads were decorated with c-terminally biotinylated ECD containing molecules of the invention. MG serum samples were then incubated with or without the molecule coated beads (0.57 uM) for 1 hour at room temperature under shaking (400 RPM, orbital). Following centrifugation (3,200×g for 5 minutes), the supernatant was collected and tested for anti-AChR IgG concentration using the Euroimmun ELISA. The depletion rate was calculated as above. Depletion with the mutated ECDs was compared to depletion with the WT counterparts.

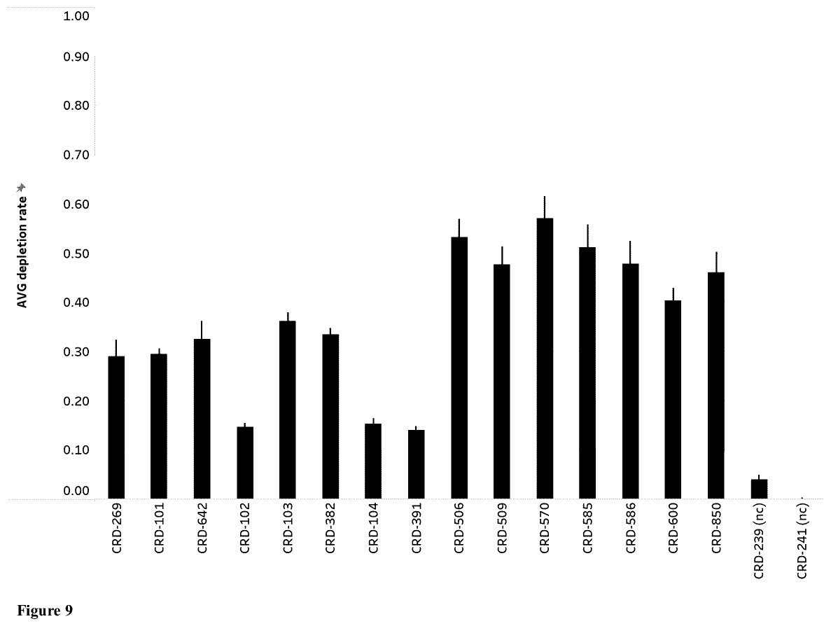

FIG. 9 summarizes the results of the depletion study by showing average depletion over many sera samples. CRD-101 containing the replacement of the cys loop of the alpha subunit in order to increase solubility was as effective as the wild-type alpha subunit fused to an Fc fragment (CRD-269, SEQ ID NO: 101). CRD-642, which contains the alpha subunit with a W149R mutation (and the loop replacement which is included in all of the following molecules), was just as effective as CRD-101. CRD-382, which contains the triple mutation M84S/Y105E/Y117R in the gamma subunit, was just as effective as the wild-type gamma subunit (CRD103). Similarly, CRD-391, containing the delta subunit with a C108A mutation, was just as effective as the wild-type delta (CRD-104).

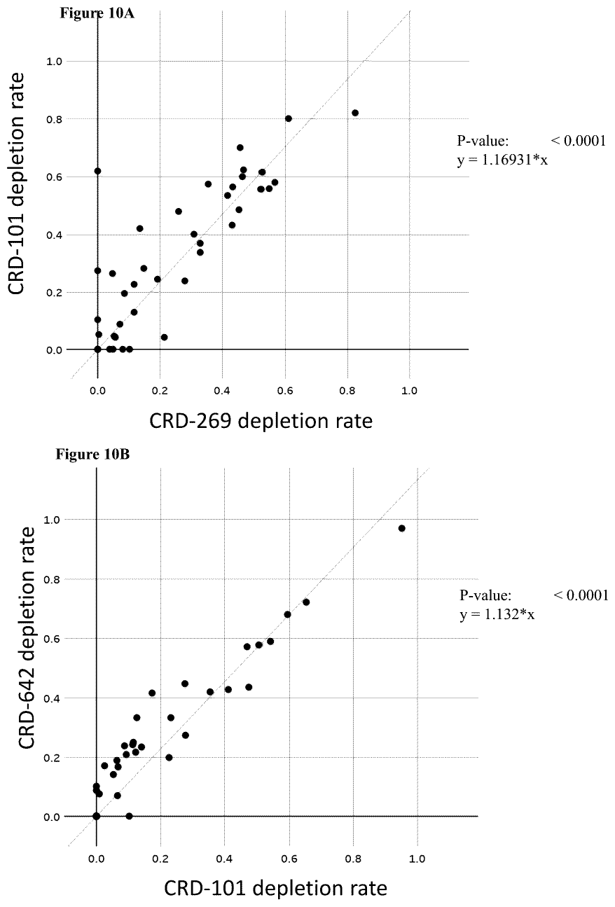

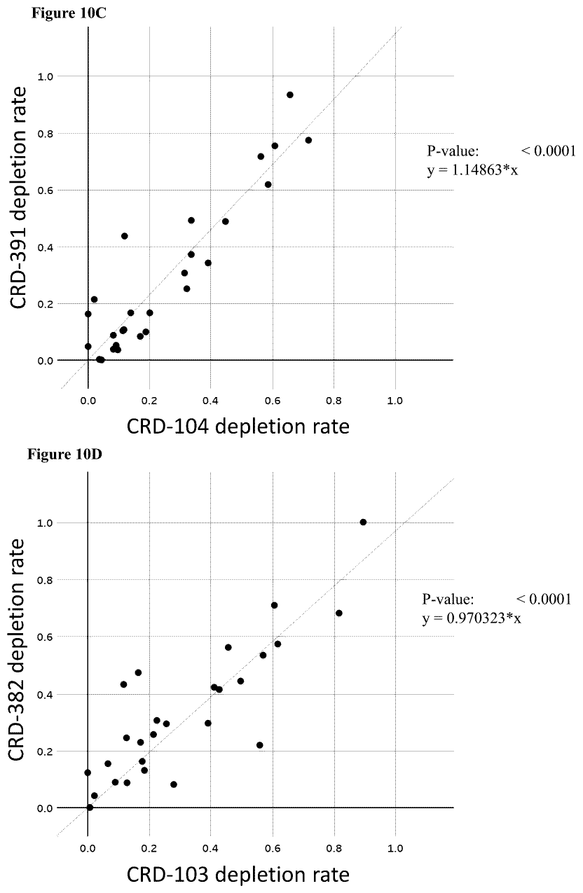

When individual sera samples were examined, it was found that many of the mutant ECDs were actually superior to their unmutated counterparts. CRD-101 (Cys loop replaced alpha subunit) was directly compared to CRD-269 (WT alpha fused to Fc) and was found to produce ˜17% more depletion which as statistically significant (p<0.0001) (FIG. 10A). The addition of the W149R mutation was found to increase depletion by ˜13% (p<0.0001) on top of the increase produced by replacement of the cys loop (FIG. 10B). The addition of the C108A mutation into the delta subunit increased depletion by ˜15% (p<0.0001) (FIG. 10C). The triple mutation in the gamma subunit, however, did not cause an increase in depletion, though it also did not decrease depletion (FIG. 10D).

### Example 6: Ability of the ECDs to Bind B Cells

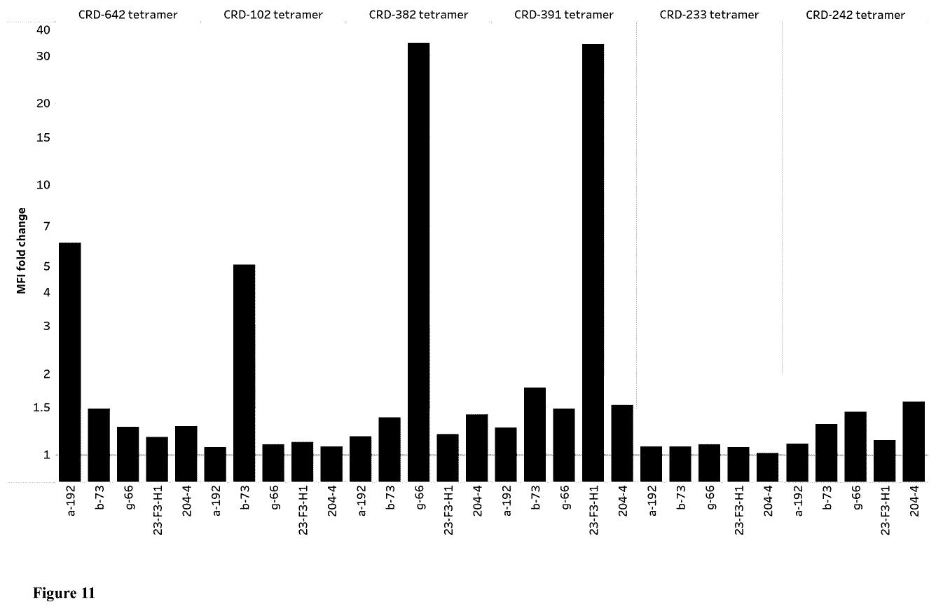

Next, the ability of the molecules of the invention to bind to B cell hybridomas expressing MG autoantibodies was tested. Fluorescently labeled streptavidin molecules were incubated with the various mutated ECD molecules C-terminally tagged with biotin. Each streptavidin molecule binds four biotin molecules, hence the resultant molecules were actually ECD tetramers. Various hybridomas expressing BCR against different AChR subunits were cultured. Four of the hybridomas expressed anti-alpha subunit antibodies (Mab35, a-192, a-195 and a-198), one expressed anti-beta antibodies (b-73), two expressed anti-gamma antibodies (g-66 and g-67) and three expressed anti-delta antibodies (43-E4-B4, 69-G11-D5-F7 and 23-F3-H1). Various hybridomas producing antibodies to irrelevant targets were used as negative controls. Following incubation of the hybridoma cells with the ECD tetramers, cells were washed (DPBS+1% FBS) twice and analyzed by flow cytometry for fluorescence on the cell surface (CytoFlex, Beckman Coulter). As can be seen in FIG. 11, each subunit tetramer specific bound to the hybridoma cells expressing antibodies against that tetramer. Non-specific binding to other hybridomas was similar to the binding to the negative control. This indicates that not only can the molecules of the invention bind MG autoantibodies in serum, but they can bind to (and kill) the B cells from which the autoantibodies originate without harming other B cells.

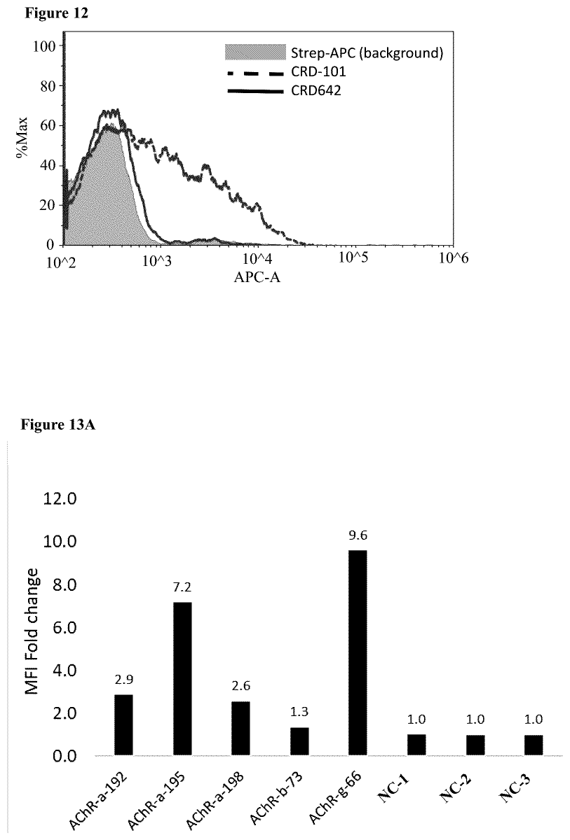

17 different hybridoma lines that produced antibodies to irrelevant targets were tested to determine non-specific/background binding. These targets included: protein targets of other autoimmune diseases (3 hybridomas), Integrin beta 3 (2 hybridomas), pathogenic agents (9 hybridomas including viruses and bacteria) and 3 human surface proteins (3 hybridomas). Surprisingly it was found that the tetramer comprising the unmutated alpha subunit (CRD-101, however, containing the cys loop substitution) bound non-specifically to at least one negative control hybridoma (AP-3, antibodies against integrin beta 3, FIG. 12). CRD-642, however, did not show this non-specific binding and thus shows unexpectedly improved specificity.

### Example 7: Molecules Comprising Combinations of ECDs

Next various combinations of the mutant extracellular domains were generated. Three different sets of combination molecules were generated using the CH2 and CH3 domains of the IgG Fc (WT IgG1 was used unless explicitly stated otherwise). In the first batch (Table 5), a combination of heavy chains one with wild-type alpha subunit and the other with mutated gamma produced a surprisingly high yield. Placing the alpha subunit and mutant gamma on the same chain but separated by a GS flexible also produced good protein yield. A combination of a wild-type alpha on one chain with a mutant delta on another chain and a combination of a chain with mutant delta and a second chain with mutant gamma both produced surprisingly high yields. Triple combinations were also generated with mutant alpha, mutant gamma and mutant delta. Both of these molecules (one with delta on its own heavy chain and one with gamma on its own heavy chain) did not show poor yield. A sortase tag (LPETG, SEQ ID NO: 139) was added to the C-terminus of some of the chains.

TABLE 5
Combination molecules

| | SEQ ID | | Yield |
|---|---|---|---|
| CRD# | NO: | Molecules Description | (mg/L) |
| CRD-506 | 92; 93 | Alpha ECD-CH2-CH3 knob-in-hole (KiH); Gamma | 524 |
| | | (Y105E/Y117R) ECD-CH2-CH3 KiH | |
| CRD-509 | 94 | Alpha ECD-GS linker-Gamma (Y105E/Y117R) ECD-CH2-CH3 | 206.8 |
| CRD-521 | 95; 96 | Alpha ECD-GS linker-Gamma (Y105E/Y117R) ECD-CH2-CH3 | 90 |
| | | KiH; Alpha ECD-GS linker-Delta (C108A) ECD-CH2-CH3 KiH | |
| CRD-570 | 97; 98 | Alpha (w/o N141) ECD-GS linker-Gamma | 76.5 |
| | | (M84S/Y105E/Y117R) ECD-CH2-CH3 KiH; Delta (C108A, w/o | |
| | | N141) ECD-CH2-CH3 KiH | |
| CRD-571 | 99; 100 | Alpha (w/o N141) ECD-GS linker-Delta (C108A, w/o N141) | 138.5 |
| | | ECD-CH2-CH3 KiH; Gamma (M84S/Y105E/Y117R) ECD-CH2- | |
| | | CH3 KiH | |
| CRD-585 | 92; 102 | Alpha ECD-CH2-CH3 KiH; Delta (C108A) ECD-CH2-CH3 KiH | 338.5 |
| CRD-586 | 103; 100 | Delta (C108A) ECD-CH2-CH3 KiH; Gamma | 368.5 |
| | | (M84S/Y105E/Y117R) ECD-CH2-CH3 KiH | |

In the second batch (Table 6) further combinations were tested. Combinations of two different heavy chains (via knob-in-holes modifications) and combinations on a single chain via linkers all produced sufficient yields. The final batch of molecules tested (Table 7) tested various other linkers (longer flexible linkers and rigid linkers) as well as changes in the Fc region such as that decrease cytotoxicity. Again, sufficient yields were produced. Thus, all the produced molecules are viable as therapeutic agents.

TABLE 6
Further combination molecules

| | SEQ ID | | Yield |
|---|---|---|---|
| CRD# | NO: | Molecules Description | (mg/L) |
| CRD-509 | 94 | Alpha ECD-GS linker-Gamma (Y105E/Y117R) ECD-CH2-CH3 | 93 |
| CRD-600 | 104 | Alpha ECD-GS linker-Gamma (M84S/Y105E/Y117R) ECD-CH2- | 100.5 |
| | | CH3 | |
| CRD-604 | 105; 130 | Alpha ECD-GS linker-Gamma (M84S/Y105E/Y117R) ECD-CH2- | 57 |
| | | CH3 KiH; Alpha ECD-GS linker-Delta (C108A) ECD-CH2-CH3 KiH | |
| CRD-615 | 105; 106 | Alpha ECD-GS linker-Gamma (M84S/Y105E/Y117R) ECD-CH2- | 79.5 |
| | | CH3 KiH; Alpha ECD-GS linker-Delta (C108A/Y119R) ECD-CH2- | |
| | | CH3 KiH | |
| CRD-616 | 105; 107 | Alpha ECD-GS linker-Gamma (M84S/Y105E/Y117R) ECD-CH2- | 49.5 |
| | | CH3 KiH; Alpha ECD-GS linker-Delta (C108A/L151E) ECD-CH2- | |
| | | CH3 KIH | |
| CRD-624 | 108 | Gamma (M84S/Y105E/Y117R) ECD-GS linker-Alpha ECD-GS | 52.5 |
| | | linker-Delta (C108A) ECD-CH2-CH3 | |
| CRD-625 | 109 | Gamma (M84S/Y105E/Y117R) ECD-GS linker-Alpha ECD-GS | 49.5 |
| | | linker-Gamma (M84S/Y105E/Y117R) ECD-CH2-CH3 | |
| CRD-766 | 110 | Alpha (Y93H) ECD-GS linker-Gamma (M84S/Y105E/Y117R) | 30 |
| | | ECD-CH2-CH3 | |
| CRD-767 | 111 | Alpha (W149R/Y93H) ECD-GS linker-Gamma | 67.5 |
| | | (M84S/Y105E/Y117R) ECD-CH2-CH3 | |
| CRD-768 | 112 | Alpha ECD-GS linker-Gamma (M84S/Y105E/Y117R) ECD-CH2- | 27.5 |
| | | CH3 (lgG4) | |
| CRD-769 | 113 | Alpha ECD-GS linker-Gamma (M84S/Y105E/Y117R) ECD-CH2- | 32.5 |
| | | CH3 (LALA) | |
| CRD-770 | 103/102 | Delta (C108A) ECD-CH2-CH3 KiH; Delta (C108A) ECD-CH2-CH3 | 616 |
| | | KIH | |
| CRD-850 | 114 | Alpha (W149R) ECD-GS linker-Gamma (M84S/Y105E/Y117R) | 230.8 |
| | | ECD-CH2-CH3 | |
| CRD-859 | 115 | Gamma (M84S/Y105E/Y117R) ECD-GS linker-Alpha (W149R) | 88.1 |
| | | ECD-GS linker-Gamma (M84S/Y105E/Y117R) ECD-CH2-CH3 | |
| CRD-860 | 116 | Gamma (M84S/Y105E/Y117R) ECD-GS linker-Alpha ECD-GS | 47 |
| | | linker-Gamma (M84S/Y105E/Y117R) ECD-CH2-CH3 KiH | |

TABLE 7
Combination molecules with altered linkers or Fc modifications

| | SEQ ID | | Yield |
|---|---|---|---|
| CRD# | NO: | Molecules Description | (mg/L) |
| CRD-850 | 114 | Alpha (W149R) ECD-(GGGGS)6-Gamma (M84S/Y105E/Y117R) | 170 |
| | | ECD-CH2-CH3 | |
| CRD-509 | 94 | Alpha ECD-(GGGGS)6-Gamma (Y105E/Y117R) ECD-CH2-CH3 | 112.5 |
| CRD-900 | 117 | Alpha (W149R) ECD-(GGGGS)6, Gamma (M84S/Y105E/Y117R) | 185 |
| | | ECD-CH2-CH3 (LALA) | |
| CRD-901 | 118 | Alpha (W149R) ECD-(GGGS)6-Gamma (M84S/Y105E/Y117R)- | 202.5 |
| | | (GGGGS)3-CH2-CH3 (LALA) | |
| CRD-902 | 119 | Alpha (W149R) ECD-(GGGS)6-GS-Gamma | 197.5 |
| | | (M84S/Y105E/Y117R) ECD-(GGGGS)3-CH2-CH3 (LALA) | |
| CRD-903 | 120 | Alpha (W149R) ECD-(GGGS)5-GS-Gamma | 172.5 |
| | | (M84S/Y105E/Y117R) ECD-(GGGGS)3-CH2-CH3 (LALA) | |
| CRD-904 | 121 | Alpha (W149R) ECD-EAAAKEAAAKGGS-Gamma | 197.5 |
| | | (M84S/Y105E/Y117R) ECD-(GGGGS)3-CH2-CH3 (LALA) | |
| CRD-905 | 122 | Alpha ECD-EAAAKEAAAKGGS-Gamma (M84S/Y105E/Y117R) | 98 |
| | | ECD-(GGGS)3-CH2-CH3 (LALA) | |
| CRD-906 | 123 | Alpha ECD-EAAAKEAAAKGGS-Gamma ECD-(GGGS)3-CH2-CH3 | 42 |
| | | (LALA) | |
| CRD-913 | 124 | CH2-CH3 (LALA)-(GGGS)3-Gamma (M84S/Y105E/Y117R) ECD- | 88 |
| | | (GGGGS)6-Alpha (W149R) ECD | |
| CRD-914 | 125 | CH2-CH3 (LALA)-(GGGS)3-Alpha (W149R) ECD-(GGGGS)6- | 90 |
| | | Gamma (M84S/Y105E/Y117R) ECD | |
| CRD-915 | 126 | CH2CH3 (LALA), (GGGS)3, Gamma (M84S/Y105E/Y117R), | 74 |
| | | (GGGS)5, Alpha (W149R) | |
| CRD-927 | 127 | Alpha ECD-(GGGGS)6-Gamma (M84S/Y105E/Y117R) ECD- | 84 |
| | | (GGGGS)3-CH2-CH3 (LALA/N297A) | |
| CRD-928 | 128 | Alpha ECD-(GGGGS)6-Gamma (M84S/Y105E/Y117R) ECD- | 74 |
| | | (GGGGS)3-CH2-CH3 (LALA/N297Q) | |
| CRD-929 | 129 | Alpha ECD-(GGGGS)6-Gamma (M84S/Y105E/Y117R) ECD- | 90 |
| | | (GGGGS)3-CH2-CH3 (LALA) | |

The molecules produced were tested for their depletion ability in the same assay as was performed on the ECDs alone. The double and triple subunit molecules appeared to produce close to an additive effect, indicating that all the subunits present are still binding their target autoantibodies when included in the molecule (FIG. 9).

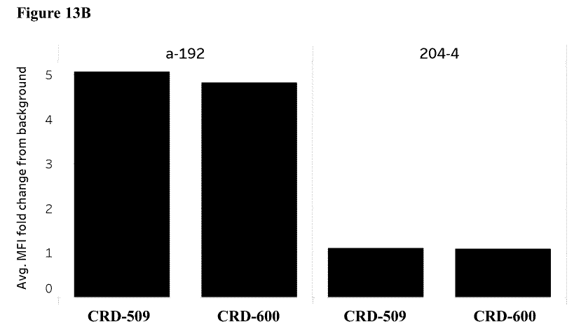

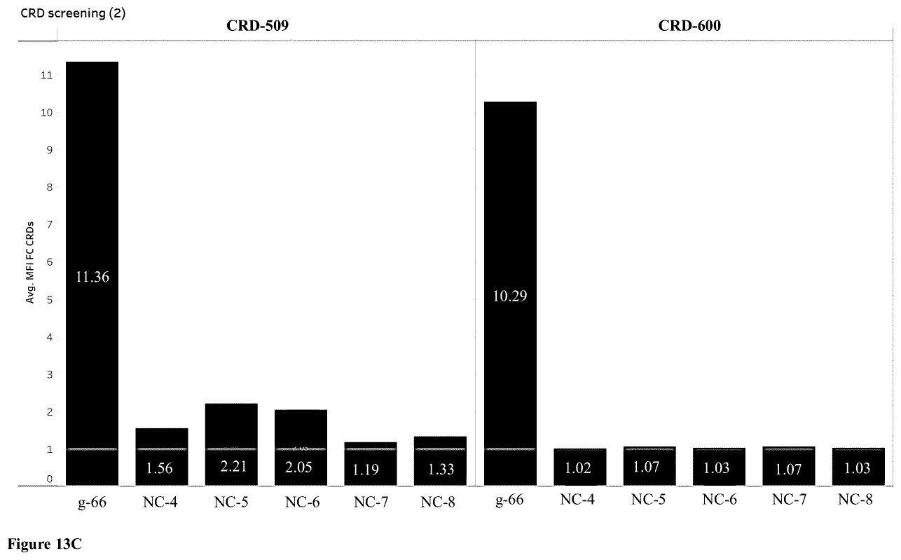

The hybridoma binding assay was also performed using the combination molecules. In this assay, molecule binding on the hybridomas was detected by FACS using a PE labeled anti-human FC polyclonal antibody. CRD-506, comprising one copy of the cys loop replaced alpha subunit and one copy of a mutated gamma subunit (also loop replaced), was incubated with three anti-alpha hybridoma lines, one anti-beta line, one anti-gamma line and three negative control lines producing antibodies to irrelevant proteins. As can be seen in FIG. 13A, the combination molecule strongly bound to all of the alpha and gamma hybridomas but not to the beta or negative control hybridomas. This demonstrates the specificity and functionality of the combination molecules.

Two other alpha-gamma molecules were also tested. CRD-509 comprises two heavy chains each with a tandem alpha and gamma subunit separated by a linker. The gamma subunit is double mutated. CRD-600 is a similar molecule, but with a triple mutation. Both molecules bound only to anti-alpha hybridoma cells and not a negative control hybridoma (FIG. 13B). Similarly, both molecules bound to anti-gamma hybridomas (FIG. 13C). Interestingly, 5 different negative control hybridoma lines were tested and while both molecules showed very little non-specific binding the triple mutant showed less than the double indicating an unexpected superiority. These results reinforce that combination of the ECDs does not abrogate their ability to bind, even when the different ECDs are on the same chain.

Several of the combined molecules were tested against a heavy chain homodimer containing only the wild-type alpha subunit (CRD-269). Unexpectedly, these molecules, although only having one copy of the alpha subunit actually were superior binders to the anti-alpha hybridoma (FIG. 14). This may be due to the replacement of the cys loop in the alpha subunit.

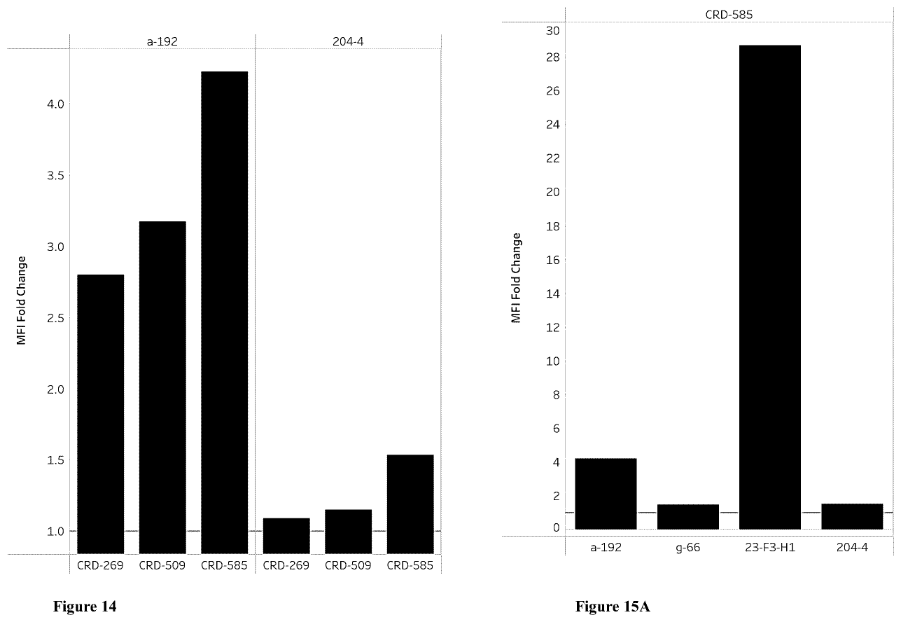

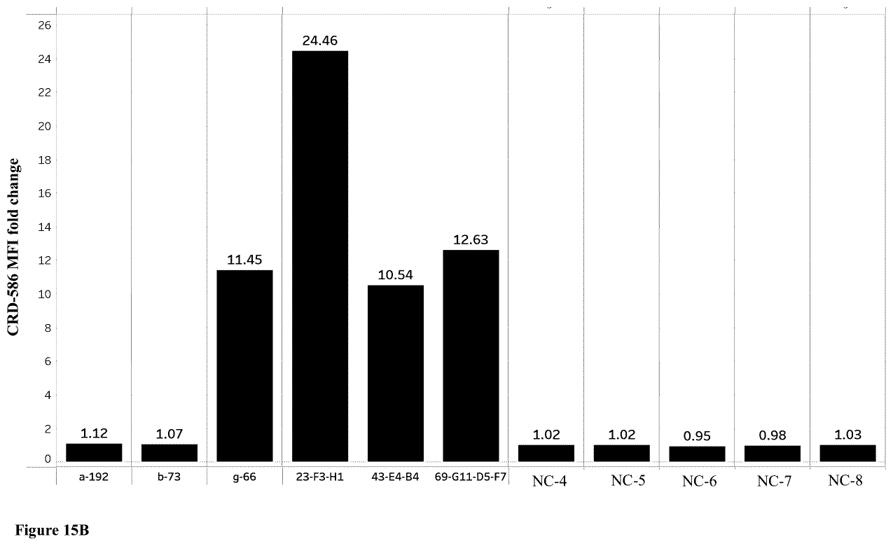

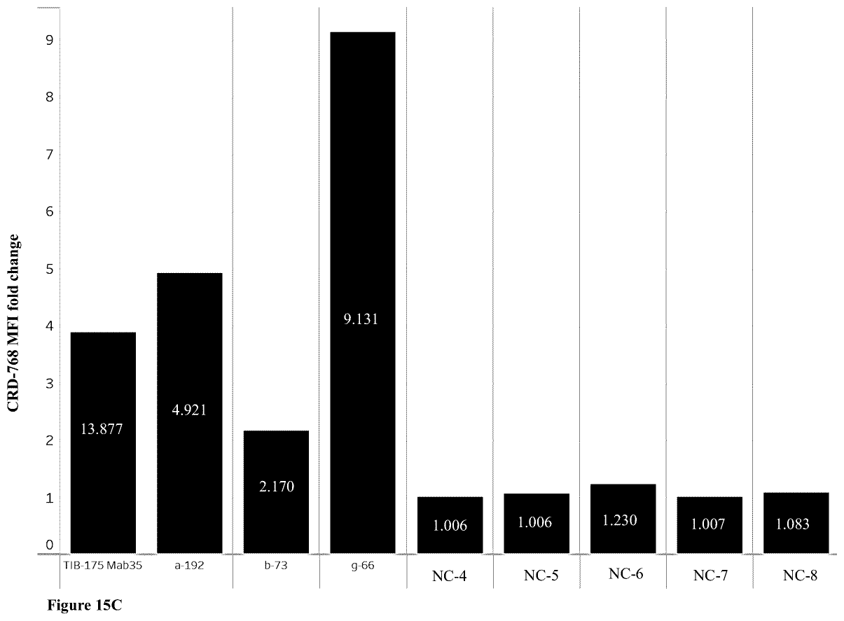

Testing of an alpha-delta double molecule (CRD-585) showed specific binding only to anti-alpha and anti-delta hybridomas (FIG. 15A). Similarly, a gamma-delta double molecule (CRD-586) showed specific binding only to anti-gamma and anti-delta hybridomas (FIG. 15B). Inclusion of an IgG4 Fc instead of an IgG1 did not negatively impact binding (FIG. 15C). Similarly, mutations in the IgG1 Fe that reduced effector function do not negatively impact binding.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A composition, comprising a protein complex comprising:
a. a first polypeptide chain comprising a fragment of a first human acetylcholine receptor subunit or an analog or derivative thereof and a first dimerization domain, and
b. a second polypeptide chain comprising a fragment of a second human acetylcholine receptor subunit or an analog or derivative thereof and a second dimerization domain,
and wherein said first and second subunits are different subunits, said analog or derivative thereof comprises at least 85% identity to said human protein and said first and second dimerization domains are configured to dimerize with each other.

2. The composition of claim 1, wherein said fragment is a fragment of an extracellular domain of said acetylcholine receptor subunit and said first and second acetylcholine receptor subunits are selected from acetylcholine receptor subunit alpha (ACHRA), acetylcholine receptor subunit beta (ACHRB), acetylcholine receptor subunit gamma (ACHRG), acetylcholine receptor subunit delta (ACHRD) and acetylcholine receptor subunit epsilon (ACHRE).

3. (canceled)

4. (canceled)

5. The composition of claim 1, wherein at least one of:
a. said dimerizing comprises forming a covalent bond between said first dimerization domain and said second dimerization domain;
b. said protein complex comprises an immunoglobulin scaffold;
c. said first dimerization domain comprises a first hinge domain of a heavy chain of an immunoglobulin and said second dimerization domain comprises a second hinge domain of a heavy chain and said first and said second dimerization domains dimerizes by a disulfide bond;
d. said first and second dimerization domains each comprise a domain selected from a CH1 domain of a heavy chain of an immunoglobulin and a CL domain of a light chain of an immunoglobulin and dimerize by a disulfide bond and wherein said first and second dimerization domains do not both comprise said CH1 domain or said CL domain;
e. said first polypeptide chain, said second polypeptide chain or both further comprise an Fc region of a human antibody heavy chain; and
f. said fragment and said dimerization domain of said first, second or both polypeptide chains are separated by a linker.

6. (canceled)

7. (canceled)

8. (canceled)

9. (canceled)

10. (canceled)

11. The composition of claim 1, wherein at least one of:
a. said first polypeptide chain comprises a first CH3 domain of a heavy chain of an immunoglobulin, a first CH2 domain of a heavy chain of an immunoglobulin or both and said second polypeptide chain comprises a second CH3 domain of a heavy chain of an immunoglobulin, a second CH2 domain of a heavy chain of an immunoglobulin or both;
b. said first polypeptide chain comprises a first CH3 domain of a heavy chain of an immunoglobulin, a first CH2 domain of a heavy chain of an immunoglobulin or both comprising at least a first mutation and said second polypeptide chain comprises a second CH3 domain of a heavy chain of an immunoglobulin, a second CH2 domain of a heavy chain of an immunoglobulin or both comprising a second mutation and wherein said mutations permit heterodimerization of said first and second polypeptide chains and inhibit homodimerization of said first polypeptide chain and homodimerization of said second polypeptide chain, optionally wherein said mutations are selected from mutations provided in Table 1;
c. said Fc is from an I2G2 or I2G4 or comprises at least one mutation that reduces effector function;
d. said Fc region of said first, second or both polypeptide chains is separated from said fragment or said dimerization domain by a linker; and
e. said dimerization domain of said first, second or both polypeptide chains is C-terminal to said fragment or N-terminal to said fragment.

12. (canceled)

13. (canceled)

14. (canceled)

15. (canceled)

16. (canceled)

17. (canceled)

18. (canceled)

19. (canceled)

20. (canceled)

21. (canceled)

22. The composition of claim 1, wherein said first polypeptide chain further comprises a fragment of said second human acetylcholine receptor subunit or an analog or derivative thereof and said second polypeptide chain further comprises a fragment of said first human acetylcholine receptor subunit or an analog or derivative thereof.

23. The composition of claim 22, wherein
a. said first polypeptide chain, said second polypeptide chain or both further comprises a fragment of a third human acetylcholine receptor subunit or an analog or derivative thereof and optionally a fragment of a fourth human acetylcholine receptor subunit or an analog or derivative thereof; and
b. said fragments are separate by an amino acid linker, optionally wherein said linker is a flexible GS linker or wherein said linker is a rigid linker.

24. (canceled)

25. (canceled)

26. (canceled)

27. The composition of claim 1, wherein said complex comprises at least one amino acid sequence selected from SEQ ID NO: 64 to 69 or a derivative thereof comprising at least 80% identity thereto.

28. The composition of claim 1, wherein at least one of said fragments comprise a mutation that increases stability or solubility of said fragment or a mutation that decreases aggregation of the fragment.

29. The composition of claim 28, wherein said mutation that increases stability comprises replacement of a cys loop within an acetylcholine receptor subunit with CDVSGVDTESGATNC (SEQ ID NO: 44) or wherein said acetylcholine receptor subunit is selected from: an alpha subunit comprising the amino acid sequence provided in SEO ID NO: 131, a beta subunit comprising the amino acid sequence provided in SEO ID NO: 132, a gamma subunit comprising the amino acid sequence provided in SEO ID NO: 133, a delta subunit comprising the amino acid sequence provided in SEO ID NO: 134, and an epsilon subunit comprising the amino acid sequence provided in SEO ID NO: 135.

30. (canceled)

31. (canceled)

32. The composition of claim 1, wherein said fragment comprises at least 20 sequential amino acids from said protein at least one B cell receptor (BCR)-specific epitope target of autoantibodies or both.

33. (canceled)

34. (canceled)

35. The composition of claim 28, wherein said fragment comprises mutation that decreases aggregation of the fragment and is selected from:
a. a fragment of ACHRA and comprises a mutation selected from: deletion of N141, F100G, W149R, V155A, Y93F, Y93H, Y93R and a combination thereof within a wild-type AChRa comprising SEQ ID NO: 1 or a AChRa with increased solubility comprising SEQ ID NO: 131;
b. a fragment of ACHRG and comprises a mutation selected from: M84S, Y105E, Y117E, Y117R, and a combination thereof within a wild-type AChRa comprising SEQ ID NO: 6 or a AChRa with increased solubility comprising SEQ ID NO: 133; and
c. a fragment of ACHRD and comprises a mutation selected from: C108A, C108I, Y119R, deletion of N141, L151E and a combination thereof within a wild-type AChRa comprising SEQ ID NO: 8 or a AChRa with increased solubility comprising SEQ ID NO: 134.

36. The composition of claim 1, wherein said first polypeptide chain and said second polypeptide chain are selected from: SEQ ID NO: 92 and SEQ ID NO: 93; SEQ ID NO: 95 and SEQ ID NO: 96; SEQ ID NO: 97 and SEQ ID NO: 98, SEQ ID NO: 99 and SEQ ID NO: 100, SEQ ID NO: 92 and SEQ ID NO: 102; SEQ ID NO 103 and SEQ ID NO: 100; SEQ ID NO: 105 and SEQ ID NO: 130; SEQ ID NO: 105 and SEQ ID NO: 106; and SEQ ID NO: 105 and SEQ ID NO: 107.

37. The composition of claim 22, wherein said first and second polypeptide chains are the same and are selected from: SEQ ID NO: 94, SEQ ID NO: 104, and SEQ ID NO: 108-129.

38. A polypeptide comprising a fragment of a first human acetylcholine receptor subunit comprising at least one mutation that decreases aggregation of the fragment, wherein said fragment is selected from:
a. a fragment of ACHRA and comprises a mutation selected from: deletion of N141, F100G, W149R, V155A, Y93F, Y93H, Y93R and a combination thereof within a wild-type AChRa comprising SEQ ID NO: 1 or a AChRa with increased solubility comprising SEQ ID NO: 131;
b. a fragment of ACHRG and comprises a mutation selected from: M84S, Y105E, Y117E, Y117R, and a combination thereof within a wild-type AChRa comprising SEQ ID NO: 6 or a AChRa with increased solubility comprising SEQ ID NO: 133; and
c. a fragment of ACHRD and comprises a mutation selected from: C108A, C108I, Y119R, deletion of N141, L151E and a combination thereof within a wild-type AChRa comprising SEQ ID NO: 8 or a AChRa with increased solubility comprising SEQ ID NO: 134.

39. The polypeptide of claim 38, wherein at least one of:
a. said polypeptide further comprises replacement of a cys loop within an acetylcholine receptor subunit with CDVSGVDTESGATNC (SEQ ID NO: 44) and wherein said subunit is ACHRA and said cys loop consists of CEIIVTHFPFDEQNC (SEQ ID NO: 39), said subunit is ACHRG and said cys loop consists of CSISVTYFPFDWQNC (SEQ ID NO: 41), or said subunit is ACHRD and said cys loop consists of CPISVTYFPFDWQNC (SEQ ID NO: 42),
b. said polypeptide further comprises a second fragment of a second acetylcholine receptor subunit linked to said first fragment by an amino acid linker; and optionally further comprising a fragment from a third, fourth, or fifth acetylcholine receptor subunit;
c. said polypeptide further comprises an Fc region of a human antibody heavy chain, optionally wherein said Fc region is separated from said fragment by an amino acid linker; and
d. said polypeptide comprises a sequence selected from SEO ID NO: 72-91.

40. (canceled)

41. (canceled)

42. (canceled)

43. A pharmaceutical composition comprising a composition of claim 1 and a pharmaceutically acceptable carrier, excipient or adjuvant; optionally wherein said pharmaceutical composition is formulated for systemic administration to a subject.

44. A method of treating myasthenia gravis in a subject in need thereof, the method comprising administering to said subject a pharmaceutical composition of claim 43, thereby treating myasthenia gravis.

45. The method of claim 44, wherein at least one of:
a. said method further comprise reducing in said subject the levels of circulating antibodies against at least said first human acetylcholine receptor subunit prior to said administering;
b. said method further comprises reducing in said subject the levels of circulating antibodies against a human acetylcholine receptor subunit within a protein complex comprising said first human acetylcholine receptor subunit or said second human acetylcholine receptor subunit;
c. said treating comprises decreasing the concentration of circulating autoantibodies against said human acetylcholine receptor subunits;
d. said treating comprises killing B cells producing said autoantibodies; and
e. said treating comprises killing autoreactive B cells producing autoantibodies against a fragment of said composition.

46. (canceled)

47. (canceled)

48. (canceled)

49. (canceled)

50. A nucleic acid system comprising a nucleic acid molecule, wherein a first nucleic acid molecule encodes said first polypeptide chain and a second nucleic acid molecule encodes said second polypeptide chain of a composition of claim 1.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. A method of determining suitability of a subject in need thereof to be treated, the method comprising:
receiving a sample from the subject,
contacting said sample with a composition comprising a protein complex comprising:
a. a first polypeptide chain comprising a fragment of a first human acetylcholine receptor subunit or an analog or derivative thereof and a first dimerization domain, and
b. a second polypeptide chain comprising a fragment of a second human acetylcholine receptor subunit or an analog or derivative thereof and a second dimerization domain,
and wherein said first and second subunits are different subunits, said analog or derivative thereof comprises at least 85% identity to said human protein and said first and second dimerization domains are configured to dimerize with each other; and
determining binding of autoantibodies against AChR within said sample to said composition,
wherein binding of autoantibodies against AChR to said composition indicates said subject is suitable to be treated by a method of claim 44, thereby determining suitability of the subject to be treated, optionally wherein binding of at least 20% of autoantibodies against AChR in the sample to said composition indicates said subject is suitable.

66. (canceled)
